(19) United States
(12) Patent Application Publication
(10) Pub. No.: US 2009/0275574 A1
(43) Pub. Date: Nov. 5, 2009

(54) NOVEL COMPOUNDS-300

(75) Inventors: Yun-Xing Cheng, Saint Laurent (CA); Vijayaratnam Santhakumar, Saint Laurent (CA); Miroslaw Jerzy Tomaszewski, Saint Laurent (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/432,818

(22) Filed: Apr. 30, 2009

Related U.S. Application Data
(60) Provisional application No. 61/050,343, filed on May 5, 2008.

(51) Int. Cl. A61K 31/517; C07D 401/04; A61K 31/538; A61P 25/28; A61P 25/18

(52) U.S. Cl. 514/230.5; 544/286; 544/105; 514/266.22

(57) ABSTRACT

Compounds of Formula I, or pharmaceutically acceptable salts thereof:

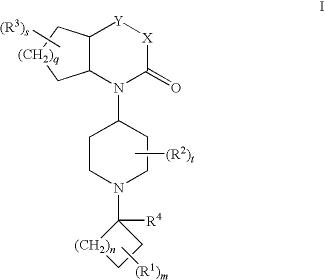

wherein R¹, R², R³, R⁴, m, n, q, s, t, X, and Y are as defined in the specification as well as salts and pharmaceutical compositions including the compounds are prepared. They are useful in therapy, in particular in the management of pain.

### BACKGROUND OF THE INVENTION

1. Field of the invention

The present invention relates to agonists of muscarinic receptors. The present invention also provides compositions comprising such agonists, and methods therewith for treating muscarinic receptor mediated diseases. Particularly, the present invention is related to compounds that may be effective in treating pain, Alzheimer's disease, and/or schizophrenia.

2. Discussion of Relevant Technology

The neurotransmitter acetylcholine binds to two types of cholinergic receptors: the ionotropic family of nicotinic receptors and the metabotropic family of muscarinic receptors. Muscarinic receptors belong to the large superfamily of plasma membrane-bound G protein coupled receptors (GPCRs) and show a remarkably high degree of homology across species and receptor subtype. These M1-M5 muscarinic receptors are predominantly expressed within the parasympathetic nervous system which exerts excitatory and inhibitory control over the central and peripheral tissues and participate in a number of physiologic functions, including heart rate, arousal, cognition, sensory processing, and motor control.

Muscarinic agonists such as muscarine and pilocarpine, and antagonists, such as atropine have been known for over a century, but little progress has been made in the discovery of receptor subtype-selective compounds, thereby making it difficult to assign specific functions to the individual receptors. See, e.g., DeLapp, N. et al., “Therapeutic Opportunities for Muscarinic Receptors in the Central Nervous System,” J. Med. Chem., 43(23), pp. 4333-4353 (2000); Hulme, E. C. et al., “Muscarinic Receptor Subtypes,” Ann. Rev. Pharmacol. Toxicol., 30, pp. 633-673 (1990); Caulfield, M. P. et al., “Muscarinic Receptors-Characterization, Coupling, and Function,” Pharmacol. Ther., 58, pp. 319-379 (1993); Caulfield, M. P. et al., International Union of Pharmacology. XVII. Classification of Muscarinic Acetylcholine Receptors,” Pharmacol. Rev., 50, pp. 279-290 (1998).

The Muscarinic family of receptors is the target of a large number of pharmacological agents used for various diseases, including leading drugs for COPD, asthma, urinary incontinence, glaucoma, schizophrenia, Alzheimer's (AchE inhibitors), and Pain.

For example, direct acting muscarinic receptor agonists have been shown to be antinociceptive in a variety of animal models of acute pain (Bartolini A., Ghelardini C., Fantetti L., Malcangio M., Malmberg-Aiello P., Giotti A. Role of muscarinic receptor subtypes in central antinociception. Br. J. Pharmacol. 105:77-82,1992; Capone F., Aloisi A. M., Carli G., Sacerdote P., Pavone F. Oxotremorine-induced modifications of the behavioral and neuroendocrine responses to formalin pain in male rats. Brain Res. 830:292-300,1999.).

A few studies have examined the role of muscarinic receptor activation in chronic or neuropathic pain states. In these studies, the direct and indirect elevation of cholinergic tone was shown to ameliorate tactile allodynia after intrathecal administration in a spinal ligation model of neuropathic pain in rats and these effects again were reversed by muscarinic antagonists (Hwang J.-H., Hwang K.-S., Leem J.-K., Park P.-H., Han S.-M., Lee D.-M. The antiallodynic effects of intrathecal cholinesterase inhibitors in a rat model of neuropathic pain. Anesthesiology 90:492-494, 1999; Lee E. J., Sim J. Y. Park J. Y., Hwang J. H., Park P. H., Han S. M. Intrathecal carbachol and clonidine produce a synergistic antiallodynic effect in rats with a nerve ligation injury. Can J Anaesth 49:178-84, 2002.). Thus, direct or indirect activation of muscarinic receptors has been shown to elicit both acute analgesic activity and to ameliorate neuropathic pain. Muscarinic agonists and ACHE-Is are not widely used clinically owing to their propensity to induced a plethora of adverse events when administered to humans. The undesirable side effects include excessive salivation and sweating, enhanced gastrointestinal motility, and bradycardia among other adverse events. These side effects are associated with the ubiquitous expression of the muscarinic family of receptors throughout the body.

### DESCRIPTION OF THE EMBODIMENTS

To date, five subtypes of muscarinic receptors (M1-M5) have been cloned and sequenced from a variety of species, with differential distributions in the body.

Therefore, it was desirable to provide molecules would permit selective modulation, for example, of muscarinic receptors controlling central nervous function without also activating muscarinic receptors controlling cardiac, gastrointestinal or glandular functions.

There is also a need for methods for treating muscarinic receptor-mediated diseases.

There is also a need for modulators of muscarinic receptors that are selective as to subtypes M1-M5.

The term “Cₘ₋ₙ” or “Cₘ₋ₙ group” refers to any group having m to n carbon atoms.

The term “alkyl” refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkylene” used alone or as a suffix or prefix, refers to divalent straight or branched chain hydrocarbon radicals comprising 1 to about 12 carbon atoms, which serves to links two structures together.

The term “alkenyl” refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “cycloalkyl” refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “aryl” refers to a monovalent hydrocarbon radical having one or more polyunsaturated carbon rings having aromatic character, (e.g., 4n+2 delocalized electrons) and comprising 5 up to about 14 carbon atoms.

The term “heterocycle” refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heterocyclyl” refers a monovalent radical derived from a heterocycle by removing one hydrogen therefrom.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “heteroaryl” refers to a heterocyclyl having aromatic character.

The term “heterocylcoalkyl” refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “six-membered” refers to a group having a ring that contains six ring atoms.

The term “five-membered” refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “alkoxy” refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.
“HATU” means O-(7-Azabenzotriazol-1-yl)-N,N,N′,N′-tetramethyluronium hexafluorophosphate.
“DCC” means N,N′-Dicyclohexylcarbodiimidide.
“EDC” means 1-Ethyl-3-(3-dimethylaminopropyl)carbodiimidehydrochloride.
“CDI” means 1,1′-Carbonyldiimidazole.
“DIPEA” means Diisopropylethylamine.

In certain embodiments, one or more compounds of the present invention may exist as two or more diastereomers (also called “diastereo isomer”) or enantiomers. These two or more diastereo isomers or enantiomers may be isolated using one or more methods described in the invention even though the absolute structures and configuration of these diastereo isomers or enantiomers may not be ascertained or determined. In order to identify and/or distinguish these diastereo isomers or enantiomers from each other, designations such as “diastereo isomer 1,” “diastereo isomer 2,” “diastereomer 1,” “diastereomer 2,” or “enantiomer 1,” “enantiomer 2” may be used to design the isolated isomers.

In one aspect, an embodiment of the invention provides a compound of Formula I, a pharmaceutically acceptable salt thereof, diastereomer, enantiomer, or mixture thereof:

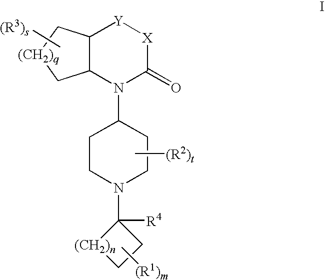

wherein

each R¹ is independently selected from fluoro, C₃₋₇cycloalkyl, C₁₋₇alkyl, C₂₋₆alkenyl, C₂₋₆alkynyl, C₁₋₇alkoxy, C₃₋₇cycloalkoxy-C₁₋₆alkyl, C₁₋₆alkoxy-C₁₋₆alkyl, C₂₋₆alkenyloxy, C₂₋₆alkenyloxy-C₁₋₆alkyl, C₂₋₆alkynyloxy, C₂₋₆alkynyloxy-C₁₋₆alkyl, C₁₋₆alkylamino, di-C₁₋₆alkylamino, C₃₋₇heterocycloalkyloxy, C₃₋₇heterocycloalkyl, C₆₋₁₀aryl-C₁₋₃alkoxy, C₆₋₁₀aryl-C₁₋₃alkyl, C₃₋₉heteroaryl-C₁₋₃alkoxy, C₃₋₉heteroaryl-C₁₋₃alkyl, C₃₋₇heterocycloalkyl-C₁₋₃alkoxy, C₃₋₇heterocycloalkyl-C₁₋₃alkyl, C₃₋₇cycloalkyloxy, C₃₋₇cycloalkyl-C₁₋₃alkyl, C₃₋₇cycloalkyl-C₁₋₃alkoxy and C₃₋₇cycloalkyl-C₁₋₃alkoxy-C₁₋₃alkyl, wherein said C₃₋₇cycloalkyl, C₁₋₇alkyl, C₂₋₆alkenyl, C₂₋₆alkynyl, C₁₋₇alkoxy, C₃₋₇cycloalkoxy-C₁₋₆alkyl, C₁₋₆alkoxy-C₁₋₆alkyl, C₂₋₆alkenyloxy, C₂₋₆alkenyloxy-C₁₋₆alkyl, C₂₋₆alkynyloxy, C₂₋₆alkynyloxy-C₁₋₆alkyl, C₁₋₆alkylamino, di-C₁₋₆alkylamino, C₃₋₇heterocycloalkyloxy, C₃₋₇heterocycloalkyl, C₆₋₁₀aryl-C₁₋₃alkoxy, C₆₋₁₀aryl-C₁₋₃alkyl, C₃₋₉heteroaryl-C₁₋₃alkoxy, C₃₋₉heteroaryl-C₁₋₃alkyl, C₃₋₇heterocycloalkyl-C₁₋₃alkoxy, C₃₋₇heterocycloalkyl-C₁₋₃alkyl, C₃₋₇cycloalkyloxy, C₃₋₇cycloalkyl-C₁₋₃alkyl, C₃₋₇cycloalkyl-C₁₋₃alkoxy and C₃₋₇cycloalkyl-C₁₋₃alkoxy-C₁₋₃alkyl are optionally substituted with one or more group selected from phenyl, C₃₋₆cycloalkyl, C₂₋₅heterocycloalkyl, C₃₋₅heteroaryl, —CN, —SR, —OR, —O(CH₂)ₚ—OR, R, —C(═O)—R, —CO₂R, —SO₂R, -SO₂NRR′, halogen, —NO₂, —NRR′, —(CH₂)ₚNRR′, and —C(═O)—NRR;

each R² is independently selected from halogen, C₁₋₆alkyl, C₃₋₇cycloalkyl, halogenated C₁₋₆alkyl, C₁₋₆alkoxy, and halogenated C₁₋₆alkoxy;

each R³ is independently selected from halogen, C₁₋₆alkyl, C₃₋₇cycloalkyl, halogenated C₁₋₆alkyl, CN, C₁₋₆alkoxy, and halogenated C₁₋₆alkoxy; or two R³ together form a C₁₋₆alkylene, C₁₋₆alkylenoxy, or halogenated C₁₋₆alkylene;

R⁴ is hydrogen, C₁₋₆ alkyl, or C₁₋₆haloalkyl;

q is 1, 2, 3 or 4;

p is 2, 3 or 4; s is 0, 1, 2, 3, or 4; t is 0, 1, 2, 3, or 4; n is 0,1, 2, 3 or 4; m is 0, 1, 2, 3 or 4;

Y is —CR⁵R⁶—, —O—, or —S—;

X is —CR⁵R⁵—, —NR⁷—, —O—, or —S—;

each R⁵, R⁶ and R⁷ are independently selected from hydrogen, C₁₋₆alkyl, C₂₋₆alkenyl and halogenated C₁₋₆alkyl; and

each R and R′ are independently C₁₋₆alkyl, C₂₋₆alkenyl or halogenated C₁₋₆alkyl, with a proviso that at least one of X and Y is —CR⁵R⁶—, with a further proviso that the compound is not (4aS,8aS)-4-(1-(4-(ethoxymethyl)-1-methylcyclohexyl)piperidin-4-yl)hexahydro-2H-benzo[b][1,4]oxazin-3(4H)-one.

In some embodiments:

Y is —CR⁵R⁵— or —O—; and

X is —CR⁵R⁶— or —NR⁷—.

In some embodiments, Y is —CR⁵R⁶—. In some embodiments, Y is —O—. In some embodiments, Y is —S—.

In some embodiments, X is —CR⁵R⁶—. In some embodiments, X is —NR⁷—. In some embodiments, X is —S—.

In some embodiments, X is not —O—

In some embodiments, X is —CH₂— or —NH—.

In some embodiments, Y is not —S—.

In some embodiments, when Y is —CR⁵R⁶— then X is not —CR⁵R⁶—; and when X is —CR⁵R⁶—, then Y is not —CR⁵R⁶—.

In some embodiments, when X is —CR⁵R⁶—, then Y is not —CR⁵R⁶—; and when Y is —CR⁵R⁶—, then X is not —CR⁵R⁶—.

In some embodiments, X is not —S—; Y is not —S—; when X is —CR⁵R⁶—, then Y is not —CR⁵R⁶—; and when Y is —CR⁵R⁶—, then X is not —CR⁵R⁶—.

In another embodiment, wherein R¹ is selected from C₁₋₆alkoxy, C₁₋₆alkoxy-C₁₋₆alkyl, halogenated C₁₋₆alkoxy-C₁₋₆alkyl, C₁₋₆alkyl, C₃₋₆alkenyloxy, C₃₋₆alkynyloxy, C₃₋₆cycloalkyl, C₃₋₆cycloalkyl-C₁₋₃alkoxy, halogenated C₁₋₆alkyl, halogenated C₃₋₆cycloalkyl-C₁₋₃alkoxy, or halogenated C₃₋₆cycloalkyl.

In another embodiment, R¹ is selected from ethyl, ethynyloxy, propyloxy, propoxymethyl, ethoxy, ethoxymethyl, isopropoxymethyl, cyclopropylmethoxy, and isopropyloxy.

In another embodiment, each R² is independently selected from methyl, fluoromethyl, difluoromethyl, trifluoromethyl, ethyl, C₁₋₃alkoxy and fluoro.

In another embodiment, each R³ is independently selected from methyl, fluoromethyl, difluoromethyl, trifluoromethyl, ethyl, C₁₋₃alkoxy and fluoro.

In some embodiments, R⁴ is hydrogen or C₁₋₆ alkyl.

In some embodiments, R⁴ is hydrogen, C₁₋₆alkyl, or fluorinated C₁₋₆haloalkyl.

In some embodiments, R⁴ is hydrogen or C₁₋₄alkyl.

In some embodiments, R⁴ is hydrogen, C₁₋₄alkyl, or fluorinated C₁₋₄haloalkyl

In some embodiments, R⁴ is hydrogen or C₁₋₃alkyl.

In some embodiments, R⁴ is hydrogen, C₁₋₃alkyl, or fluorinated C₁₋₃haloalkyl

In some embodiments, R⁴ is hydrogen or methyl.

In some embodiments, R⁴ is hydrogen, methyl, or fluorinated methyl.

In some embodiments, R⁴ is hydrogen, C₁₋₃alkyl, fluoromethyl, difluoromethyl, or trifluoromethyl.

In some embodiments, R⁴ is hydrogen, methyl, ethyl, fluoromethyl, difluoromethyl, or trifluoromethyl.

In some embodiments, R⁴ is hydrogen.

In a further embodiment, n is 1.

In another embodiment, n is 2.

In a further embodiment, n is 3.

In another embodiment, m is 1.

In another embodiment, t is 0.

In another embodiment, s is 0.

In another embodiment, q is 2.

In another embodiment, q is 1.

In a further embodiment, X is selected from NH and N—R, wherein R is C₂₋₃alkenyl, C₁₋₃alkyl, FCH₂CH₂—, F₂CHCH₂—, or CF₃CH₂—.

In another embodiment, Y is CH₂ or O.

In another embodiment, Y is O.

In another embodiment, Y is CH₂.

In another embodiment, X is O.

In another embodiment, X is NH.

In another embodiment, X is CH₂.

In a further embodiment, the invention provides a compound selected from
(4aR,8aS)-1-(1-(4-(propoxymethyl)cyclohexyl)piperidin-4-yl)octahydroquinazolin-2(1H)-one;
(4aR,8aS)-1-(1-(4-(isopropoxymethyl)cyclohexyl)piperidin-4-yl)octahydroquinazolin-2(1H)-one;
(4aR,8aS)-1-(1-(4-propoxycyclohexyl)piperidin-4-yl)octahydroquinazolin-2(1H)-one;
(4aR,8aS)-1-(1-(4-isopropoxycyclohexyl)piperidin-4-yl)octahydroquinazolin-2(1H)-one;
(4aR,8aS)-1-(1-(4-(ethoxymethyl)cyclohexyl)piperidin-4-yl)octahydroquinazolin-2(1H)-one;
(4aR,8aS)-1-(1-(4-(prop-2-ynyloxy)cyclohexyl)piperidin-4-yl)octahydroquinazolin-2(1H)-one;
(4aR,8aS)-1-(1-cyclopentylpiperidin-4-yl)octahydroquinazolin-2(1H)-one;
(4aR,8aS)-1-(1-(4-ethylcyclohexyl)piperidin-4-yl)octahydroquinazolin-2(1H)-one;
(4aR,8aS)-1-(1-cyclohexylpiperidin-4-yl)octahydroquinazolin-2(1H)-one;
(4aS,8aS)-4-(1-(4-(ethoxymethyl)cyclohexyl)piperidin-4-yl)hexahydro-2H-benzo[b][1,4]oxazin-3(4H)-one;
(4aS,8aS)-4-(1-(4-(isopropoxymethyl)cyclohexyl)piperidin-4-yl)hexahydro-2H-benzo[b][1,4]oxazin-3(4H)-one;
(4aS,8aS)-4-(1-(4-propoxycyclohexyl)piperidin-4-yl)hexahydro-2H-benzo[b][1,4]oxazin-3(4H)-one;
(4aS,8aS)-4-(1-(4-(isopropoxymethyl)cyclohexyl)piperidin-4-yl)hexahydro-2H-benzo[b][1,4]oxazin-3(4H)-one;
(4aS,8aS)-4-(1-(4-(cyclopropylmethoxy)cyclohexyl)piperidin-4-yl)hexahydro-2H-benzo[b][1,4]oxazin-3(4H)-one;
(4aS,8aS)-4-(1-(4-((cyclopropylmethoxy)methyl)cyclohexyl)piperidin-4-yl)hexahydro-2H-benzo[b][1,4]oxazin-3(4H)-one;
(4aS ,8aS)-4-(1-(4-((2-fluoroethoxy)methyl)cyclohexyl)piperidin-4-yl)hexahydro-2H-benzo[b][1,4]oxazin-3(4H)-one;
(4aS,8aS)-4-(1-(4-((2,2-difluoroethoxy)methyl)cyclohexyl)piperidin-4-yl)hexahydro-2H-benzo[b][1,4]oxazin-3(4H)-one;
(4aS,8aS)-4-(1-(4-((cyclobutylmethoxy)methyl)cyclohexyl)piperidin-4-yl)hexahydro-2H-benzo[b][1,4]oxazin-3(4H)-one;
(4aS,8aS)-4-(1-(4-(ethoxymethyl)-4-methylcyclohexyl)piperidin-4-yl)hexahydro-2H-benzo[b][1,4]oxazin-3(4H)-one;
(4aR,8aR)-4-(1-((1s,4S)-4-((cyclopropylmethoxy)methyl)cyclohexyl)piperidin-4-yl)hexahydro-2H-benzo[b][1,4]oxazin-3(4H)-one;
(cis)-4-(1-((1s,4S)-4-(ethoxymethyl)cyclohexyl)piperidin-4-yl)hexahydro-2H-benzo[b][1,4]oxazin-3(4H)-one;
(4aR,8aR)-6,6-difluoro-4-(1-(4-(isopropoxymethyl)cyclohexyl)piperidin-4-yl)hexahydro-2H-benzo[b][1,4]oxazin-3(4H)-one;
(4aS,8aS)-6,6-difluoro-4-(1-(4-(isopropoxymethyl)cyclohexyl)piperidin-4-yl)hexahydro-2H-benzo[b][1,4]oxazin-3(4H)-one;
(4aS,8aS)-4-(1-(3-(ethoxymethyl)cyclobutyl)piperidin-4-yl)hexahydro-2H-benzo[b][1,4]oxazin-3(4H)-one;
(4aS,8aS)-4-(1-(3-((cyclobutylmethoxy)methyl)cyclobutyl)piperidin-4-yl)hexahydro-2H-benzo[b][1,4]oxazin-3(4H)-one;
(4aS,8aS)-4-(1-(3-((cyclopropylmethoxy)methyl)cyclobutyl)piperidin-4-yl)hexahydro-2H-benzo[b][1,4]oxazin-3(4H)-one;
(4aS,8aS)-4-(1-((1R,3S)-3-(ethoxymethyl)cyclopentyl)piperidin-4-yl)hexahydro-2H-benzo[b][1,4]oxazin-3(4H)-one;
(4aS,8aS)-4-(1-((1S,3R)-3-(ethoxymethyl)cyclopentyl)piperidin-4-yl)hexahydro-2H-benzo[b][1,4]oxazin-3(4H)-one;
(4aS,8aS)-4-(1-(3-(ethoxymethyl)cyclopentyl)piperidin-4-yl)hexahydro-2H-benzo[b][1,4]oxazin-3(4H)-one;
(4aR,8aR)-4-(1-((1R,3S)-3-(ethoxymethyl)cyclopentyl)piperidin-4-yl)hexahydro-2H-benzo[b][1,4]oxazin-3(4H)-one;
(4aS,7aR)-4-(1-((1s,4R)-4-((cyclopropylmethoxy)methyl)cyclohexyl)piperidin-4-yl)hexahydrocyclopenta[b][1,4]oxazin-3(2H)-one;
4-(1-((1s,4R)-4-((cyclopropylmethoxy)methyl)cyclohexyl)piperidin-4-yl)hexahydrocyclopenta[b][1,4]oxazin-3(2H)-one;
(4aR,8aS)-1-(1-(4-((2,2-difluoroethoxy)methyl)cyclohexyl)piperidin-4-yl)octahydroquinazolin-2(1H)-one;
enantiomers thereof, diastereomers thereof, pharmaceutically acceptable salts thereof and mixtures thereof.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the Formula I.

It will also be understood that certain compounds of the present invention may exist in solvated, for example hydrated, as well as unsolvated forms. It will further be understood that the present invention encompasses all such solvated forms of the compounds of the Formula I.

Within the scope of the invention are also salts of the compounds of the Formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as agonists of M1 receptors. More particularly, the compounds of the invention exhibit selective activity as agonist of the M1 receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of M1 receptors is present or implicated. Furthermore, the compounds of the invention may be used to treat cancer, multiple sclerosis, Parkinson's disease, Huntington's chorea, schizophrenia, Alzheimer's disease, anxiety disorders, depression, obesity, gastrointestinal disorders and cardiovascular disorders.

In a particular embodiment, the compounds may be used to treat schizophrenia or Alzheimer's disease.

In another embodiment, the compounds may be used to treat pain.

In another particular embodiment, the compounds may be used to treat neuropathic pain.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of M1 receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastrointestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the Formula I above, for the manufacture of a medicament for the treatment of any of the conditions discussed above.

A further aspect of the invention is a method for the treatment of a subject suffering from any of the conditions discussed above, whereby an effective amount of a compound according to the Formula I above, is administered to a patient in need of such treatment.

Thus, the invention provides a compound of Formula I or pharmaceutically acceptable salt or solvate thereof, as hereinbefore defined for use in therapy.

In a further aspect, the present invention provides the use of a compound of Formula I or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain. In a particular embodiment, the compounds are useful in therapy for neuropathic pain. In an even more particular embodiment, the compounds are useful in therapy for chronic neuropathic pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (per cent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formula I as defined above for the manufacture of a medicament.

Also within the scope of the invention is the use of any compound of Formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I above, is administered to a patient in need of such therapy.

Additionally, there is provided a pharmaceutical composition comprising a compound of Formula I or a pharmaceutically acceptable salt thereof, in association with a pharmaceutically acceptable carrier.

Particularly, there is provided a pharmaceutical composition comprising a compound of Formula I or a pharmaceutically acceptable salt thereof, in association with a pharmaceutically acceptable carrier for therapy, more particularly for therapy of pain.

Further, there is provided a pharmaceutical composition comprising a compound of Formula I or a pharmaceutically acceptable salt thereof, in association with a pharmaceutically acceptable carrier use in any of the conditions discussed above.

In a further embodiment, a compound of the present invention, or a pharmaceutical composition or formulation comprising a compound of the present invention may be administered concurrently, simultaneously, sequentially or separately with one or more pharmaceutically active compound(s) selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(ii) atypical antipsychotics including for example quetiapine and pharmaceutically active isomer(s) and metabolite(s) thereof; amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, risperidone, quetiapine, sertindole, sulpiride, suproclone, suriclone, thioridazine, trifluoperazine, trimetozine, valproate, valproic acid, zopiclone, zotepine, ziprasidone and equivalents thereof;

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics including for example alnespirone, azapirones,benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including, for example, carbamazepine, valproate, lamotrogine, gabapentin and equivalents and pharmaceutically active isomer(s) and metabolite(s) thereof;

(vi) Alzheimer's therapies including, for example, donepezil, memantine, tacrine and equivalents and pharmaceutically active isomer(s) and metabolite(s) thereof;

(vii) Parkinson's therapies including, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies including, for example, abciximab, activase, NXY-059, citicoline, crobenetine, desmoteplase,repinotan, traxoprodil and equivalents and pharmaceutically active isomer(s) and metabolite(s) thereof;

(x) over active bladder urinary incontinence therapies including, for example, darafenacin, falvoxate, oxybutynin, propiverine, robalzotan, solifenacin, tolterodine and and equivalents and pharmaceutically active isomer(s) and metabolite(s) thereof;

(xi) neuropathic pain therapies including, for example, gabapentin, lidoderm, pregablin and equivalents and pharmaceutically active isomer(s) and metabolite(s) thereof;

(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos,secobarbital, zaleplon, zolpidem and equivalents and pharmaceutically active isomer(s) and metabolite(s) thereof; and

(xiv) mood stabilizers including, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combinations employ the compounds of this invention within the dosage range described herein and the other pharmaceutically active compound or compounds within approved dosage ranges and/or the dosage described in the publication reference.

In an even further embodiment, a compound of the present invention, or a pharmaceutical composition or formulation comprising a compound of the present invention may be administered concurrently, simultaneously, sequentially or separately with one or more pharmaceutically active compound(s) selected from buprenorphine; dezocine; diacetylmorphine; fentanyl; levomethadyl acetate; meptazinol; morphine; oxycodone; oxymorphone; remifentanil; sufentanil; and tramadol.

In a particular embodiment, it may be particularly effective to administrate a combination containing a compound of the invention and a second active compound selected from buprenorphine; dezocine; diacetylmorphine; fentanyl; levomethadyl acetate; meptazinol; morphine; oxycodone; oxymorphone; remifentanil; sufentanil; and tramadol to treat chronic nociceptive pain. The efficacy of this therapy may be demonstrated using a rat SNL heat hyperalgesia assay described below.

In a further aspect, the present invention provides a method of preparing the compounds of the present invention.

In one embodiment, the invention provides a process for preparing a compound of Formula I, comprising:

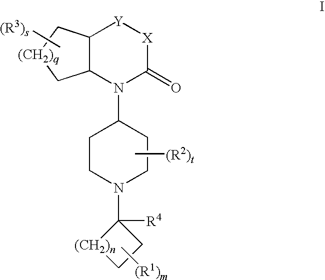

reacting a compound of Formula II with a compound of

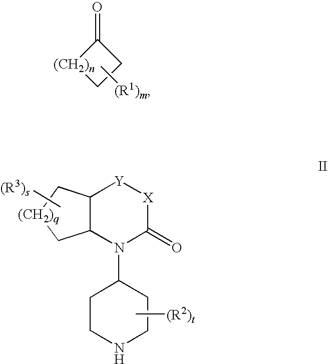

wherein R⁴ is hydrogen, and R¹, R², R³, m, n, q, s, t, Y and X are defined above.

Optionally, the step of reacting a compound of formula II with a compound of

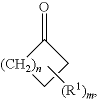

is carried out in the presence of a reducing agent, such as sodium triacetoxyborohydride, sodium borohydride, or equivalence thereof.

In another embodiment, certain compounds of the invention may be made according to the following scheme, wherein R¹, R², R³, R⁴, m, n, t, X and Y are as defined above.

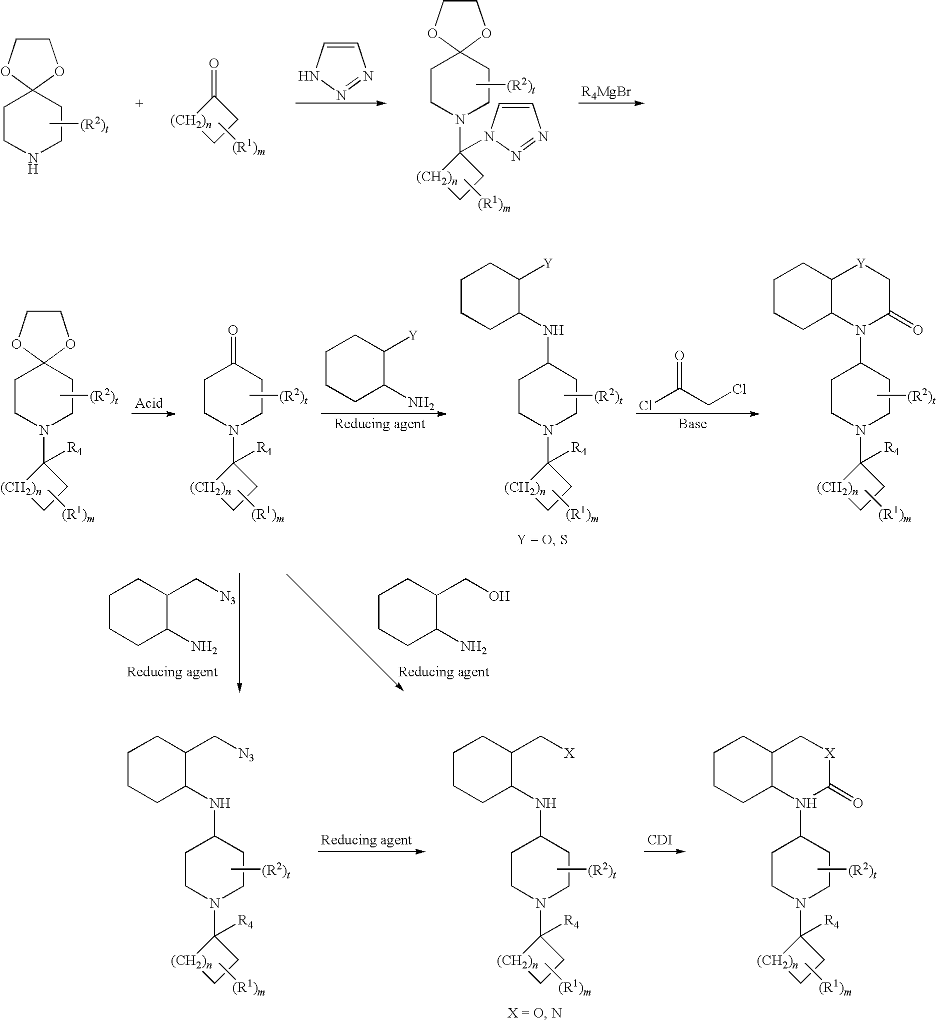

### Biological Evaluation

### Human M1, rat M1, Human M3 and Human M5 Calcium Mobilization FLIPR™ Assay

The compound activity in the present invention (EC50 or IC50) is measured using a 384 plate-based imaging assay that monitors drug induced intracellular Ca² release in whole cells. Activation of hM1 (human Muscarinic receptor subtype 1, gene bank access NM_{—}000738), rM1 (rat Muscarinic receptor subtype 1, gene bank access NM_{—}080773), hM3 (human Muscarinic receptor subtype 3, gene bank access NM_{—}000740NM_{—}000740) and hM5 (human Muscarinic receptor subtype 5, gene bank access NM_{—}0121258), receptors expressed in CHO cells (Chinese hamster ovary cells, ATCC) is quantified in a Molecular Devices FLIPR IITM instrument as an increase in fluorescent signal. Inhibition of hM3 and hM5 by compounds is determined by the decrease in fluorescent signal in response to 2 nM acetylcholine activation.

CHO cells are plated in 384-well black/clear bottom poly-D-lysine plates (Becton Dickinson, 4663) at 8000 cells/well/50 μl for 24 hours in a humidified incubator (5% CO2 and 37° C.) in DMEM/F12 medium (Wisent 319-075-CL) without selection agent. Prior to experiment, the cell culture medium is removed from the plates by inversion. A loading solution of 25 μl of Hank's balanced salt solution 1× (Wisent 311-506-CL), 10 mM Hepes (Wisent 330-050-EL) and 2.5 mM Probenicid at pH 7.4 (Sigma Aldrich Canada P8761-100 g) with 2 μM calcium indicator dye (FLUO-4AM, Molecular Probes F14202) and Pluronic acid F-127 0.002% (Invitrogen P3000MP) is added to each well. Plates are incubated at 37° C. for 60 minutes prior to start the experiment. The incubation is terminated by washing the cells four times in assay buffer, leaving a residual 25 μl buffer per well. Cell plates are then transferred to the FLIPR, ready for compound additions.

The day of experiment, acetylcholine and compounds are diluted in assay buffer in three-fold concentration range (10 points serial dilution) for addition by FLIPR instrument. For all calcium assays, a baseline reading is taken for 10 seconds followed by the addition of 12.5 μl of compounds, resulting in a total well volume of 37.5 μl. Data is collected every second for 60 pictures and then every 6 seconds for 20 pictures prior to the addition of agonist. For hM3 and hM5, before agonist addition, a second baseline reading is taken for 10 seconds followed by the addition of 12.5 μl of agonist or buffer, producing a final volume of 50 μl. After agonist stimulation, the FLIPR continues to collect data every second for 60 pictures and then every 6 seconds for 20 pictures. The fluorescence emission is read using filter 1 (emission 510-570 nm) by the FLIPR on board CCD camera.

Calcium mobilization output data are calculated as the maximal relative fluorescence unit (RFU) minus the minimal value for both compound and agonist reading frame (except for hM1 and rM1 using only the maximal RFU). Data are analyzed using sigmoidal fits of a non-linear curve-fitting program (XLfit version 4.2.2 Excel add-in version 4.2.2 build 18 math 1Q version 2.1.2 build 18). All pEC50 and pIC50 values are reported as arithmetic means±standard error of mean of ‘n’ independent experiments.

### hM2 Receptor GTPγS Binding

Membranes produced from Chinese hamster ovary cells (CHO) expressing the cloned human M2 receptor (human Muscarinic receptor subtype 2, gene bank access NM_{—}000739), are obtained from Perkin-Elmer (RBHM2M). The membranes are thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention are evaluated from 10-point dose-response curves (three fold concentration range) done in 60 μl in 384-well non-specific binding surface plate (Corning). Ten microliters from the dose-response curves plate (5× concentration) are transferred to another 384 well plate containing 25 μl of the following: 5 μg of hM2 membranes, 500 μg of Flashblue beads (Perkin-Elmer) and GDP 25 μM. An additional 15 μl containing 3.3× (60,000 dpm) of GTPγ³⁵S (0.4 nM final) are added to the wells resulting in a total well volume of 50 μl. Basal and maximal stimulated [³⁵S]GTPγS binding are determined in absence and presence of 30 μM final of acetylcholine agonist. The membranes/beads mix are pre-incubated for 15 minutes at room temperature with 25 μM GDP prior to distribution in plates (12.5 μM final). The reversal of acetylcholine-induced stimulation (2 μM final) of [³⁵S]GTPγS binding is used to assay the antagonist properties (IC₅₀) of the compounds. The plates are incubated for 60 minutes at room temperature then centrifuged at 400 rpm for 5 minutes. The radioactivity (cpm) is counted in a Trilux (Perkin-Elmer).

Values of EC₅₀, IC₅₀ and Eₘₐₓ are obtained using sigmoidal fits of a non-linear curve-fitting program (XLfit version 4.2.2 Excel add-in version 4.2.2 build 18 math 1Q version 2.1.2 build 18) of percent stimulated [³⁵S]GTPγS binding vs. log(molar ligand). All pEC50 and pIC50 values are reported as arithmetic means±standard error of mean of ‘n’ independent experiments.

### hM4 Receptor GTPγS Binding

Membranes produced from Chinese hamster ovary cells (CHO) expressing the cloned human M4 receptor (human Muscarinic receptor subtype 4, gene bank access NM_{—}000741), are obtained from Perkin-Elmer (RBHM4M). The membranes are thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention are evaluated from 10-point dose-response curves (three fold concentration range) done in 60 μl in 384-well non-specific binding surface plate (Corning). Ten microliters from the dose-response curves plate (5× concentration) are transferred to another 384 well plate containing 25 μl of the following: 10 μg of hM4 membranes, 500 μg of Flashblue beads (Perkin-Elmer) and GDP 40 μM. An additional 15 μl containing 3.3× (60,000 dpm) of GTPγ³⁵S (0.4 nM final) are added to the wells resulting in a total well volume of 50 μl. Basal and maximal stimulated [³⁵S]GTPγS binding are determined in absence and presence of 30 μM final of acetylcholine agonist. The membranes/beads mix are pre-incubated for 15 minutes at room temperature with 40 μM GDP prior to distribution in plates (20 μM final). The reversal of acetylcholine-induced stimulation (10 μM final) of [³⁵S]GTPγS binding is used to assay the antagonist properties (IC₅₀) of the compounds. The plates are incubated for 60 minutes at room temperature then centrifuged at 400 rpm for 5 minutes. The radioactivity (cpm) is counted in a Trilux (Perkin-Elmer).

Values of EC₅₀, IC₅₀ and Eₘₐₓ are obtained using sigmoidal fits of a non-linear curve-fitting program (XLfit version 4.2.2 Excel add-in version 4.2.2 build 18 math 1Q version 2.1.2 build 18) of percent stimulated [³⁵S]GTPγS binding vs. log(molar ligand). All pEC50 and pIC50 values are reported as arithmetic means±standard error of mean of ‘n’ independent experiments.

Certain biological properties of certain compounds of the invention measured using one or more assays described above are listed in Table 1 below.

TABLE 1
Certain Biological Properties of the Certain Compounds of the
Invention.

| | | | hM3 | hM4 | hM5 |
|---|---|---|---|---|---|
| Example | hM1 EC50 | hM2 EC50 | EC50 | EC50 | EC50 |
| Number | (nM) | (nM) | (nM) | (nM) | (nM) |
| Example 01 | 21 | 7700 | >40000 | >51940 | >40000 |
| Example 02 | 2 | 240 | 1600 | 520.9 | >2770 |
| Example 03 | 58 | 1700 | >40000 | | >14500 |
| Example 04 | 3.2 | 660 | 5100 | 683.5 | >40000 |
| Example 05 | 160 | | | | |
| Example 06 | 41 | 2500 | | >90000 | |
| Example 07 | 660 | | | | |
| Example 08 | 5.2 | 350 | 1100 | 985 | 69 |
| Example 09 | 31 | | | | |
| Example 10 | 68 | | | | |
| Example 11 | 43 | | >40000 | | |
| Example 12 | 60 | | >40000 | | |
| Example 13 | 17 | >21000 | >40000 | >23360 | >40000 |
| Example 14 | 9.9 | 2800 | >40000 | 4841 | >40000 |
| Example 15 | 10 | 3500 | >40000 | >33460 | >40000 |
| Example 16 | 190 | | | | |
| Example 17 | 88 | >48000 | | >90000 | |
| Example 18 | 7.8 | 3500 | >40000 | >24900 | >40000 |
| Example 19 | 14 | 8700 | >40000 | >43370 | >40000 |
| Example 20 | 170 | | | | |
| Example 21 | 110 | | | | |
| Example 22 | 5.6 | 1000 | | >90000 | |
| Example 23 | 38 | >1200 | | >90000 | |
| Example 24 | 41 | >5700 | >40000 | >90000 | >40000 |
| Example 25 | 9.6 | >2700 | >40000 | >26520 | >40000 |
| Example 26 | 17 | >3600 | >40000 | >90000 | >40000 |
| Example 27 | 4.2 | 870 | >40000 | >90000 | >40000 |
| Example 28 | 51 | >90000 | >40000 | | >40000 |
| Example 29 | 150 | >90000 | >40000 | | >40000 |
| Example 30 | 200 | | | | |
| Example 31 | 190 | | | | |
| Example 32 | 190 | | | | |
| Example 33 | 110 | | | | |
| Example 34 | 80 | | | | |
| Example 35 | 180 | | | | |
| Example 36 | 310 | | | | |
| Example 37 | 34 | >13000 | >40000 | >90000 | >40000 |
| Example 38 | 18 | >10000 | >40000 | >90000 | >40000 |
| Example 39 | 33 | 5000 | >40000 | >90000 | >40000 |
| Example 40 | 57 | 4400 | | >90000 | |
| Example 41 | 52 | >15000 | | | |
| Example 42 | 4.2 | 1600 | >2100 | 4237 | >4220 |
| Example 43 | 12 | >3900 | >40000 | >28150 | >40000 |
| Example 44 | 0.97 | 1600 | >10000 | 5355 | 2920 |
| Example 45 | 48 | 6100 | >40000 | >14720 | >40000 |
| Example 46 | 290 | | | | |
| Example 47 | 93 | | | | |
| Example 48 | 15 | 2200 | >40000 | >11740 | >40000 |
| Example 49 | 110 | | | | |
| Example 50 | 0.76 | 340 | | | |
| Example 51 | 23 | 3000 | >40000 | >90000 | >15700 |

### Rat SNL Heat Hyperalgesia Assay

Rats undergo spinal nerve ligation surgery as described in Kim and Chung (1992) (reference 1). Briefly, rats are anesthetized with isoflurane, the left L5 and L6 are isolated and tightly ligated with 4-0 silk thread. The wound is closed by suturing and applying tissue adhesive. Compound testing is performed at day 9 to day 36 post-surgery.

For behavioral testing, the animals are acclimatized to the test room environment for a minimum of 30 min. In order to assess the degree of hyperalgesia, the animals are placed on a glass surface (maintained at 30° C.), and a heat-source is focused onto the plantar surface of the left paw. The time from the initiation of the heat until the animal withdraws the paw is recorded. Each animal is tested twice (with an interval of 10 min between the two tests). A decrease in Paw Withdrawal Latency (PWL, average of the two tests) relative to naive animals indicates a hyperalgesic state. The rats with a PWL of at least 2 seconds less than average PWL of Naïve group are selected for compound testing.

Each individual experiment consists of several groups of SNL rats, one group receiving vehicle while the other groups receive different doses of the test article. In all experiments, animals are tested for heat hyperalgesia using the plantar test before drug or vehicle administration to ensure stable heat-hyperalgesia baseline and rats are evenly divided into groups for compound testing. At a suitable interval after vehicle or drug administration, another test is performed to measure PWL. Generally, results from 2 individual experiments are pooled together and the data are presented as the mean paw withdrawal latency (PWL) (s)±standard error of mean (SEM).

A combination containing a compound of the present invention and morphine at a predetermined ratio (e.g., 0.64:1) may be tested using this instant model. The combination drugs may be administered to the rats subcutaneously, orally or combination thereof, simultaneously or sequentially. The results (expressed as ED₅₀) for the combination may be compared with results obtained singly for the compound of the instant invention and morphine at the same or similar dosage range. If the ED₅₀ of the combination is significantly lower than the theoretical ED₅₀ calculated based on the ED₅₀ measured using the compound of the invention and morphine singly, then a synergy for the combination is indicated.

### EXAMPLES

The invention will further be described in more detail by the following Examples which describe methods whereby compounds of the present invention may be prepared, purified, analyzed and biologically tested, and which are not to be construed as limiting the invention.

Preparative LCMS Conditions: High pH LCMS purifications are run on Xbridge column with the following specification: XBridge Prep C18 OBD, 30×50, 5 um, run time: 10 min, mobile phases for high pH preparative LCMS are pH˜10 water and acetonitrile. pH˜10 water is prepared in the following fashion: dissolve 3.16 g NH₄HCO₃ (final concentraion of 10 mM), 15 mL concentrated ammonium hydroxide for every 4 L water. The gradient description in the experimental part, such as “High pH, 30-50% CH₃CN” means that the starting gradient for the run is 30% CH₃CN,/70% water for 1 minute, and then it goes to 50% CH₃CN/50% water in 7 minutes followed by a 2 minutes wash at 100% CH₃CN.

The compounds described in this application may be named with ChembridgeSoft naming program (Chemoffice 9.0.7) Chiral Super Critical Fluid Chromatography conditions: Chiral SFC are run on ChiralPak AD-H or ChiralPak AS-H with the following specifications: Dimensions of 10×250 mm, particle size 5 uM, Main eluent is CO₂ with mixture of co-eluents such as methanol, isopropanol and dimethylethylamine. Column temperature: 35° C., back pressure 100 Bar. Detection by UV at 215 nM wavelength.

### Intermediate Synthesis

### Intermediate 1: (4aR,8aS)-1-(piperidin-4-yl)octahydroquinazolin-2(1H)-one

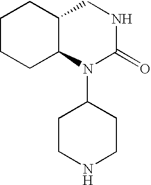

### Step A. Preparation of ((1S,2S)-2-(tert-butoxycarbonylamino)cyclohexyl)methyl methanesulfonate

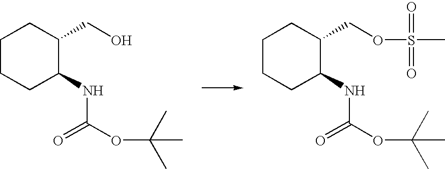

To a solution of tert-butyl (1S,2S)-2-(hydroxymethyl)cyclohexylcarbamate (10 g, 43.67 mmol) in dichloromethane (50 mL) was added methanesufonyl chloride (4 mL, 52 mmol) dropwise at 0° C. Triethylamine (7.35 mL, 52 mmol) was then added and the mixture was stirred at room temperature for 1 hour. The reaction was quenched with ice and diluted with dichloromethane. The organic phase was washed with saturated aqueous solution of NaHCO₃ and brine and dried. Concentrated in vacuo to provide the title compound as a brown solid (15 g), which was used in the subsequent step without further purification. MS (M+1): 308.16.

### Step B. Preparation of tert-butyl (1S,2R)-2-(azidomethyl)cyclohexylcarbamate

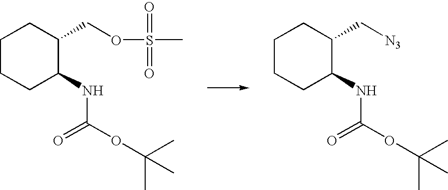

To a solution of ((1S,2S)-2-(tert-butoxycarbonylamino)cyclohexyl)methyl methanesulfonate (3 g, 9.76 mmol) in DMF (25 mL) was added sodium azide (1.27 g, 19.54 mmol). The mixture was heated at 120° C. for 3 hours. The reaction mixture was allowed to cool to room temperature and quenched with ice. The solvent was removed in vacuo. The residue was dissolved in ethyl acetate (100 mL) and washed with 1N NaOH (10 mL). The organic extract was dried and concentrated in vacuo to give the title compound (2.48 g), which was used in the subsequent step without further purification. MS (M+1): 255.21.

### Step C. Preparation of (1S,2R)-2-(azidomethyl)cyclohexanamine

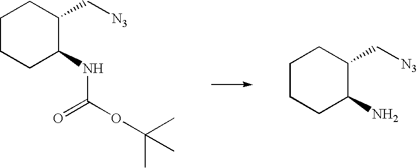

To a solution tert-butyl (1S,2R)-2-(azidomethyl)cyclohexylcarbamate (2.482 g, 9.76 mmol) in MeOH (20 mL) was added a solution of 4M HCl in dioxane (15 mL). The reaction mixture was stirred at room temperature over night. Concentrated in vacuo to give the title compound (2.2 g), which was used for the next step without further purification.

### Step D. Preparation of tert-butyl 4-((1S,2R)-2-(azidomethyl)cyclohexylamino)piperidine-1-carboxylate

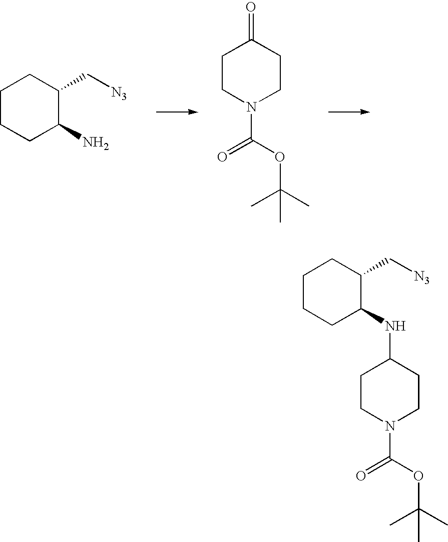

To a solution of (1S,2R)-2-(azidomethyl)cyclohexanamine (HCl salt, 7.53 mmol) in methanol (20 mL) was added tert-butyl 4-oxopiperidine-1-carboxylate (7.53 mmol) followed by sodium triacetoxy borohydride (3 g, 14.15 mmol). The reaction mixture was stirred at room temperature overnight. The reaction was quenched with 1N NaOH and diluted with dichloromethane. Phases were separated and aqueous phase was extracted several times with dichloromethane. The combined organic extract was dried and concentrated in vacuo to provide the title compound (2.48 g, 98%), which was used in the next step without further purification. MS (M+1): 338.3.

### Step E: Preparation of tert-butyl 4-((1S,2R)-2-(aminomethyl)cyclohexylamino)piperidine-1-carboxylate

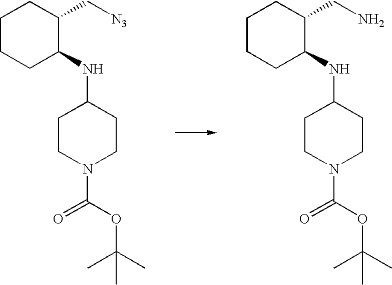

To a solution of tert-butyl 4-[4-[[(1S,2R)-2-(azidomethyl)cyclohexyl]amino]-1-piperidyl]piperidine-1-carboxylate (5.0 mmol) in MeOH (25 mL) was added Zn powder (6.5 g, 100 mmol) followed by NH₄Cl (1.36 g, 25 mmol). The reaction mixture was stirred at room temperature for 3 hours. The reaction mixture was filtered through Celite and the filtrate was concentrated in vacuo to give the title compound, which was used in the next step without further purification. MS (M+1): 312.3.

### Step F. Preparation of tert-butyl 4-((4aR,8aS)-2-oxooctahydroquinazolin-1(2H)-yl)piperidine-1-carboxylate

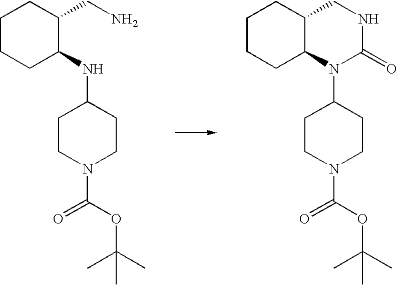

To a solution of tert-butyl 4-((1S,2R)-2-(aminomethyl)cyclohexylamino)piperidine-1-carboxylate (5 mmol) in MeCN (10 mL) was added 1,1′-carbonyldiimidazole (1.22 g, 7.5 mmol). The reaction mixture was stirred at room temperature for 12 hours. The solvent was removed in vacuo. Water (10 mL) was added to the residue followed by dichloromethane (80 mL). The phases were separated and the aqueous phase was extracted with dichloromethane (2×20 mL). The combined organic extract was washed with brine, dried over Na₂SO₄ and filtered (Standard aqueous work up). Concentrated in vacuo and the residue was purified by high pH preparative LC/MS to give the title compound as white solid (648 mg, 38% over two steps). MS (M+1): 338.2.

### Step G. Preparation of (4aR,8aS)-1-(piperidin-4-yl)octahydroquinazolin-2(1H)-one

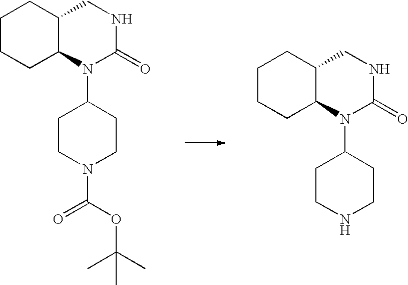

A solution of tert-butyl 4-((4aR,8aS)-2-oxooctahydroquinazolin-1(2H)-yl)piperidine-1-carboxylate (421 mg, 1.25 mmol) in 4N HCl in dioxane (5 mL) was stirred at room temperature for 3 hours. The reaction mixture was concentrated in vacuo to give the title compound (338 mg, 99%), which was used in the next step without further purification. MS (M+1): 238.2.

### Intermediate 2: (4aS,8aS)-4-(piperidin-4-yl)hexahydro-2H-benzo[b][1,4]oxazin-3(4H)-one

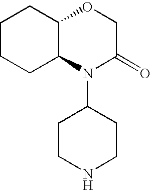

### Step A. Preparation of tert-butyl 4-((1S,2S)-2-(benzyloxy)cyclohexylamino)piperidine-1-carboxylate

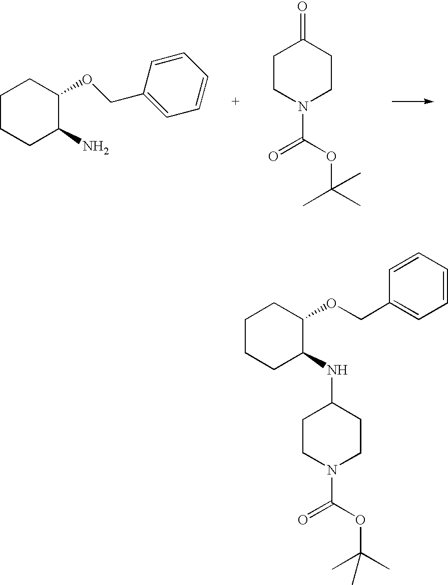

Following an analogous procedure to that described in Step D of Intermediate 1, the title compound was made from (1S,2S)-2-(benzyloxy)cyclohexanamine (3.75 g, 18.3 mmol) and tert-butyl 4-oxopiperidine-1-carboxylate (5.44 g, 18.3 mmol). The crude product (6.45 g, 91%) was used in the subsequent step without further purification. MS (M+1): 389.3.

### Step B. Preparation of tert-butyl 4-((1S,2S)-2-hydroxycyclohexylamino)piperidine-1-carboxylate

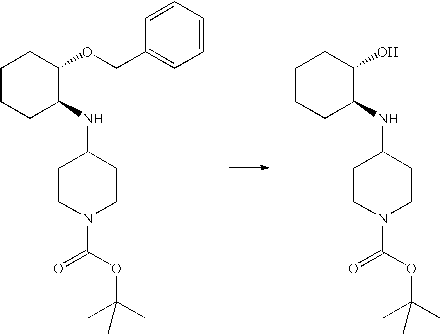

To a solution of tert-butyl 4-((1S,2S)-2-(benzyloxy)cyclohexylamino)piperidine-1-carboxylate (16.6 mmol) in EtOH (80 mL) was added cyclohexene (20 mL) followed by 20% Pd(OH)₂/C (0.5 g). The reaction mixture was heated under reflux for 12 hours. Solid materials were filtered off and the filtrate was concentrated in vacuo to give the title compound as white solid (5.24 g, 98%), which was used for the next step without further purification. MS (M+1): 299.1.

### Step C. Preparation of tert-butyl 4-(2-chloro-N-((1S,2S)-2-hydroxycyclohexyl)acetamido)piperidine-1-carboxylate

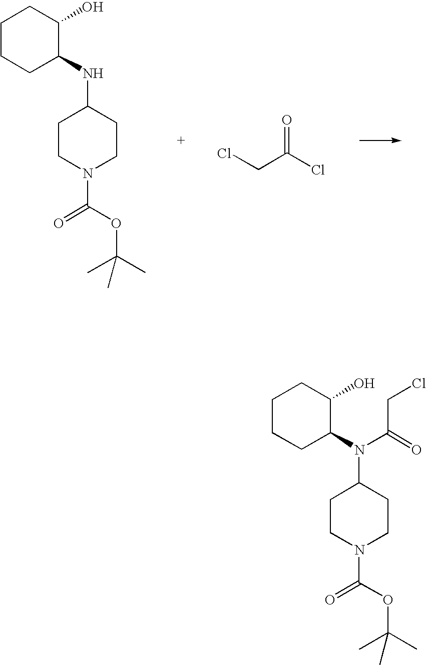

To a solution of tert-butyl 4-((1S,2S)-2-hydroxycyclohexylamino)piperidine-1-carboxylate (895 mg, 3.0 mmol) in dichloromethane (30 mL) was added 2-chloroacetyl chloride (0.32 mL, 4.1 mmol) followed by triethyl amine (0.46 mL, 3.3 mmol). The reaction mixture was stirred at room temperature for 18 hours. After the standard aqueous work up, the title compound was used in the subsequent step without further purification (1.08 g, 96%). MS (M+1): 375.2.

### Step D. Preparation tert-butyl 4-((4aS,8aS)-3-oxo-2H-benzo[b][1,4]oxazin-4(3H,4aH,5H,6H,7H,8H,8aH)-yl)piperidine-1-carboxylate

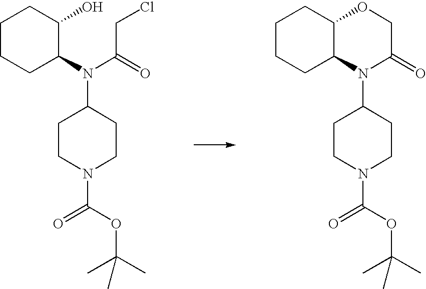

To a solution of tert-butyl 4-(2-chloro-N-((1S,2S)-2-hydroxycyclohexyl)acetamido)piperidine-1-carboxylate (1.08g, 2.88 mmol) in dry THF (30 mL) at 0° C. was added ^{t}BuOK (5.76 mmol). The reaction mixture was allowed to warm to room temperature and stirred at room temperature for 12 hours. After the standard work up, the crude product was used in the subsequent step without further purification (0.81 g, 83%). MS (M+1): 339.3.

### Step E. Preparation of (4aS,8aS)-4-(piperidin-4-yl)hexahydro-2H-benzo[b][1,4]oxazin-3(4H)-one

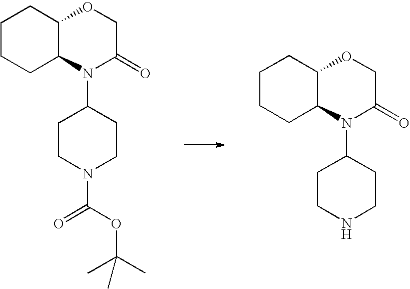

A mixture of tert-butyl 4-((4aS,8aS)-3-oxo-2H-benzo[b][1,4]oxazin-4(3H,4aH,5H,6H,7H,8H,8aH)-yl)piperidine-carboxylate (0.4 mmol) in 4N HCl (2 mL) was stirred at room temperature for 5 hours. Concentrated in vacuo to give the title compound, which was used in the next step without further purification. MS (M+1): 239.2.

### Intermediate 3: 4-(propoxymethyl)cyclohexanone

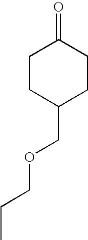

### Step A: Preparation of ethyl 1,4-dioxaspiro[4.5]decane-8-carboxylate

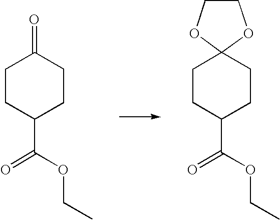

A mixture of ethyl 4-oxocyclohexanecarboxylate (5.4755 g, 32.17 mmol), ethylene glycol (4.13 mL, 73.99 mmol), and concentrated sulfuric acid (0.1 mL, 1.88 mmol) in toluene (55 mL) was heated under reflux for 16 hours with removal of water by a Dean Stark trap. After the standard work up, the title compound was obtained as a pale yellow oil (5.51 g, 80%), which was used in the subsequent step without further purification.

### Step B: Preparation of 1,4-dioxaspiro[4.5]decan-8-ylmethanol

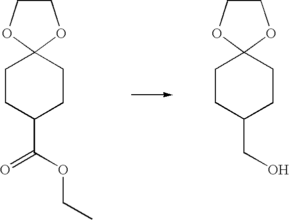

A solution of ethyl 1,4-dioxaspiro[4.5]decane-8-carboxylate (5.5053 g, 25.69 mmol) in diethyl ether (50 mL) was cooled in an ice bath. Lithium aluminum hydride (1.336 g, 35.20 mmol) was added to the solution in portions over 15 minutes. The mixture was warmed to room temperature and stirred at room temperature for 27 hours. Water (1.3 mL), 15% NaOH (1.3 mL) and water (3.9 mL) were added successively to the reaction mixture slowly. Na₂SO₄ was added to the mixture, and the reaction was filtered through a pad of Celite. The solids were washed well with Et₂O, and the filtrate was concentrated in vacuo give the title compound (4.15 g, 94%) as a colorless liquid. 1H NMR (400 MHz, CHLOROFORM-D) δ ppm 1.14-1.32 (m, 2 H), 1.45-1.59 (m, 3 H), 1.67 (s,1 H), 1.71-1.81 (m, 4 H), 3.47 (d, J=6.6 Hz, 2H), 3.86-3.98 (m, 4 H).

### Step C: Preparation of 8-(propoxymethyl)-1,4-dioxaspiro[4.5]decane

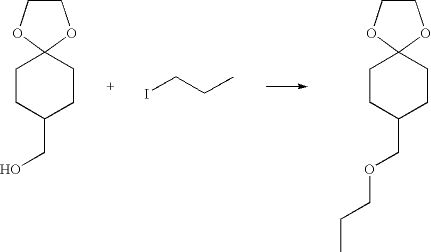

A mixture of 1,4-dioxaspiro[4.5]decan-8-ylmethanol (0.4879 g, 2.83 mmol), 1-iodopropane (1.105 mL, 11.33 mmol), and crushed potassium hydroxide (0.636 g, 11.33 mmol) in DMSO (5 mL) was stirred at room temperature for 69 hours. Brine (15 mL) and diethyl ether (20 mL) were added to the reaction mixture. The layers separated, and the aqueous layer was extracted with additional diethyl ether (2×20 mL). The combined organic extract was washed with brine (15 mL), dried over Na₂SO₄, and concentrated in vacuo to give the title compound (0.584 g, 96%) as a light yellow oil, which was used in the subsequent step without further purification.

### Step D: Preparation of 4-(propoxymethyl)cyclohexanone

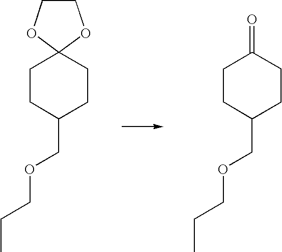

To a solution of 8-(propoxymethyl)-1,4-dioxaspiro[4.5]decane (0.5842 g, 2.73 mmol) in THF (12 mL) was added 3 M HCl (2.5 mL, 7.50 mmol). The reaction mixture was stirred at room temperature for 19 hours. The reaction mixture was concentrated in vacuo. Diethyl ether (10 mL) was added to the residue and the mixture was loaded onto a solid phase extraction cartridge. The cartridge was eluted with diethyl ether (3×8 mL). The eluant was concentrated in vacuo. The residue was purified by silica gel column chromatography (3-30% EtOAc:heptane) to give the title compound (0.229 g, 49.3%) as a colorless oil. 1H NMR (400 MHz, CHLOROFORM-D) δ ppm 0.93 (t, J=7.4 Hz, 3 H), 1.37-1.53 (m, 2 H), 1.53-1.71 (m, 2 H), 1.94-2.20 (m, 3 H), 2.25-2.50 (m, 4 H), 3.33 (d, J=6.2 Hz, 2 H), 3.40 (t, J=6.6 Hz, 2 H).

### Intermediate 4: 4-(ethoxymethyl)cyclohexanone

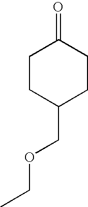

### Step A: Preparation of 8-(ethoxymethyl)-1,4-dioxaspiro[4.5]decane

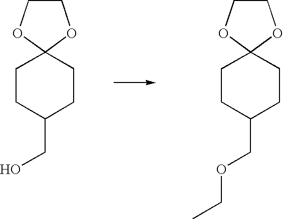

Following an analogous procedure to that described in Step C of Intermediate 3, the title compound was made from 1,4-dioxaspiro[4.5]decan-8-ylmethanol (0.3131 g, 1.82 mmol) and iodoethane (0.582 mL, 7.27 mmol). The title compound (0.340 g, 93%) was obtained as a light yellow oil, which was used in the subsequent step without further purification. 1H NMR (400 MHz, CHLOROFORM-D) δ ppm 1.11-1.30 (m, 5 H), 1.43-1.67 (m, 3 H), 1.68-1.84 (m, 4 H), 3.23 (d, J=6.6 Hz, 2 H), 3.44 (q, J=6.8 Hz, 2 H), 3.84-3.99 (m, 4 H).

### Step B: Preparation of 4-(ethoxymethyl)cyclohexanone

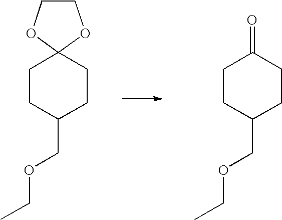

Following an analogous procedure to that described in Step D of Intermediate 3, the title compound was made from 8-(ethoxymethyl)-1,4-dioxaspiro[4.5]decane (0.364 g, 1.82 mmol). The title compound (0.211 g, 74.5%) was obtained as a colorless oil. 1H NMR (400 MHz, CHLOROFORM-D) δ ppm 1.19 (t, J=7.0 Hz, 3 H), 1.34-1.50 (m, 2 H), 1.94-2.06 (m, 1H), 2.06-2.16 (m, 2 H), 2.25-2.43 (m, 4 H), 3.31 (d, J=6.6 Hz, 2 H), 3.47 (q, J=7.0 Hz, 2 H).

### Intermediate 5: 4-(isopropoxymethyl)cyclohexanone

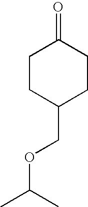

### Step A: Preparation of 8-(isopropoxymethyl)-1,4-dioxaspiro[4.5]decane

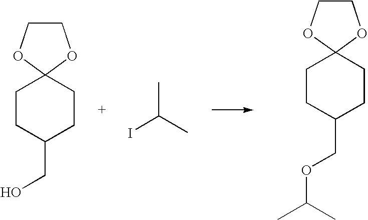

A mixture of 1,4-dioxaspiro[4.5]decan-8-ylmethanol (0.4374 g, 2.54 mmol), 2-iodopropane (1.977 ml, 19.81 mmol), and silver(I) oxide (1.104 g, 4.76 mmol) was stirred at room temperature with protection from light for 141 hours. Et₂O (5 mL) was added to the reaction mixture and filtered. The solid was washed well with Et₂O, and the filtrate was concentrated in vacuo. The residue was partitioned between water (20 mL) and hexanes (20 mL). The layers were separated, the organic layer was dried over Na₂SO₄, filtered, and concentrated in vacuo to give the title compound (0.476 g, 87%) as a colorless liquid, which was used in the subsequent step without further purification.

### Step B: Preparation of 4-(isopropoxymethyl)cyclohexanone

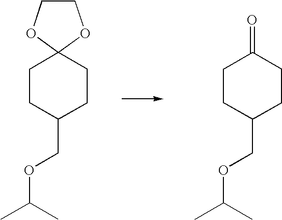

Following an analogous procedure to that described in Step D of Intermediate 3, the title compound was made from 8-(isopropoxymethyl)-1,4-dioxaspiro[4.5]decane (0.4757 g, 2.22 mmol). The crude product was purified by flash chromatography (3-30% EtOAc:heptane) to give the title compound (0.251 g, 66.5%) as a colorless liquid. 1H NMR (400 MHz, CHLOROFORM-D) ppm 1.16 (d, J=6.2 Hz, 6 H), 1.35-1.51 (m, 2 H), 1.92-2.07 (m, 1 H), 2.08-2.19 (m, 2 H), 2.28-2.45 (m, 4 H), 3.32 (d, J=6.6 Hz, 2 H), 3.48-3.61 (m, 1H).

### Intermediate 6: 4-propoxycyclohexanone

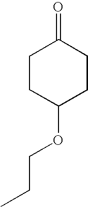

### Step A: Preparation of 4-dioxaspiro[4.5]decan-8-ol

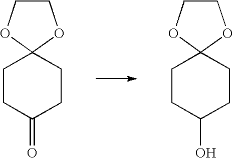

A solution of 4-dioxaspiro[4.5]decan-8-on (5.0134 g, 32.10 mmol) in methanol (100 mL) was cooled in an ice bath. Sodium borohydride (3.64 g, 96.30 mmol) was added in portions over 20 minutes to the solution. The mixture was stirred at 0° C. for 30 minutes. The reaction mixture was warmed to room temperature and stirred at room temperature for 1 hour. After the standard work up, the title compound (5.56 g, 109%) was obtained as yellow oil, which was used in the subsequent step without further purification. 1H NMR (400 MHz, CHLOROFORM-D) δ ppm 1.47-1.70 (m, 5 H), 1.72-1.92 (m, 4 H), 3.70-3.83 (m,1H), 3.85-3.96 (m, 4 H).

### Step B: Preparation of 8-propoxy-1,4-dioxaspiro[4.5]decane

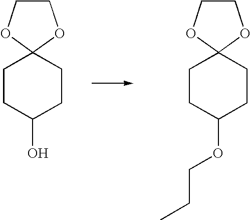

Following an analogous procedure to that described in Step C of Intermediate 3, the title compound was made from 1,4-dioxaspiro[4.5]decan-8-ol (0.3865 g, 2.44 mmol) and 1-iodopropane (0.953 mL, 9.77 mmol). The title compound (0.346 g, 70.6%) was obtained as a pale yellow oil, which was used in the subsequent step without further purification. 1H NMR (400 MHz, CHLOROFORM-D) δ ppm 0.90 (t, J=7.4 Hz, 3 H), 1.46-1.62 (m, 4 H), 1.62-1.75 (m, 2 H), 1.74-1.85 (m, 4 H), 3.32-3.40 (m, 2 H), 3.86-3.97 (m, 4 H).

### Step C: Preparation of 4-propoxycyclohexanone

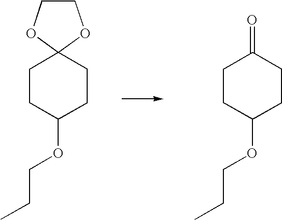

Following an analogous procedure to that described in Step D of Intermediate 3, the title compound was made from 8-propoxy-1,4-dioxaspiro[4.5]decane (0.3456 g, 1.73 mmol). The crude product was purified by flash chromatography (3-30% EtOAc:heptane) to give the title compound (0.162 g, 60.0%) as a colorless oil. 1H NMR (400 MHz, CHLOROFORM-D) δ ppm 0.94 (t, J=7.4 Hz, 3 H), 1.54-1.68 (m, 2 H), 1.85-1.97 (m, 2 H), 1.99-2.13 (m, 2 H), 2.18-2.30 (m, 2 H), 2.50-2.63 (m, 2 H), 3.44 (t, J=6.6 Hz, 2 H), 3.64-3.72 (m, 1 H).

### Intermediate 7: 4-isopropoxycyclohexanone

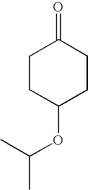

### Step A: Preparation of 8-isopropoxy-1,4-dioxaspiro[4.5]decane

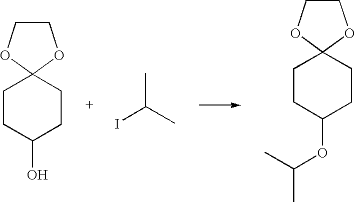

Following an analogous procedure to that described in Step A of Intermediate 5, the title compound was made from 1,4-dioxaspiro[4.5]decan-8-ol (0.4401 g, 2.78 mmol) and 2-iodopropane (2.166 ml, 21.70 mmol). The title compound (0.449 g, 81%) was obtained as a pale yellow liquid, which was used in the subsequent step without further purification. 1H NMR (400 MHz, CHLOROFORM-D) δ ppm 1.12 (d, J=6.2 Hz, 6 H), 1.45-1.57 (m, 2 H), 1.57-1.72 (m, 2 H), 1.72-1.85 (m, 4 H), 3.38-3.50 (m, 1 H), 3.58-3.70 (m, 1H), 3.86-3.97 (m, 4 H).

### Step B: Preparation of 4-isopropoxycyclohexanone

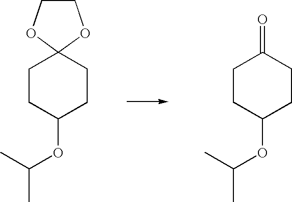

Following an analogous procedure to that described in Step D of Intermediate 3, the title compound was made from 8-isopropoxy-1,4-dioxaspiro[4.5]decane (0.4489 g, 2.24 mmol). The crude product was purified by flash column chromatography (3-30% EtOAc:heptane) to give the title compound (0.278 g, 80%) as a colorless oil. 1H NMR (400 MHz, CHLOROFORM-D) δ ppm 1.17 (d, J=6.2 Hz, 6 H), 1.81-2.05 (m, 4 H), 2.16-2.31 (m, 2 H), 2.50-2.65 (m, 2 H), 3.67-3.75 (m,1H), 3.75-3.81 (m, 1 H).

### Intermediate 8: 4-(prop-2-ynyloxy)cyclohexanone

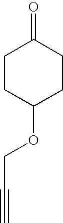

### Step A: Preparation of 8-(prop-2-ynyloxy)-1,4-dioxaspiro[4.5]decane

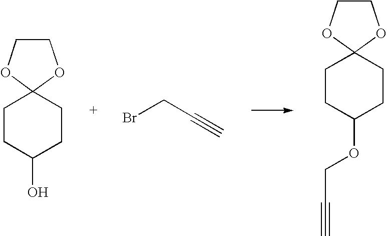

Following an analogous procedure to that described in Step C of Intermediate 3, the title compound was made from 1,4-dioxaspiro[4.5]decan-8-ol (0.4080 g, 2.58 mmol) and 3-bromoprop-1-yne (80% weight in xylene) (0.286 mL, 2.58 mmol). The crude product was purified by flash chromatography (3-30% EtOAc:Heptane) to give the title compound (0.085 g, 16.75%) as a colorless oil. 1H NMR (400 MHz, CHLOROFORM-D) δ ppm 1.46-1.62 (m, 2 H), 1.64-1.92 (m, 6 H), 2.38 (t, J=2.3 Hz, 1 H), 3.56-3.70 (m,1 H), 3.83-3.99 (m, 4 H), 4.15 (d, J=2.3 Hz, 2 H).

### Step B: Preparation of 4-(prop-2-ynyloxy)cyclohexanone

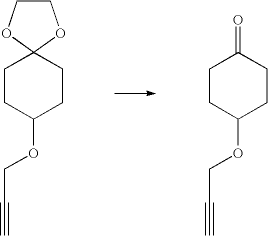

Following an analogous procedure to that described in Step D of Intermediate 3, the title compound was made from 8-(prop-2-ynyloxy)-1,4-dioxaspiro[4.5]decane (0.3009 g, 1.53 mmol). The title compound (0.214 g, 92%) was obtained as white solid. 1H NMR (400 MHz, CHLOROFORM-D) δ ppm 1.88-2.01 (m, 2 H), 2.04-2.17 (m, 2 H), 2.21-2.36 (m, 2 H), 2.43 (t, J=2.5 Hz, 1H), 2.50-2.69 (m, 2 H), 3.88-4.02 (m, 1 H), 4.24 (d, J=2.3 Hz, 2 H).

### Intermediate 9: 4-(cyclopropylmethoxy)cyclohexa none

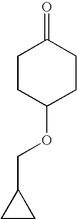

### Step A: Preparation of 8-(cyclopropylmethoxy)-1,4-dioxaspiro[4.5]decane

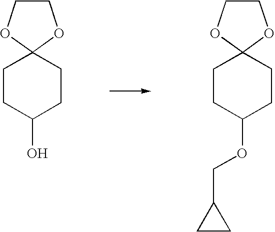

Following an analogous procedure to that described in Step C of Intermediate 3, the title compound was made from 1,4-dioxaspiro[4.5]decan-8-ol (0.411 g, 2.60 mmol) and (bromomethyl)cyclopropane (0.3 mL, 3.09 mmol). The crude product (0.544 g, 99%) was used in the subsequent step without further purification.

### Step B: Preparation of 4-(cyclopropylmethoxy)cyclohexanone

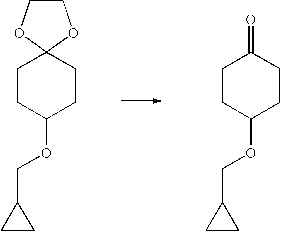

Following an analogous procedure to that described in Step D of Intermediate 3, the title compound was made from 8-(cyclopropylmethoxy)-1,4-dioxaspiro[4.5]decane (0.5440 g, 2.56 mmol). The crude product was purified by silica gel column chromatography (3-30% EtOAc:heptane) to give the title compound (0.196 g, 45.5%). 1H NMR (400 MHz, CHLOROFORM-D) δ ppm 0.14-0.33 (m, 2 H), 0.47-0.64 (m, 2 H), 0.98-1.18 (m, 1 H), 1.88-2.01 (m, 2 H), 2.02-2.16 (m, 2 H), 2.27 (dt, J=14.6, 6.2 Hz, 2 H), 2.59 (ddd, J=14.8, 9.8, 5.9 Hz, 2 H), 3.35 (d, J=7.0 Hz, 2 H), 3.74 (tt, J=5.9, 2.9 Hz, 1 H).

### Intermediate 10: 4-((cyclopropylmethoxy)methyl)cyclohexanone

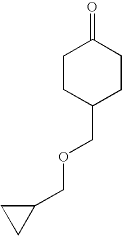

### Step A: Preparation of 8-((cyclopropylmethoxy)methyl)-1,4-dioxaspiro[4.5]decane

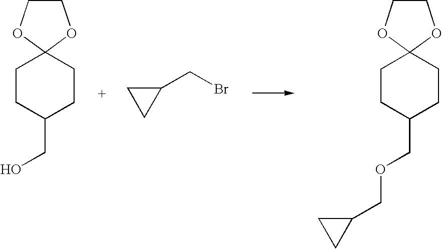

Following an analogous procedure to that described in Step C of the Intermediate 3 the title compound (0.599 g, 102%) was made from 1,4-dioxaspiro[4.5]decan-8-ylmethanol (0.447 g, 2.60 mmol), and (bromomethyl)cyclopropane (0.3 mL, 3.09 mmol). The crude product was used in the subsequent step without further purification.

### Step B: Preparation of 4-((cyclopropylmethoxy)methyl)cyclohexanone

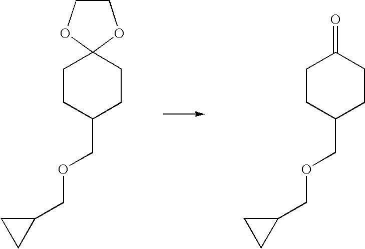

Following an analogous procedure to that described in Step D of Intermediate 3, the title compound was made from 8-((cyclopropylmethoxy)methyl)-1,4-dioxaspiro[4.5]decane (0.599 g, 2.65 mmol). The crude product was purified by silica gel column chromatography to give the title compound (0.148 g, 30.6%). 1H NMR (400 MHz, CHLOROFORM-D) δ ppm 0.16-0.25 (m, 2 H), 0.46-0.60 (m, 2 H), 1.36-1.52 (m, 2 H), 1.97-2.26 (m, 4 H), 2.28-2.46 (m, 4 H), 3.28 (d, J=7.0 Hz, 3.35 (d, J=6.2 Hz, 2 H).

### Intermediate 11: 4-((2-fluoroethoxy)methyl)cyclohexanone

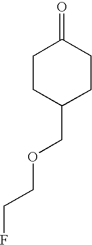

### Step A: Preparation of 8-((2-fluoroethoxy)methyl)-1,4-dioxaspiro[4.5]decane

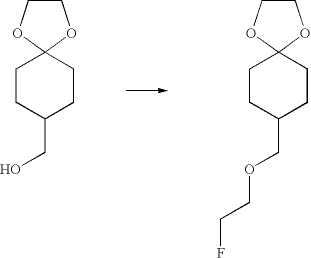

Sodium hydride (60% in mineral oil) (0.087 g, 2.18 mmol) was washed with pentane and then suspended in dry DMSO (2 mL) under a nitrogen atmosphere. A solution of 1,4-dioxaspiro[4.5]decan-8-ylmethanol (0.3407 g, 1.98 mmol) in dry DMSO (3 mL) was added, and the resulting mixture was stirred for 10 minutes at room temperature. 2-Fluoroethyl 4-methylbenzenesulfonate (0.432 g, 1.98 mmol) was then added, and the reaction mixture was stirred at 75° C. for 2 hours. Water (5 mL) was cautiously added, followed by Et₂O (50 mL). The layers were separated, and the organic layer was washed with brine (3×10 mL). The organic layer was dried over Na₂SO₄, filtered, and concentrated under reduced pressure. The residue was purified by silica gel column chromatography (EtOAc/heptane mixture) to provide the title compound (0.137 g, 31.7%). 1H NMR (400 MHz, CHLOROFORM-D) δ ppm 1.18-1.36 (m, 2 H), 1.47-1.87 (m, 7 H), 3.35 (d, J=6.6 Hz, 2 H), 3.59-3.77 (m, 2 H), 3.88-400 (m, 4 H), 4.46-4.65 (m, 2 H).

### Step B: Preparation of 4-((2-fluoroethoxy)methyl)cyclohexanone

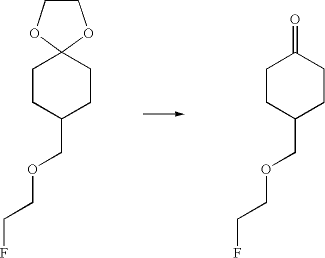

Following an analogous procedure to that described in Step D of Intermediate 3, the title compound was made from 8-((2-fluoroethoxy)methyl)-1,4-dioxaspiro[4.5]decane (0.1246 g, 0.57 mmol). The crude product was purified by silica gel column chromatography (5-60% EtOAc:heptane) to provide the title compound (0.074 g, 73.9%). 1H NMR (400 MHz, CHLOROFORM-D) δ ppm 1.38-1.54 (m, 2 H), 1.98-2.22 (m, 3 H), 2.28-2.51 (m, 4 H), 3.43 (d, J=6.2 Hz, 2 H), 3.62-3.81 (m, 2 H), 4.44-4.69 (m, 2 H).

### Intermediate 12: 4-((2,2-difluoroethoxy)methyl)cyclohexanone

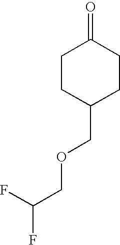

### Step A: Preparation of N3-(1-(4-((2,2-difluoroethoxy)methyl)cyclohexyl)piperidin-4-yl)pyridine-3,4-diamine

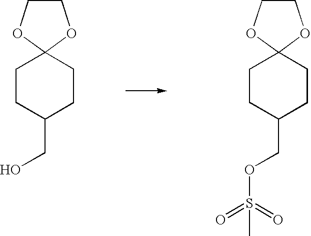

A solution of 1,4-dioxaspiro[4.5]decan-8-ylmethanol (0.767 g, 4.46 mmol) and triethylamine (0.7 mL, 4.90 mmol) in dichloromethane (23 mL) was cooled in an ice bath under a nitrogen atmosphere. Methanesulfonyl chloride (0.35 mL, 4.68 mmol) was added slowly to the solution and the mixture was stirred at 0° C. for 1 hour and at room temperature for 4 hours. The mixture was diluted with dichloromethane (100 mL) and was then washed successively with 1N NaOH (20 mL) and brine (20 mL). The layers were separated and organic phase was dried over Na₂SO₄, filtered, and concentrated under reduced pressure to give the title compound (1.126 g, 101%), which was used in the subsequent reaction without further purification. 1H NMR (400 MHz, CHLOROFORM-D) δ ppm 1.22-1.47 (m, 2 H), 1.56 (td, J=13.4, 4.5 Hz, 2 H), 1.71-1.89 (m, 5 H), 3.01 (s, 3 H), 3.88-4.01 (m, 4 H), 4.07 (d, J=6.2 Hz, 2 H).

### Step B: Preparation of 8-((2,2-difluoroethoxy)methyl)-1,4-d ioxaspiro[4.5]decane

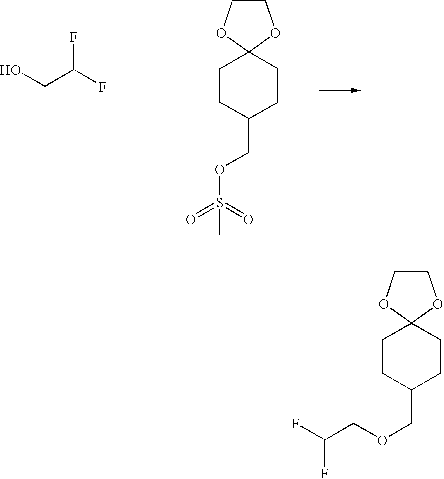

Sodium hydride (60% in mineral oil) (0.180 g, 4.50 mmol) was washed with pentane and then suspended in dry THF (2 mL) under a nitrogen atmosphere. A solution of 2,2-difluoroethanol (0.369 g, 4.50 mmol) in dry THF (4 mL) was added, and the resulting mixture was stirred for 30 minutes at room temperature. A solution of 1,4-dioxaspiro[4.5]decan-8-ylmethyl methanesulfonate (0.5627 g, 2.25 mmol) in dry THF (4 mL) was then added, and the reaction was heated at reflux for 50 hours. The reaction was cooled to room temperature, and a saturated solution of NH₄Cl (10 mL) was added slowly. The mixture was concentrated under reduced pressure to remove THF. Ethyl acetate (15 mL) was added to the aqueous residue, and the mixture was loaded onto a hydromatrix solid phase extraction cartridge. The product was eluted with ethyl acetate (3×12 mL) and the filtrate was concentrated under reduced pressure. The residue was purified by silica gel column chromatography (0-100% EtOAc:heptane) to give the title product (0.314 g, 59.1%) as a slightly yellow liquid. 1H NMR (400 MHz, CHLOROFORM-D) δ ppm 1.19-1.36 (m, 2 H), 1.46-1.88 (m, 7 H), 3.38 (d, J=6.6 Hz, 2 H), 3.64 (td, J=14.0, 4.1Hz, 2 H), 3.86-4.02 (m, 4 H), (tt, J=55.5, 4.2 Hz, 1H).

### Step C: Preparation of 4-((2,2-difluoroethoxy)methyl)cyclohexanone

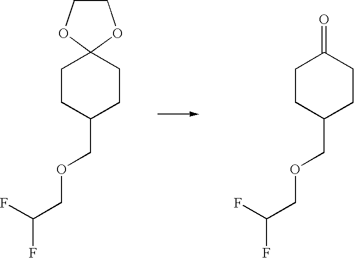

Following an analogous procedure to that described in Step D of Intermediate 3, the title compound was made from 8-((2,2-difluoroethoxy)methyl)-1,4-dioxaspiro[4.5]decane (0.313 g, 1.33 mmol). The crude product was purified by silica gel column chromatography (5-60% EtOAc:heptane) to provide the title compound (0.235 g, 92%). 1H NMR (400 MHz, CHLOROFORM-D) δ ppm 1.37-1.55 (m, 2 H), 1.98-2.17 (m, 3 H), 2.29-2.50 (m, 4 H), 3.47 (d, J=6.2 Hz, 2 H), 3.67 (td, J=14.0, 4.1 Hz, 2 H), 5.87 (tt, J=55.4, 4.0 Hz, 1 H).

### Intermediate 13: 4-((cyclobutylmethoxy)methyl)cyclohexanone

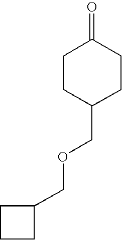

### Step A: Preparation of 8-((cyclobutylmethoxy)methyl)-1,4-dioxaspiro[4.5]decane

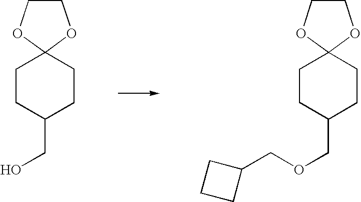

Following an analogous procedure to that described in Step C of the Intermediate 3 the title compound was made from 1,4-dioxaspiro[4.5]decan-8-ylmethanol (0.5168 g, 3.00 mmol) and (bromomethyl)cyclobutane (0.405 mL, 3.60 mmol). The crude product (0.475 g, 65.9%) was used in the subsequent step without further purification.

### Step B: Preparation of 4-((cyclobutylmethoxy)methyl)cyclohexanone

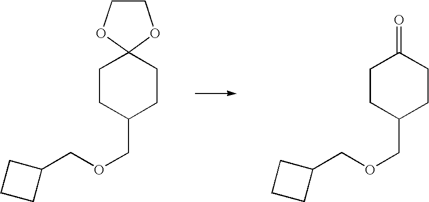

Following an analogous procedure to that described in Step D of Intermediate 3, the title compound was made from 8-((cyclobutylmethoxy)methyl)-1,4-dioxaspiro[4.5]decane (0.4752 g, 1.98 mmol). The crude product was purified by flash chromatography on silica gel, eluting with mixtures of EtOAc and heptane to afford the title compound (0.106 g, 27.4%). 1H NMR (400 MHz, CHLOROFORM-D) δ ppm 1.36-1.51 (m, 2 H), 1.66-2.18 (m, 9 H), 2.28-2.46 (m, 4 H), 2.51-2.64 (m, 1 H), 3.33 (d, J=6.6 Hz, 2 H), 3.41 (d, J=6.6 Hz, 2 H).

### Intermediate 14: 4-(ethoxymethyl)-4-methylcyclohexanone

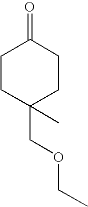

### Step A: Preparation of ethyl 8-methyl-1,4-dioxaspiro[4.5]decane-8-carboxylate

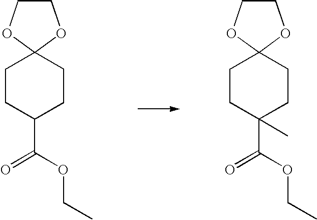

A solution of lithium diisopropylamide (1.666 mL, 3.33 mmol) in THF (10 mL) was cooled with a −78° C. bath. A solution of ethyl 1,4-dioxaspiro[4.5]decane-8-carboxylate (0.3569 g, 1.67 mmol) in THF (10 mL) was added slowly and the mixture was stirred for 30 minutes. lodomethane (0.26 mL, 4.16 mmol) was added, and the mixture was stirred for an additional 2 hours at −78° C. Water (10 mL) was added, and the reaction was warmed to room temperature. Et₂O was added (15 mL), the layers were separated, and the aqueous layer was extracted with additional Et₂O (2×15 mL). The combined organic layers were dried over Na₂SO₄, filtered, and concentrated under reduced pressure. The crude product was purified by flash column chromatography (5-50% EtOAc:heptane) to give the title compound (0.327 g, 86%). 1H NMR (400 MHz, CHLOROFORM-D) δ ppm 1.19 (s, 3 H), 1.23-1.29 (m, 3 H), 1.44-1.74 (m, 6 H), 2.09-2.19 (m, 2 H), 3.94 (s, 4 H), 4.15 (q, J=7.3 H, 2 H).

### Step B: Preparation of (8-methyl-1,4-dioxaspiro[4.5]decan-8-yl)methanol

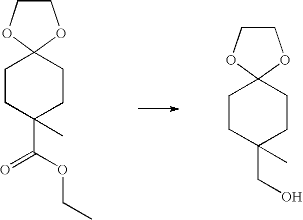

Following an analogous procedure to that described in Step B of Intermediate 3, the title compound was made from ethyl 8-methyl-1,4-dioxaspiro[4.5]decane-8-carboxylate (0.327 g, 1.43 mmol). The crude product (0.264 g, 99%) was used in the subsequent step without further purification. 1HNMR (400 MHz, CHLOROFORM-D) δ ppm 0.97 (s, 3 H), 1.35-1.46 (m, 2 H), 1.48-1.76 (m, 7 H), 3.41 (d, J=6.2 Hz, 2 H), 3.77-4.05 (m, 4 H).

### Step C: Preparation of 8-(ethoxymethyl)-8-methyl-1,4-dioxaspiro[4.5]decane

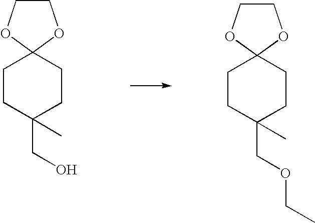

Following an analogous procedure to that described in Step C of Intermediate 3, the title compound was made from (8-methyl-1,4-dioxaspiro[4.5]decan-8-yl)methanol (0.254 g, 1.36 mmol). The crude product (0.304 g, 104%) was used in the subsequent step without further purification. 1H NMR (400 MHz, CHLOROFORM-D) δ ppm 0.97 (s, 3 H), 1.18 (t, J=7.0 Hz, 3 H), 1.34-1.46 (m, 2 H), 1.50-1.73 (m, 6 H), 3.17 (s, 2 H), 3.47 (q, J=7.0 Hz, 2 H), 3.94 (s, 4 H).

### Step D: Preparation of 4-(ethoxymethyl)-4-methylcyclohexanone

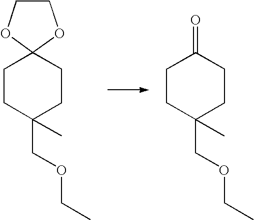

Following an analogous procedure to that described in Step D of Intermediate 3, the title compound was made from 8-(ethoxymethyl)-8-methyl-1,4-dioxaspiro[4.5]decane (0.3 g, 1.39 mmol). The crude product was purified by flash chromatography on silica gel, (EtOAc/heptane ) to afford the title compound (0.175 g, 74.2%). 1 H NMR (400 MHz, CHLOROFORM-D) δ ppm 1.11 (s, 3 H), 1.20 (t, J=7.0 Hz, 3 H), 1.61-1.73 (m, 2 H), 1.83 (ddd, J=14.3, 8.8, 6.2 Hz, 2 H), 2.24-2.50 (m, 4 H), 3.26 (s, 2 H), 3.49 (q, J=7.0 Hz, 2 H).

### Intermediate 15: 3-(ethoxymethyl)cyclopentanone

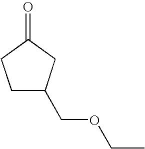

### Step A: Preparation of ethyl 1,4-dioxaspiro[4.4]nonane-7-carboxylate

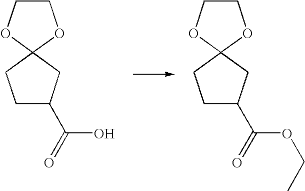

A mixture of 3-oxocyclopentanecarboxylic acid (0.6402 g, 5.00 mmol), ethylene glycol (0.557 mL, 9.99 mmol), triethyl orthoformate (0.416 mL, 2.50 mmol), and p-toluenesulfonic acid monohydrate (0.048 g, 0.25 mmol) in toluene (7 mL) was heated at reflux for 24 hours with removal of water by a Dean Stark trap. The reaction mixture was concentrated under reduced pressure, and the residue was partitioned between diethyl ether (30 mL) and a saturated solution of NaHCO₃ (10 mL). The organic layer was washed with water (10 mL), dried over Na₂SO₄, and concentrated under reduced pressure. The crude product was used in the subsequent step without further purification.

### Step B: Preparation of 1,4-dioxaspiro[4.4]nonan-7-ylmethanol

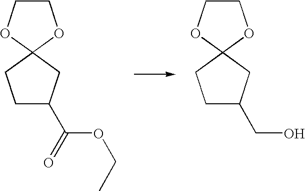

Following an analogous procedure to that described in Step B of Intermediate 3, the title compound was made from ethyl 1,4-dioxaspiro[4.4]nonane-7-carboxylate (0.8631 g, 4.31 mmol). The crude product was purified by silica gel column chromatography (25-100% EtOAc/heptane) to provide the title compound (0.293 g, 43.0%). 1H NMR (400 MHz, CHLOROFORM-D) δ ppm 1.39-1.54 (m, 1H), 1.55-1.67 (m, 2 H), 1.74-1.95 (m, 3 H), 1.96-2.09 (m, 1H), 2.19-2.41 (m, 1 H), 3.50-3.67 (m, 2 H), 3.83-4.01 (m, 4 H).

### Step C: Preparation of 7-(ethoxymethyl)-1,4-dioxaspiro[4.4]nonane

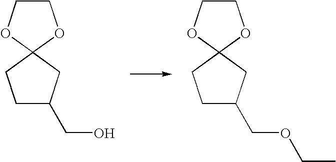

Following an analogous procedure to that described in Step C of Intermediate 3, the title compound was made from 1,4-dioxaspiro[4.4]nonan-7-ylmethanol (0.2855 g, 1.80 mmol). The crude product (0.361 g, 107%) was used in the subsequent step without further purification. 1H NMR (400 MHz, CHLOROFORM-D) δ ppm 1.19 (t, J=7.0 Hz, 3 H), 1.36-1.49 (m,1H), 1.50-1.60 (m,1H), 1.72-1.94 (m, 3 H), 2.00 (dd, J=13.7, 8.6 Hz, 1 H), 2.23-2.40 (m, 1 H), 3.29-3.39 (m, 2 H), 3.44-3.52 (m, 2 H), 3.84-3.96 (m, 4 H).

### Step D: Preparation of 3-(ethoxymethyl)cyclopentanone

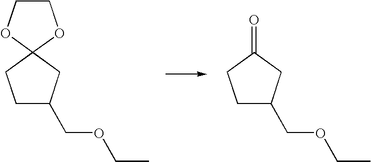

Following an analogous procedure to that described in Step D of Intermediate 3, the title compound was made from 7-(ethoxymethyl)-1,4-dioxaspiro[4.4]nonane (0.335 g, 1.8 mmol). The crude product was purified by silica gel column chromatography (3-30% EtOAc:heptane) to provide the title compound (0.202 g, 79%). 1H NMR (400 MHz, CHLOROFORM-D) δ ppm 1.20 (t, 3 H), 1.67-1.81 (m, 1H), 2.02 (ddd, J=18.4, 8.6, 1.6 Hz, 1H), 2.08-2.24 (m, 2 H), 2.25-2.42 (m, 2 H), 2.44-2.59 (m,1H), 3.41-3.46 (m, 2 H), 3.49 (q, J=7.0 Hz, 2 H).

### Intermediate 16: ((1s,4s)-4-(4,4-diethoxypiperidin-1-yl)cyclohexyl)methanol

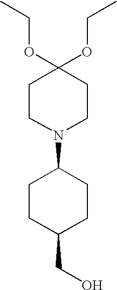

### Step A: Preparation of (1s,4s)-ethyl 4-(4-oxopiperidin-1-yl)cyclohexanecarboxylate

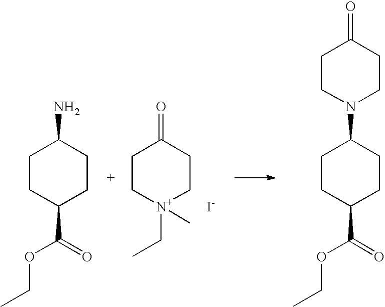

A mixture of (1s,4s)-ethyl 4-aminocyclohexanecarboxylate (12.07 g, 70.5 mmol) and potassium carbonate (9.72 g, 70.05 mmol) in ethanol (150 mL) was stirred at reflux for 15 minutes. A solution of 1-ethyl-1-methyl-4-oxopiperidinium iodide in water (75 mL) was added, the resulting mixture was stirred at reflux for 3 hours. The mixture was concentrated under reduced pressure. Dichloromethane (100 mL) and aqueous solution of NaHCO₃ (5%, 100 mL) were added to the reaction mixture and the phases were separated. The aqueous phase was extracted with dichloromethane (3×100 mL). The combined organic extracts were washed with brine, dried over sodium sulphate, filtered and concentrated under reduced pressure. The residue was purified by flash chromatography on silica gel, (0-10% of methanol (containing 1% NH₄OH) in dichloromethane) to afford the title compound (11.78 g, 66%). 1H NMR (300 MHz, CHLOROFORM-D) δ ppm 1.26 (t, J =7.12 Hz, 3 H), 1.44-1.72 (m, 6 H), 2.13-2.21 (m, 2 H), 2.40-2.59 (m, 2 H), 2.42 (t, J =5.99 Hz, 4 H), 2.82 (t, J =6.00 Hz, 4 H), 4.14 (q, J =7.12 Hz, 2 H).

### Step B: Preparation of (1s,4s)-ethyl 4-(4,4-diethoxypiperidin-1-yl)cyclohexanecarboxylate

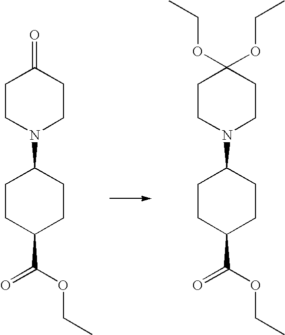

A mixture of (1s,4s)-ethyl 4-(4-oxopiperidin-1-yl)cyclohexanecarboxylate (11.3 g, 44.6 mmol) in dichloromethane (100 mL) was stirred at 0° C. Triethyl orthoformate(37.09 mL, 22.30 mmol) was added to the mixture followed by p-toluene sulfonic acid at 0° C. and the resulting mixture was stirred at room temperature overnight. The reaction mixture was added to NaHCO₃ (5%, 150 mL), and the phases were separated. The aqueous phase was extracted with dichloromethane (3×100 mL). The combined organic extracts were washed with brine, dried over sodium sulfate, filtered and concentrated under reduced pressure to afford the title product (11.4 g, 78%). 1H NMR (300 MHz, CHLOROFORM-D) δ ppm 1.16 (t, J =7.06 Hz, 6 H), 1.25 (t, J=7.11 Hz, 3 H), 1.43-1.80 (m, 11 H), 2.14-2.55 (m, 3H), 2.46-2.55 (m, 4 H), 3.45 (q, J=7.07 Hz, 4 H), 4.13 (q, J =7.13 Hz, 2 H).

### Step C: Preparation of ((1s,4s)-4-(4,4-diethoxypiperidin-1-yl)cyclohexyl)methanol

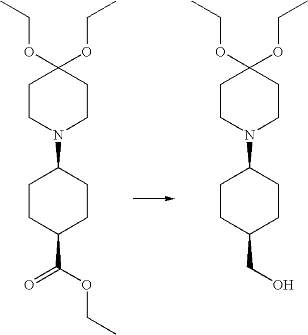

A mixture of lithium aluminum hydride (2.84 g, 74.8 mmol) in tetrahydrofuran was stirred at 0° C. under a nitrogen atmosphere. A solution of (1s,4s)-ethyl 4-(4,4-diethoxypiperidin-1-yl)cyclohexanecarboxylate (14.4 g, 44.0 mmol) in tetrahydrofuran (25 mL) was added, and the resulting mixture was stirred at room temperature overnight. Water (2.8 mL), a solution of sodium hydroxide (15%, 8.4 mL) and water (8.4 mL) were added successively at 0° C. to the reaction mixture, and the reaction mixture was stirred for 15 minutes. Magnesium sulphate (25 g) was then added to the reaction mixture, and stirred for 30 minutes. The reaction mixture was filtered and concentrated under reduced pressure to afford the title product (10.6 g, 85%). 1H NMR (300 MHz, CHLOROFORM-D) δ ppm 1.15 (t, J =7.05 Hz, 6 H), 1.38-1.78 (m, 12 H), 1.96-2.00 (m,1 H), 2.19-2.26 (m,1 H), 2.45-2.52 (m, 4 H), 3.43 (t, J=7.05 Hz, 4 H), 3.50-3.55 (m, 2 H). MS m/z 286.47 [M+H]+ (ESI).

### Intermediate 17: (4aR,8aR)-6,6-difluoro-4-(piperidin-4-yl)hexahydro-2H-benzo[b][1,4]oxazin-3(4H)-one

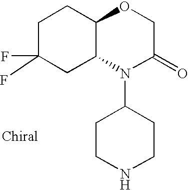

### Step A: Preparation of trans-7-(benzylamino)-1,4-dioxaspiro[4.5]decan-8-ol

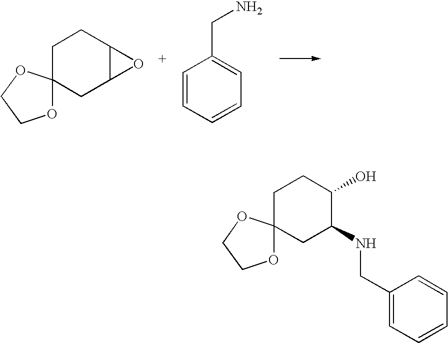

A mixture of 7-oxaspiro[bicyclo[4.1.0]heptane-3,2′-[1,3]dioxolane] (4.81 g, 30.80 mmol) (prepared by known method: C. Y. Cheng, S. C. Wu, L. W. Hsin, S. W. Tam; Journal of Medicinal Chemistry (1992), 35(12), 2243-7) and phenylmethanamine (3.92 g, 36.58 mmol) in iPrOH (60 mL) under a nitrogen atmosphere was stirred at reflux for 24 hours. The reaction mixture was concentrated under reduced pressure. The residue was purified by flash chromatography on silica gel (dichloromethane and MeOH) to afford the title product (4.85 g, 60%). 1H NMR (400 MHz, CHLOROFORM-D) δ ppm 1.24-1.40 (m, 1 H), 1.41-1.84 (m, 5 H), 1.84-2.04 (m, 1 H), 2.15 (dt, J=12.79, 3.56 Hz, 1 H), 2.60 (ddd, J=11.91, 9.57, 4.30 Hz, 1 H), 3.12-3.33 (m, 1 H), 3.60-3.74 (m, 1 H), 3.79-4.04 (m, 5 H), 6.79-7.51 (m, 5 NMR (101 MHz, CHLOROFORM-d) δ ppm 29.26 (s, 1 C), 32.83 (s, 1 C), 39.07 (s, 1 C), 51.01 (s, 1 C), 60.48 (s, 1 C), 64.52 (s, 1 C), 64.59 (s, 1 C), 72.75 (s, 1 C), 108.76 (s, 1 C), 127.28 (s, 1 C), 128.30 (s, 2 C), 128.66 (s, 2 C), 140.49 (s, 1 C).

### Step B: Preparation of (7R,8R)-7-(benzylamino)-1,4-dioxaspiro[4.5]decan-8-ol

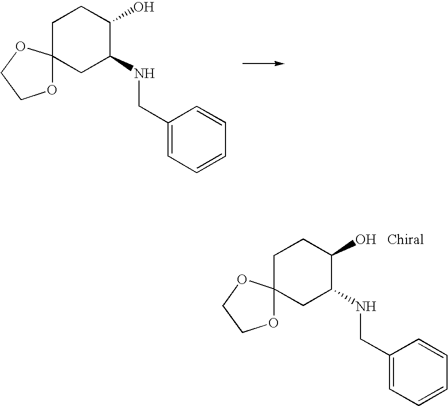

Racemate

trans-7-(benzylamino)-1,4-dioxaspiro[4.5]decan-8-ol (4.14 g, 15.72 mmol) was dissolved in a mixture of ethyl acetate (40 mL) and iPrOH (10 mL) at room temperature. D-Amygdalic acid ((R)-(−)-Mandelic acid) (1.196 g, 7.86 mmol) was added, and the resulting suspension was stirred at 80° C. for 30 minutes. The mixture was then allowed to cool to room temperature and the solid (3.09 g) was collected by filtration. The solid was recrystallized in isopropanol/MeOH (1:1, 40 mL), and then in MeOH (20 mL) to afford (R)-(−)-Mandelic acid salt of (7R,8R)-7-(benzylamino)-1,4-dioxaspiro[4.5]decan-8-ol (1.350 g, 20.67%). The absolute configuration was established by x-ray of (R)-(−)-Mandelic acid salt of (7R,8R)-7-(benzylamino)-1,4-dioxaspiro[4.5]decan-8-ol. By treating the (R)-mandelic acid salt of (7R,8R)-7-(benzylamino)-1,4-dioxaspiro[4.5]decan-8-ol with 1 N NaOH, the free base form of (7R,8R)-7-(benzylamino)-1,4-dioxaspiro[4.5]decan-8-ol was obtained. [α]_{D}²²-63.7 (c 1.31, MeOH, free base).

### Step C: Preparation of (7R,8R)-7-amino-1,4-dioxaspiro[4.5]decan-8-ol

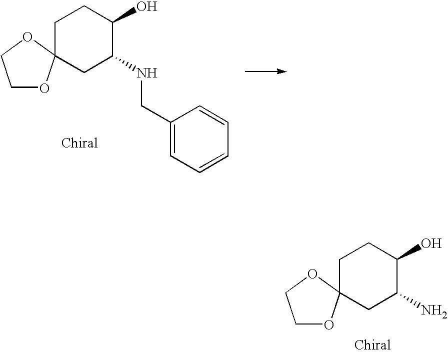

A mixture of (7R,8R)-7-(benzylamino)-1,4-dioxaspiro[4.5]decan-8-ol (2.00 g, 7.59 mmol) and 10% Pd/C (0.6 g, 0.56 mmol) in MeOH (60 mL) was hydrogenated at 40 psi of hydrogen gas atmosphere and at room temperature for 2 days. The catalysts were filtered off and the filtrate was concentrated under reduced pressure to afford the title compound (1.130 g, 86%). The crude product was used in the subsequent step without further purification. MS m/z 174.2 [M+H]⁺(ESI.

### Step D: Preparation of benzyl 4-((7R,8R)-8-hydroxy-1,4-dioxaspiro[4.5]decan-7-ylamino)piperidine-1-carboxylate

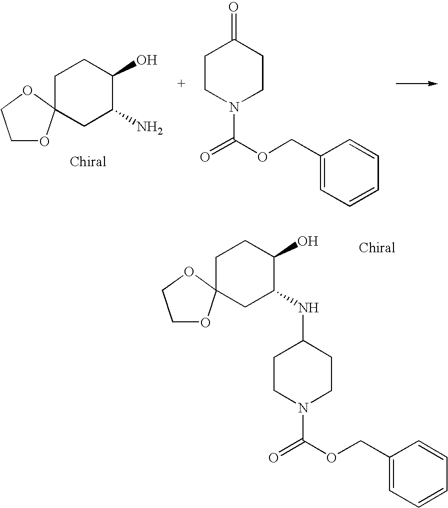

A mixture of (7R,8R)-7-amino-1,4-dioxaspiro[4.5]decan-8-ol (0.93 g, 5.37 mmol) and benzyl 4-oxopiperidine-1-carboxylate (1.252 g, 5.37 mmol) in CH₂CI₂ (30 mL) under a nitrogen atmosphere was stirred at room temperature for 30 minutes. Sodium triacetoxyborohydride (1.422 g, 6.71 mmol) was added, and the resulting mixture was stirred at room temperature for 16 hours. Saturated NaHCO₃ (20 mL) and dichloromethane (50 mL) was added to the reaction mixture, and the phases were separated. The aqueous phase was extracted with dichloromethane (3×30 mL). The combined organic extracts were washed with brine, dried over sodium sulfate, filtered and concentrated under reduced pressure. The residue was purified by flash chromatography on silica gel (dichloromethane/MeOH) to afford the title compound (1.720 g, 82%). MS m/z 391.3 [M+H]⁺(ESI).

### Step E: Preparation of benzyl 4-(2-bromo-N-((7R,8R)-8-hydroxy-1,4-dioxaspiro[4.5]decan-7-yl)acetamido)piperidine-1-carboxylate

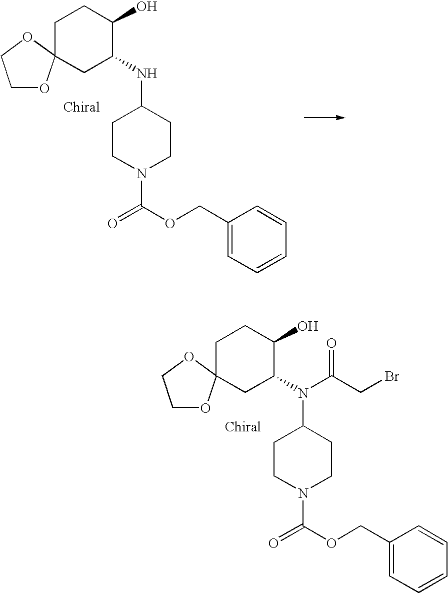

A mixture of benzyl 4-((7R,8R)-8-hydroxy-1,4-dioxaspiro[4.5]decan-7-ylamino)piperidine-1-carboxylate (1.762 g, 4.51 mmol) and N-ethyl-N-isopropylpropan-2-amine (0.8mL, 4.51 mmol) in CH₂Cl₂ (40 mL) under a nitrogen atmosphere was stirred at −45° C. for 10 minutes. A solution of 2-Bromoacetyl chloride (0.710 g, 4.51 mmol) in dichloromethane (3 mL) was added dropwise, and the resulting mixture was stirred at −45° C. for 2 hours. Saturated NaHCO₃ (10 mL) was added to the reaction mixture, and the phases were separated. The aqueous phase was extracted with EtOAc (3×50 mL). The combined organic extracts were washed with brine, 2 N HCl (10 mL), brine (10 mL), dried over sodium sulfate, filtered and concentrated under reduced pressure to give the title compound (1.85 g). The crude product was used in the subsequent step without further purification. MS m/z 511.3, 513.3 [M+H]⁺(ESI).

### Step F: Preparation of benzyl 4-((4aR,8aR)-3-oxohexahydrospiro[benzo[b][1,4]oxazine-6,2′-[1,3]dioxolane]-4(7H)-yl)piperidine-1-carboxylate

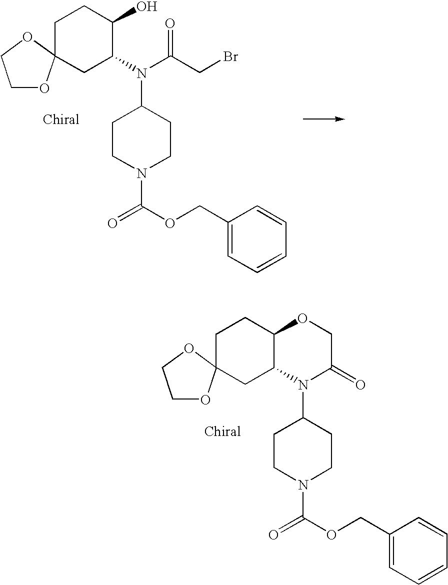

A solution of benzyl 4-(2-bromo-N-((7R,8R)-8-hydroxy-1,4-dioxaspiro[4.5]decan-7-yl)acetamido)piperidine-1-carboxylate (1.85 g) in anhydrous THF (40 mL) was cooled to −45° C. A solution of potassium 2-methylpropan-2-olate (1 M in THF, 9.02 mL, 9.02 mmol) was added in one portion to the reaction mixture. The mixture was stirred at −45° C. for 15 minutes and allowed to warm to room temperature. The reaction mixture was quenched with saturated NaHCO₃ (10 mL). EtOAc (100 mL) was added to the mixture and phases were separated. The organic phase was washed with brine (10 mL), dried over sodium sulfate, filtered and concentrated under reduced pressure. The residue was purified by flash chromatography on silica gel (EtOAc/heptane) to afford the title compound (0.450 g, 23.2%, two steps). 1H NMR (400 MHz, CHLOROFORM-D) δ ppm 1.39-1.73 (m, 6 H), 1.79 (dd, J=12.30, 2.54 Hz, 1 H), 1.87-1.99 (m, 1 H), 2.08-2.27 (m, 2 H), 2.74 (br. s., 2 H), 3.06-3.42 (m, 1 H), 3.50 (br, s.,1 H), 3.72-4.03 (m, 5 H), 4.03-4.39 (m, 4 H), 5.08 (d, J=2.34 Hz, 2 H), 6.78-7.67 (m, 5 H). MS m/z 431.3 [M+H]⁺(ESI).

### Step G: Preparation of benzyl 4-((4aR,8aR)-3,6-dioxo-2H-benzo[b][1,4]oxazin-4(3H,4aH,5H,6H,7H,8H,8aH)-yl)piperidine-1-carboxylate

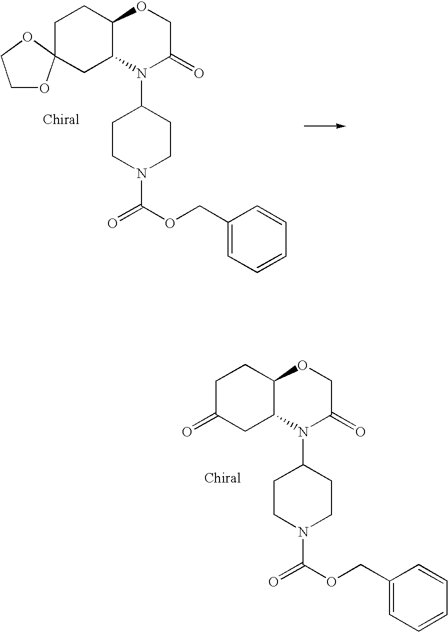

A mixture of benzyl 4-((4aR,8aR)-3-oxohexahydrospiro[benzo[b][1,4]oxazine-6,2′-[1,3]dioxolane]-4(7H)-yl)piperidine-1-carboxylate (450 mg, 1.05 mmol) and an aqueous solution of HCl (3N, 2 mL, 6.00 mmol) in THF (5 mL) under a nitrogen atmosphere was stirred at 60° C. for 1 hour. Dichloromethane (30 mL) was added to the reaction mixture, and the phases were separated. The aqueous phase was extracted with dichloromethane (3×10 mL). The combined organic extracts were washed with brine, dried over sodium sulfate, filtered and concentrated under reduced pressure. The crude product (265 mg) was used for the subsequent step without further purification. MS m/z 387.26 [M+H]⁺(ESI).

### Step H: Preparation of benzyl 4-((4aR,8aR)-6,6-difluoro-3-oxo-2H-benzo[b][1,4]oxazin-4(3H,4aH,5H,6H,7H,8H,8aH)-yl)piperidine-1-carboxylate

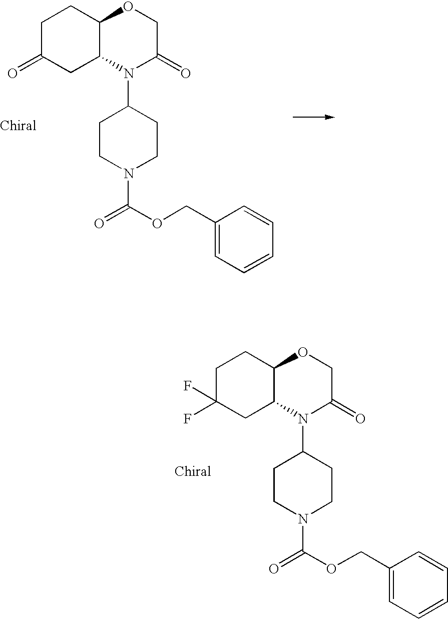

A solution of diethylamino sulfur trifluoride (240 mg, 1.49 mmol) in dichloromethane (1 mL) was added dropwise to a solution of benzyl 4-((4aR,8aR)-3,6-dioxo-2H-benzo[b][1,4]oxazin-4(3H,4aH,5H,6H,7H,8H,8aH)-yl)piperidine-1-carboxylate mg, 0.69 mmol) in CH₂Cl₂ (5 mL) at 0C. The mixture was stirred at 0° C. for 1 hour and then at room temperature for 2 hours. A solution of saturated NaHCO₃ (20 mL) was added to the mixture, stirred for 30 minutes and diluted with dichloromethane (30 mL). The organic extract was separated and the aqueous phase was washed with dichloromethane (20 mL). The combined organic extracts were washed with brine, dried over Na₂SO₄, and concentrated under reduced pressure. The residue was purified by preparative LC/MS (high pH, 40-60% Acetonitrile in water) to give the title compound (176 mg, 62.8%). 1H NMR (400 MHz, CHLOROFORM-D) δ ppm 1.36-1.93 (m, 5 H), 1.94-2.32 (m, 4 H), 2.51-2.94 (m, 3 H), 3.19-3.62 (m, 2 H), 3.65-3.95 (m,1 H), 4.00-4.43 (m, 4 H), 5.07 (br. s., 2 H), 6.87-7.59 (m, 5 H). MS m/z 409.3 [M+H]⁺(ESI).

### Step I: Preparation of (4aR,8aR)-6,6-difluoro-4-(piperidin-4-yl)hexahydro-2H-benzo[b][1,4]oxazin-3(4H)-one

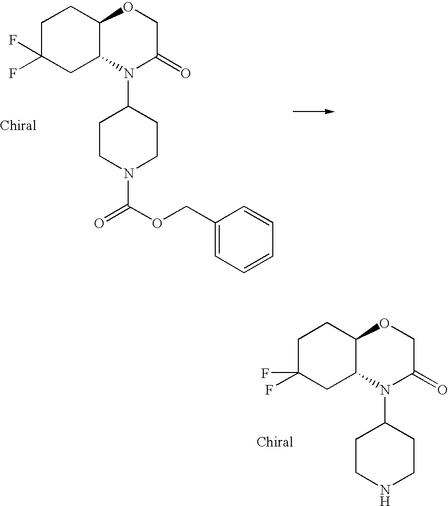

The mixture of benzyl 4-((4aR,8aR)-6,6-difluoro-3-oxo-2H-benzo[b][1,4]oxazin-4(3H,4aH,5H,6H,7H,8H,8aH)-yl)piperidine-1-carboxylate (172 mg, 0.42 mmol) an Pd/C (10%) (30 mg, 0.03 mmol) in iPrOH (30 mL) was hydrogenated at 30 psi pressure for 30 minutes. The catalyst was filtered off and the filtrate was concentrated under reduced pressure to give the title product (112 mg, 97%). The crude product was used in the subsequent step without further purification. MS m/z 275.3 [M+H]⁺(ESI).

### Intermediate 18: (4aS,8aS)-6,6-difluoro-4-(piperidin-4-yl)hexahydro-2H-benzo[b][1,4]oxazin-3(4H)-one

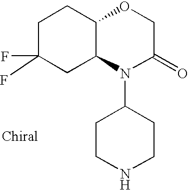

### Step A: Preparation of (7S,8S)-7-(benzylamino)-1,4-dioxaspiro[4.5]decan-8-ol

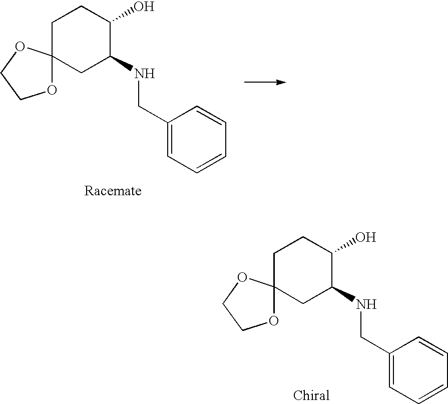

trans -7-(benzylamino)-1,4-dioxaspiro[4.5]decan-8-ol (2.63 g, 9.99 mmol) was dissolved in ethanol (40 mL) at room temperature. A solution of (S)-2-hydroxy-2-phenylacetic acid (0.760 g, 4.99 mmol) in ethanol (10 mL) was slowly added at 50° C., and the resulting suspension was stirred at 50° C. for 30 minutes and then stirred at room temperature overnight. The solid was collected and recrysallized from MeOH twice to afford the (S)-Mandelic acid salt of (7S,8S)-7-(benzylamino)-1,4-dioxaspiro[4.5]decan-8-ol (1.650 g, 39.8%). The salt was converted to its free base. [α]D22+63.9 (c 1.05, MeOH).

### Step B: Preparation of 7S,8S)-7-amino-1,4-dioxaspiro[4.5]decan-8-ol

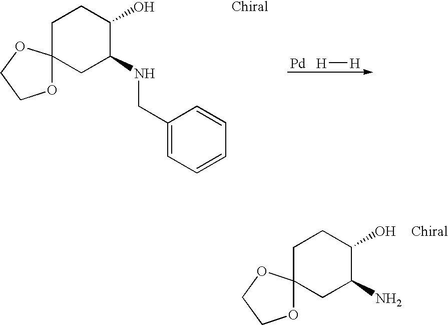

A mixture of (7S,8S)-7-(benzylamino)-1,4-dioxaspiro[4.5]decan-8-ol (1.15 g, 4.37 mmol) and 10% Pd/C (0.3 g, 0.28 mmol) in MeOH (40 mL) was hydrogenated at 40 psi pressure at room temperature for 2 days. The catalysts were filtered off and the filtrate was concentrated under reduced pressure to afford the title compound (0.745 g, 98%). 1H NMR (400 MHz, CHLOROFORM-D) δ ppm 1.41 (t, J=12.30 Hz, 1 H), 1.46-1.60 (m, 2 H), 1.63-2.02 (m, 6 H), 2.56-2.75 (m, 1 H), 3.04-3.25 (m, 1 H), 3.72-4.11 (m, 4 H).

### Step C: Preparation of benzyl 4-((7S,8S)-8-hydroxy-1,4-dioxaspiro[4.5]decan-7-ylamino)piperidine-1-carboxylate

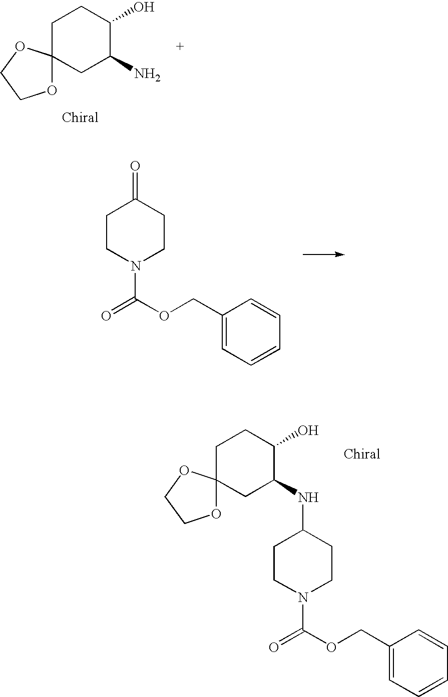

A mixture of (7S,8S)-7-amino-1,4-dioxaspiro[4.5]decan-8-ol (0.735 g, 4.24 mmol) and benzyl 4-oxopiperidine-1-carboxylate (0.990 g, 4.24 mmol) in CH₂Cl₂ (25 mL) under a nitrogen atmosphere was stirred at room temperature for 20 minutes. Sodium triacetoxyborohydride (1.124, 5.30 mmol) was added, and the resulting mixture was stirred at room temperature for 2 days. Saturated NaHCO₃ (15 mL) was added to the reaction mixture, and the phases were separated. The aqueous phase was extracted with dichloromethane (3×10 mL). The combined organic extracts were washed with brine, dried over sodium sulfate, filtered and concentrated under reduced pressure. The residue was purified by flash chromatography on silica gel, (MeOH/dichloromethane) to afford the title compound (1.313 g, 79%). MS m/z 391.3 [M+H]⁺(ESI).

### Step D: Preparation of benzyl 4-(2-bromo-N-((7S,8S)-8-hydroxy-1,4-dioxaspiro[4.5]decan-7-yl)acetamido)piperidine-1-carboxylate

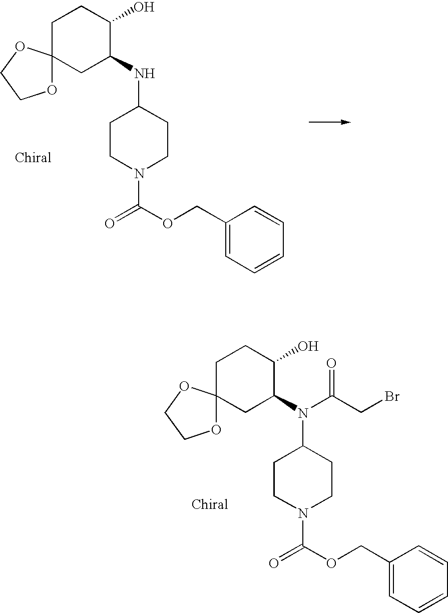

A mixture of benzyl 4-((7S,8S)-8-hydroxy-1,4-dioxaspiro[4.5]decan-7-ylamino)piperidine-1-carboxylate (1.06g, 2.71 mmol) and N,N-Diisopropylethylamine (0.529 mL, 2.99 mmol) in CH₂Cl₂ (15 mL) under a nitrogen atmosphere was stirred at −40° C. for 10 minutes. A solution of bromoacetyl chloride (0.427 g, 2.71 mmol) in CH₂Cl₂ (2 mL) was added dropwise, and the resulting mixture was stirred at −40° C. for 1 hour. A solution of HCl (1 N, 3 mL) was added to the reaction mixture, and the phases were separated. The aqueous phase was extracted with CH₂Cl₂ (3×10 mL). The combined organic extracts were washed with saturated NaHCO₃ and brine, dried over sodium sulfate, filtered and concentrated under reduced pressure to afford the title compound (1.260 g, 91%). The crude product was used in the subsequent step without further purification. MS m/z 511.2, 514.2 [M+H]⁺(ESI).

### Step E: Preparation of benzyl 4-((4aS,8aS)-3-oxohexahydrospiro[benzo[b][1,4]oxazine-6,2′-[1,3]dioxolane]-4(7H)-yl)piperidine-1-carboxylate

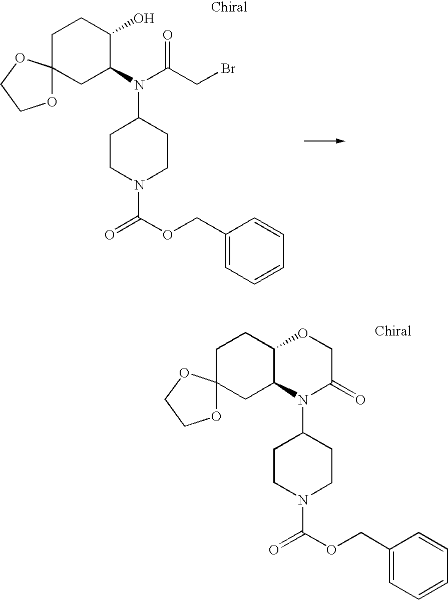

A mixture of benzyl 4-(2-bromo-N-((7S,8S)-8-hydroxy-1,4-dioxaspiro[4.5]decan-7-yl)acetamido)piperidine-1-carboxylate (1.26 g, 2.46 mmol) in THF (30 mL) under a nitrogen atmosphere was stirred at -40° C. Potassium tert-butoxide (1 M in THF) (5 mL, 5.00 mmol) was added rapidly, and the resulting mixture was stirred at −40° C. for 30 minutes. Saturated NaHCO₃ (10 mL) was added to the reaction mixture, followed by ethyl acetate (50 mL) and the phases were separated. The aqueous phase was extracted with ethyl acetate (3×30 mL). The combined organic extracts were washed with saturated NaHCO₃ and brine, dried over sodium sulfate, filtered and concentrated under reduced pressure. The residue was purified by flash chromatography on silica gel (EtOAc/heptane) to afford the title compound (0.420 g, 39.6%). 1H NMR (400 MHz, CHLOROFORM-D) δ ppm 1.46-1.72 (m, 6 H), 1.73-1.83 (m, 1 H), 1.84-1.96 (m, 1 H), 2.01-2.31 (m, 3 H), 2.73 (br. s., 2H), 3.37 (m, 1 H), 3.44-3.57 (m, 1 H), 3.72-4.02 (m, 5 H), 4.15-4.39 (m, 3 H), 5.03-5.12 (m, 2 H), 7.09-7.48 (m, 5 H). MS m/z 431.38 [M+H]⁺(ESI).

### Step F: Preparation of benzyl 4-((4aS,8aS)-3,6-dioxo-2H-benzo[b][1,4]oxazin-4(3H,4aH,5H,6H,7H,8H,8aH)-yl)piperidine-1-carboxylate

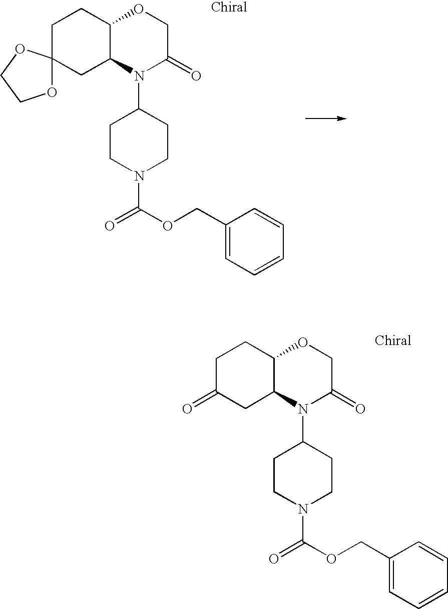

A mixture of benzyl 4-((4aS,8aS)-3-oxohexahydrospiro[benzo[b][1,4]oxazine-6,2′-[1,3]dioxolane]-4(7H)-yl)piperidine-1-carboxylate (405 mg, 0.94 mmol) and 3 N HCl aqueous solution (2 mL, 6.00 mmol) in THF (5 mL) under a nitrogen atmosphere was stirred at 60° C. for 1 hour. The mixture was allowed to cool to room temperature and diluted with dichloromethane (30 mL). Saturated NaHCO₃ (10 mL) was added to the reaction mixture, and the phases were separated. The aqueous phase was extracted with dichloromethane (3×20 mL). The combined organic extracts were washed with brine, dried over sodium sulfate, filtered and concentrated under reduced pressure. The residue was used in the subsequent step without further purification. MS m/z 387.3 [M+H]⁺(ESI).

### Step G: Preparation of benzyl 4-((4aS,8aS)-6,6-difluoro-3-oxo-2H-benzo[b][1,4]oxazin-4(3H ,4aH,5H,6H,7H,8H,8aH)-yl)piperidine-1-carboxylate

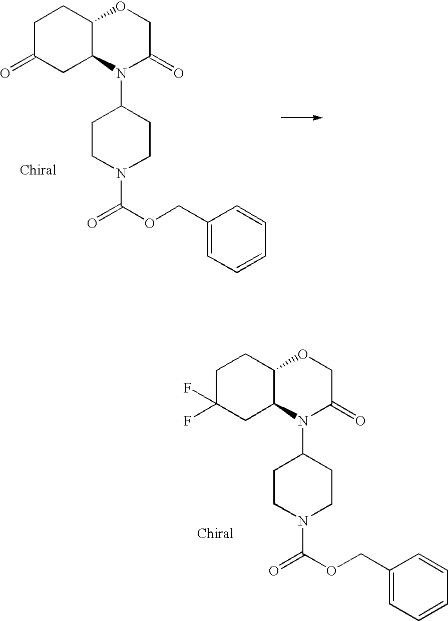

A mixture of benzyl 4-((4aS,8aS)-3,6-dioxo-2H-benzo[b][1,4]oxazin-4(3H,4aH,5H,6H,7H,8H,8aH)-yl)piperidine-1-carboxylate (320 mg, 0.83 mmol) an Diethylamino sulfur trifluoride (267 mg, 1.66 mmol) in CH₂Cl₂ (6 mL) under a nitrogen atmosphere was stirred at 0° C. for 1 hour, and then at room temperature for 2 hours. Saturated NaHCO₃ (10 mL) was added, and the resulting mixture was stirred for 30 minutes. Dichloromethane (20 mL) was added to the reaction mixture, and the phases were separated. The aqueous phase was extracted with dichloromethane (3×10 mL). The combined organic extracts were washed with brine, dried over sodium sulfate, filtered and concentrated under reduced pressure. The residue was purified by preparative LCMS (high pH, 40-60% acetonitrile in water) to afford the title compound (221 mg, 65.3%). 1H NMR (400 MHz, CHLOROFORM-D) δ ppm 1.57-1.95 (m, 5 H), 1.96-2.34 (m, 4 H), 2.55-2.87 (m, 3 H), 3.29-3.60 (m, 2 H), 3.68 (d, J=11.72 Hz, 1 H), 4.06-4.40 (m, 4 H), 5.10 (br. s., 2 H), 7.25-7.40 (m, 5 H).

### Step H: Preparation of (4aS,8aS)-6,6-difluoro-4-(piperidin-4-yl)hexahydro-2H-benzo[b][1,4]oxazin-3(4H)-one

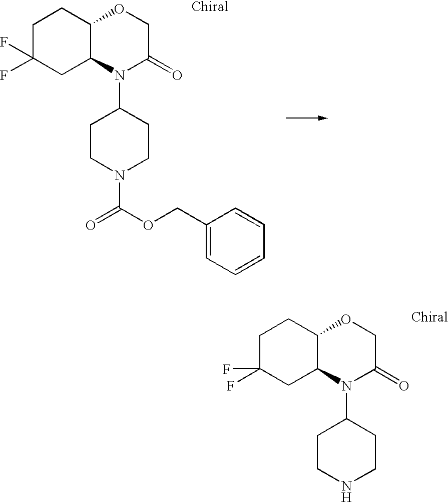

A mixture of benzyl 4-((4aS,8aS)-6,6-difluoro-3-oxo-2H-benzo[b][1,4]oxazin-4(3H,4aH,5H,6H,7H,8H,8aH)-yl)piperidine-1-carboxylate (205 mg, 0.42 mmol) an Pd/C (10%) (50 mg, 0.05 mmol) in iPrOH (60 mL) was hydrogenated at 30 psi pressure for 30 minutes. The catalyst was filtered off and the filtrate was concentrated under reduced pressure to afford the title compound (130 mg, 94%). The crude product was used in the subsequent step without purification. MS m/z 275.29 [M+H]⁺(ESI).

### EXAMPLE 1 (DIASTEREOMER 1) AND EXAMPLE 2 (DIASTEREOMER 2)

### Diastereomers of (4aR,8aS)-1-(1-(4-(propoxymethyl)cyclohexyl)piperidin-4-yl)octahydroquinazolin-2(1 H)-one

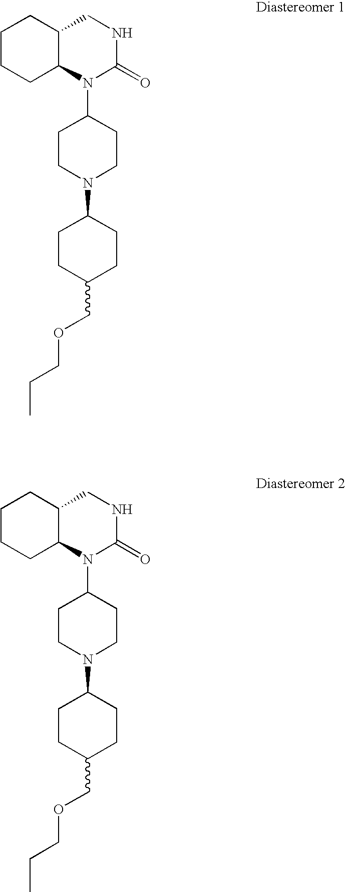

### Preparation of Diastereomers of (4aR,8aS)-1-(1-(4-(propoxymethyl)cyclohexyl)piperidin-4-yl)octahydroquinazolin-2(1 H)-one

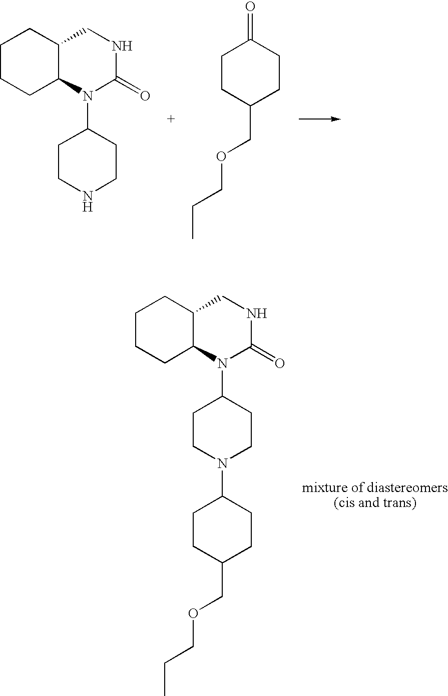

To a solution of (4aR,8aS)-1-(piperidin-4-yl)octahydroquinazolin-2(1 H)-one (HCl salt, 0.2424 g, 0.89 mmol) in MeOH (8 mL) was added microporous-carbonate resin (3.07 mmol/g, 1.2 g, 3.7 mmol) and stirred at room temperature for 1 hour. The mixture was filtered and the solid was washed well with MeOH. Filtrate was concentrated in vacuo and the residue was dissolved in dichloromethane (10 mL). 4-(propoxymethyl)cyclohexanone (0.151 g, 0.89 mmol) and acetic acid (10.14 μL, 0.18 mmol) were added to the solution. The mixture was stirred at room temperature for 40 minutes. Sodium triacetoxyborohydride (0.263 g, 1.24 mmol) was added to the mixture and stirred at room temperature for 120 hours. Saturated aqueous solution of NaHCO₃ (10 mL) was added to the mixture and loaded onto a Varian ChemElut extraction cartridge. The cartridge was washed with dichloromethane (3×12 mL). The eluant was concentrated in vacuo and the residue was purified by high pH preparative LC/MS (gradient 35-55% CH₃CN in H₂O) to provide the title compound as a mixture of diastereomers (31.0%). The mixture of diastereomers was purified by chiral supercritical fluid chromatography (Conditions: ChiralPak AS column (250×10 mm), 10 mL/minutes. Main eluent: CO₂, co-eluents: 35% (0.1% dimethylethylamine in isopropanol)) to give the corresponding two diastereomers (diastereomer 1 and diastereomer 2) of the title compound. The first eluting fraction was diastereomer 1 of the title compound (Example 1) (0.0249 g), which was obtained as a white solid. 1H NMR (400 MHz, CHLOROFORM-D) δ ppm 0.91 (t, J=7.4 Hz, 3 H), 0.95-2.57 (m, 28 H), 2.84-3.09 (m, 4 H), 3.20 (d, J=6.6 Hz, 2 H), 3.35 (t, J=6.8 Hz, 2 H), 3.68-3.97 (m, 1 H), 4.58-4.70 (m, 1 H). MS (M+1): 392.3 Exact mass calculated for C23H41N302+H: 392.3272. Found: 392.3268. The second eluting fraction was diastereomer 2 of the title compound (Example 2) (0.0597 g), which was obtained as a pale yellow solid. 1H NMR (400 MHz, CHLOROFORM-D) δ ppm 0.92 (t, J=7.4 Hz, 3 H), 1.01-1.93 (m, 23 H), 2.02-2.51 (m, 5 H), 2.85-3.12 (m, 4 H), 3.31-3.43 (m, 4 H), 3.61-3.85 (m, 1 H), 4.56-4.70 (m, 1 H). MS (M+1): 392.3. Exact mass calculated for C23H41N3O2+H: 392.3272. Found: 392.3264.

### EXAMPLE 3 (DIASTEREOMER 1) AND EXAMPEL 4 (DIASTEREOMER 2)

### Diastereomers of (4aR,8aS)-1-(1-(4-(isopropoxymethyl)cyclohexyl)piperidin-4-yl)octahydroquinazolin-2(1 H)-one

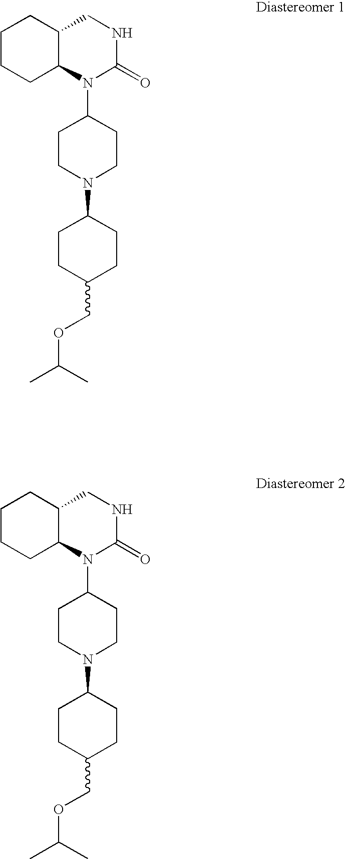

### Preparation of (4aR,8aS)-1-(1-(4-(isopropoxymethyl)cyclohexyl)piperidin-4-yl)octahydroquinazolin-2(1 H)-one

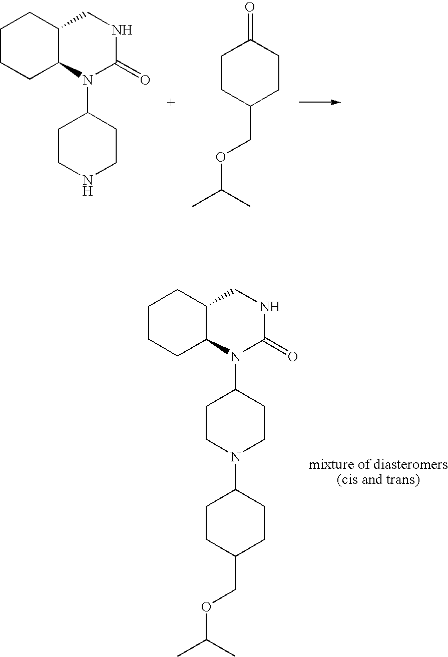

Following an analogous procedure to that described in Example 1 and Example 2, the title compound was made from (4aR,8aS)-1-(piperidin-4-yl)octahydroquinazolin-2(1H)-one (HCl salt, 0.2569 g, 0.94 mmol) and 4-(isopropoxymethyl)cyclohexanone (0.160 g, 0.94 mmol). The crude product was purified by high pH preparative LC/MS (gradient 35-55% CH₃CN in H₂O) to provide the title compound as a mixture of diastereomers (0.142 g, 38.6%). The mixture of diastereomers was purified by chiral supercritical fluid chromatography to give the corresponding two diastereomers (diastereomer 1 and diastereomer 2) of the title compound.

The first eluting fraction was diastereomer 1 of the title compound (Example 3) (0.0168 g), which was obtained as a white solid. 1H NMR (400 MHz, CHLOROFORM-D) δ ppm 0.84-1.11 (m, 3 H), 1.14 (d, J=6.2 Hz, 6 H), 1.16-1.94 (m, 18 H), 2.09-2.37 (m, 4 H), 2.41-2.51 (m, 1 H), 2.85-3.05 (m, 4 H), 3.19 (d, J=6.6 Hz, 2 H), 3.43-3.57 (m, 1 H), 3.75-3.89 (m, 1 H), 4.62 (d, J=4.7 Hz, 1 H). MS (M+1): 392.3. Exact mass calculated for C23H41N3O2+H: 392.3272. Found: 392.3266.

The second eluting fraction was diastereomer 2 of the title compound (Example 4) (0.0340 g), which was obtained as a white solid. 1H NMR (400 MHz, CHLOROFORM-D) δ ppm 0.98-1.13 (m, 1 H), 1.15 (d, J=6.2 Hz, 6 H), 1.18-1.91 (m, 20 H), 2.04-2.36 (m, 4 H), 2.37-2.49 (m, 1 H), 2.84-3.11 (m, 4 H), 3.34 (d, J=7.0 Hz, 2 H), 3.46-3.61 (m, 1 H), 3.64-3.81 (m, 1 H), 4.62 (d, J=4.3 Hz, 1 H). MS (M+1): 392.3. Exact mass calculated for C23H41 N3O2+H: 392.3272. Found: 392.3267.

### EXAMPEL 5 (DIASTEREOMER 1) AN EXAMPEL 6 (DIASTEREOMER 2)

### Diastereomers of (4aR,8aS)-1-(1-(4-propoxycyclohexyl)piperidin-4-yl)octahydroquinazolin-2(1 H)-one

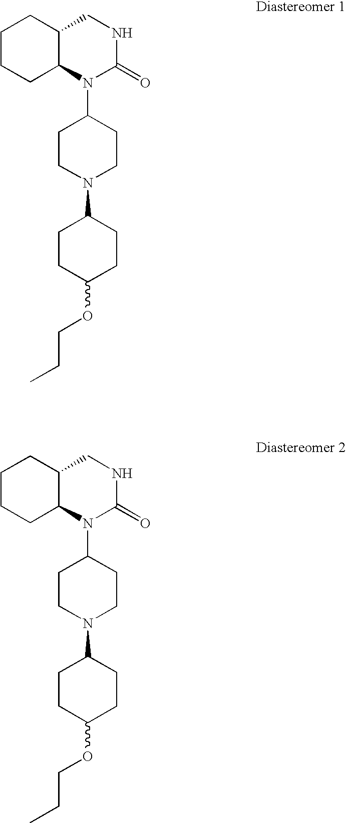

### Preparation of (4aR,8aS)-1-(1-(4-propoxycyclohexyl)piperidin-4-yl)octahydroquinazolin-2(1 H)-one

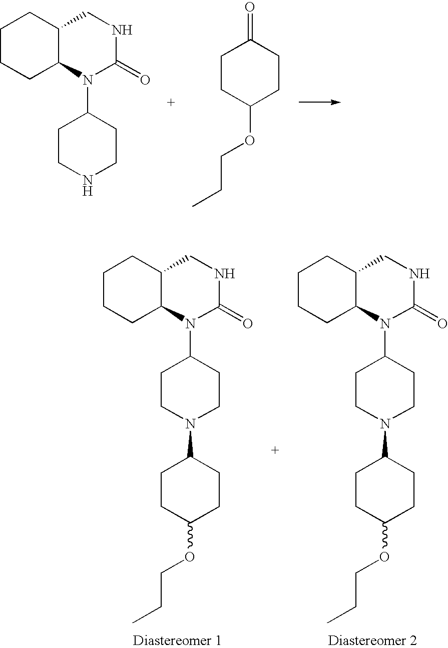

Following an analogous procedure to that described in Example 1 and Example 2, the title compound was made from (4aR,8aS)-1-(piperidin-4-yl)octahydroquinazolin-2(1H)-one (HCl salt, 0.1263 g, 0.46 mmol) and 4-propoxycyclohexanone (0.072 g, 0.46 mmol). The crude product was purified by high pH preparative LC/MS (gradient 45-65% CH₃CN in H₂O) to give the corresponding two diastereomers (diastereomer 1 and diastereomer 2) of the title compound.

The first eluting fraction was diastereomer 1 of the title compound (Example 5) (0.024 g, 13.49%), which was obtained as a white solid. 1H NMR (400 MHz, CHLOROFORM-D) δ ppm 0.75-0.85 (m, 1 H), 0.89 (t, J=7.4 Hz, 3 H), 0.98-1.41 (m, 8 H), 1.47-2.61 (m, 18 H), 2.78-3.21 (m, 5 H), 3.37 (t, J=6.6 Hz, 2 H), 3.58-3.94 (m, 1 H), 4.65 (s, 1 H). MS (M+1): 378.3. Exact mass calculated for C22H39N3O2+H: 378.3115. Found: 378.3107.

The second eluting fraction was diastereomer 2 of the title compound (Example 6) (0.020 g, 11.48%), which was obtained as a white solid. 1H NMR (400 MHz, CHLOROFORM-D) δ ppm 0.67-0.85 (m, 1 H), 0.90 (t, J=7.2 Hz, 3 H), 0.98-1.44 (m, 7 H), 1.46-2.60 (m, 19 H), 2.81-3.12 (m, 4 H), 3.30 (t, J=6.6 Hz, 2 H), 3.39-3.52 (m, 1 H), 3.65-4.06 (m, 1 H), 4.67 (d, J=2.7 Hz, 1 H). MS (M+1): 378.3. Exact mass calculated for C22H39N3O2+H: 378.3115. Found: 378.3109.

### EXAMPLE 7 (DIASTEREOMER 1)

### Diastereomer 1 of (4aR,8aS)-1-(1-(4-isopropoxycyclohexyl)piperidin-4-yl)octahydroquinazolin-2(1 H)-one

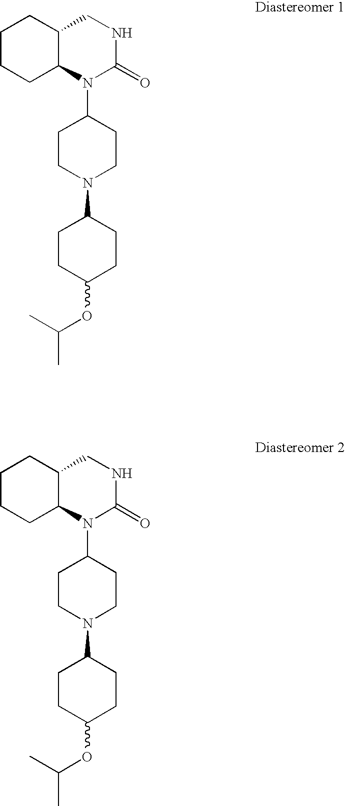

### Preparation of (4aR,8aS)-1-(1-(4-isopropoxycyclohexyl)piperidin-4-yl)octahydroquinazolin-2(1 H)-one

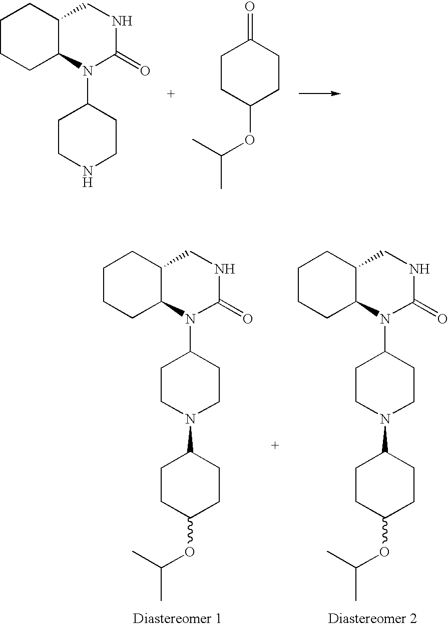

Following an analogous procedure to that described in Example 1 and Example 2, the title compound was made from (4aR,8aS)-1-(piperidin-4-yl)octahydroquinazolin-2(1 H)-one (HCl salt, 0.1207 g, 0.44 mmol) and 4-isopropoxycyclohexanone (0.069 g, 0.44 mmol). The crude product was purified by high pH preparative LC/MS (gradient 45-65% CH₃CN in H₂O) to give the corresponding two diastereomers (diastereomer 1 and diastereomer 2) of the title compound.

The first eluting fraction was diastereomer 1 of the title compound (0.0235 g, 14.12%), which was obtained as a white solid. 1H NMR (400 MHz, CHLOROFORM-D) δ ppm 0.74-1.09 (m, 3 H), 1.11 (d, J=6.2 Hz, 6 H), 1.15-1.40 (m, 6 H), 1.45-2.51 (m, 17 H), 2.80-3.06 (m, 4 H), 3.14-3.33 (m, 1 H), 3.56-3.72 (m, 1 H), 4.52-4.74 (m, 1 H). MS (M+1): 378.3. Exact mass calculated for C22H39N3O2+H: 378.3115. Found: 378.3103.

The second eluting fraction was diastereomer 2 of the title compound (Example 7) (0.0268 g, 16.10%), which was obtained as a white solid.1H NMR (400 MHz, CHLOROFORM-D) δ ppm 0.68-1.06 (m, 2 H), 1.09 (d, J=6.2 Hz, 6 H), 1.12-1.43 (m, 5 H), 1.44-2.60 (m, 18 H), 2.77-3.14 (m, 4 H), 3.46-3.65 (m, 2 H), 3.65-4.02 (m, 1 H), 4.67 (d, J=3.9 Hz, 1 H). MS (M+1): 378.3. Exact mass calculated for C22H39N3O2+H: 378.3115. Found: 378.3115. The diastereomer 2 does not show efficacy when it is tested using one or more of the biological assays described above.

### EXAMPLE 8

### (4aR,8aS)-1-(1-(4-(ethoxymethyl)cyclohexyl)piperidin-4-yl)octahydroquinazolin-2(1H)-one (mixture of diastereomers)

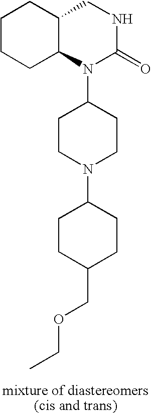

### Preparation of (4aR,8aS)-1-(1-(4-(ethoxymethyl)cyclohexyl)piperidin-4-yl)octahydroquinazolin-2(1 H)-one (mixture of diastereomers)

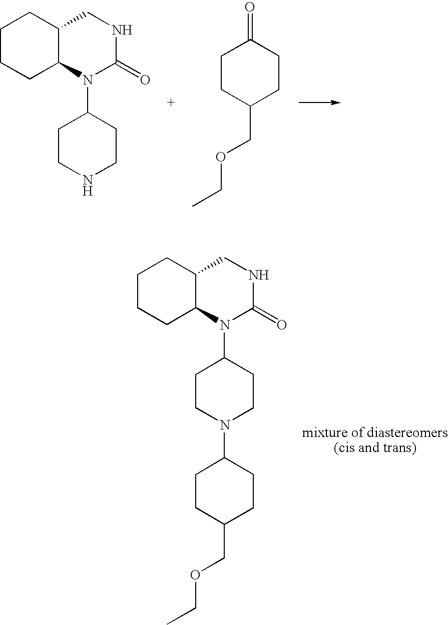

Following an analogous procedure to that described in Example 1 and Example 2, the title compound was made from (4aR,8aS)-1-(piperidin-4-yl)octahydroquinazolin-2(1 H)-one (HCl salt, 0.1248 g, 0.46 mmol) and 4-(ethoxymethyl)cyclohexanone (0.071 g, 0.46 mmol). The crude product was purified by high pH preparative LC/MS (gradient 35-55% CH₃CN in H₂O) to provide the title compound as a mixture of diastereomers (0.0344 g, 19.99%) (pale yellow solid). 1H NMR (400 MHz, CHLOROFORM-D) δ ppm 0.70-2.59 (m, 28 H), 2.77-3.12 (m, 5 H), 3.15-3.37 (m, 2 H), 3.39-3.53 (m, 2 H), 3.58-3.91 (m,1 H), 4.70 (d, J=3.9 Hz, 1 H). MS (M+1): 378.3. Exact mass calculated for C22H39N3O2+H: 378.3115. Found: 378.3121.

### EXAMPLE 9

### (4aR,8aS)-1-(1-(4-(prop-2-ynyloxy)cyclohexyl)piperidin-4-yl)octahydroquinazolin-2(1 H)-one (mixture of diastereomers)

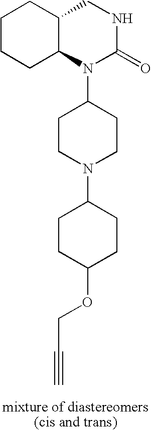

### Preparation of (4aR,8aS)-1-(1-(4-(prop-2-ynyloxy)cyclohexyl)piperidin-4-yl)octahydroquinazolin-2(1 H)-one

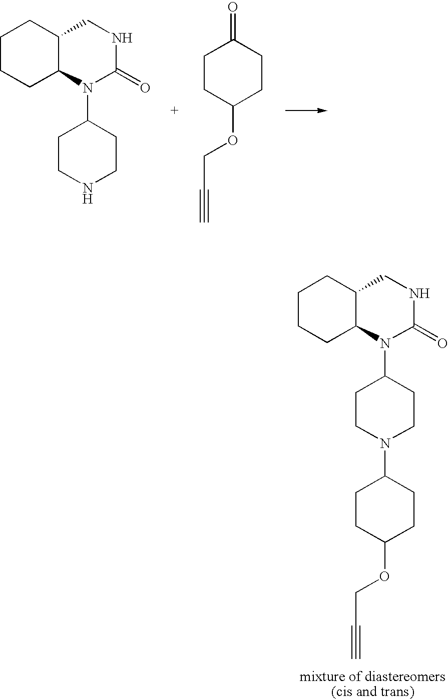

Following an analogous procedure to that described in Example 1 and Example 2, the title compound was made from (4aR,8aS)-1-(piperidin-4-yl)octahydroquinazolin-2(1 H)-one (HCl salt, 0.1221 g, 0.45 mmol) and 4-(prop-2-ynyloxy)cyclohexanone (0.068 g, 0.45 mmol). The crude product was purified by high pH preparative LC/MS (gradient 35-55% CH₃CN in H₂O) to provide the title compound as a mixture of diastereomers (pale yellow solid) (0.0398 g, 23.89%). 1H NMR (400 MHz, CHLOROFORM-D) δ ppm 0.74-2.64 (m, 25 H), 2.78-3.12 (m, 5 H), 3.33-3.89 (m, 2 H), 4.10-4.17 (m, 2 H), 4.57-4.76 (m,1 H). MS (M+1): 374.2. Exact mass calculated for C22H35N3O2+H: 374.2802. Found: 374.2802.

### TABLE 1: EXAMPLE 10-EXAMPLE 12

### All the examples in the table were made following an analogous procedure to that described in Example 1 and Example 2.

| Example | Structure | Characterization data |
|---|---|---|
| 10 | 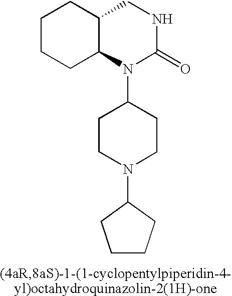 | 1H NMR (400 MHz, CHLOROFORM-D) δ ppm 0.71-2.64 (m, 24H), 2.78-3.02 (m, 3H), 3.03-3.26 (m, J = 12.1 Hz, 2H), 3.83 (s, 1H), 4.71 (d, J = 4.3 Hz, 1H). Exact mass calculated for C18H31N3O + H: 306.2540. Found: 306.2542. |
| 11 | 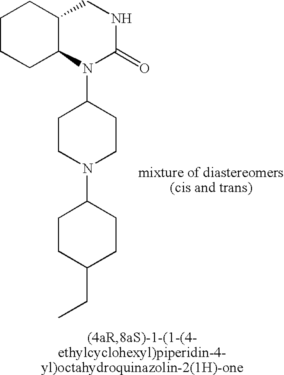 | 1H NMR (400 MHz, CHLOROFORM-D) δ ppm 0.67-0.98 (m, 4H), 0.98-1.97 (m, 22H), 1.99-3.57 (m, 10H), 4.64 (dd, J = 9.2, 4.5 Hz, 1H). Exact mass calculated for C21H37N3O + H: 348.3009. Found: 348.3005. |
| 12 | 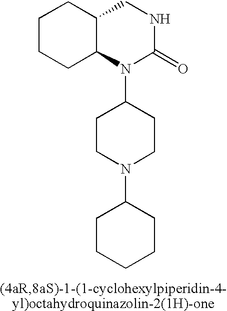 | 1H NMR (400 MHz, CHLOROFORM-D) δ ppm 0.71-1.46 (m, 10H), 1.49-2.14 (m, 14H), 2.17-2.82 (m, 4H), 2.84-3.43 (m, 4H), 4.56-4.74 (m, 1H). Exact mass calculated for C19H33N3O + H: 320.2696. Found: 320.2695. |

### EXAMPLE 13 (DIASTEREOMER 1) AND EXAMPLE 14 (DIASTEREOMER 2)

### Diastereomers of (4aS,8aS)-4-(1-(4-(ethoxymethyl)cyclohexyl)piperidin-4-yl)hexahydro-2H-benzo[b][1,4]oxazin-3(4H)-one

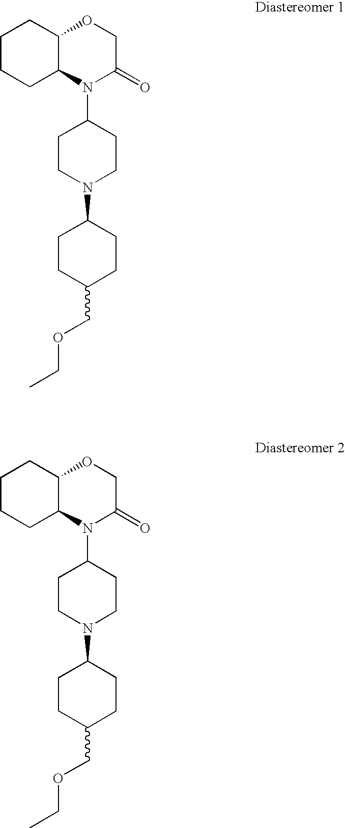

### Preparation of (4aS ,8aS)-4-(1-(4-(ethoxymethyl)cyclohexyl)piperidin-4-yl)hexahydro-2H-benzo[b][1,4]oxazin-3(4H)-one

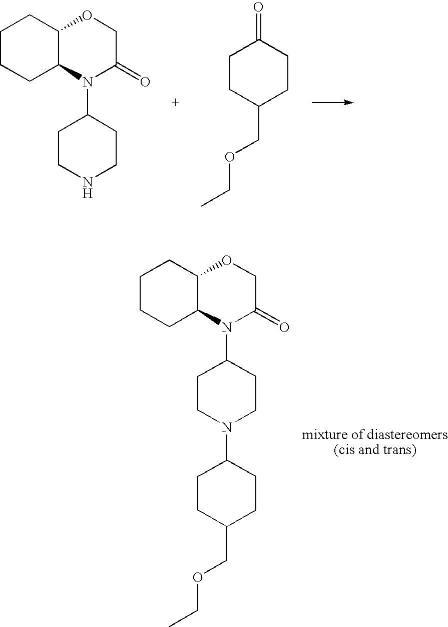

A solution of (4aS,8aS)-4-(piperidin-4-yl)hexahydro-2H-benzo[b][1,4]oxazin-3(4H)-one (HCl salt, 0.2402 g, 0.87 mmol), triethylamine (0.097 mL, 0.70 mmol), and 4-(ethoxymethyl)cyclohexanone (0.150 g, 0.96 mmol) in dichloromethane (5 mL) was stirred at room temperature for 1 hour. Sodium triacetoxyborohydride (0.278 g, 1.31 mmol) was added to the solution. The reaction mixture was stirred at room temperature for 94 hours. Saturated aqueous solution of NaHCO₃ (10 mL) was added to the mixture and loaded onto a Varian ChemElut extraction cartridge. The cartridge was washed with dichloromethane (3×12 mL). The eluant was concentrated in vacuo and the residue was purified by high pH preparative LC/MS (gradient 40-60% CH₃CN in H₂O) to provide the title compound as a mixture of diastereomers (0.083 g, 24.93%). The mixture of diastereomers was purified by chiral supercritical fluid chromatography (conditions: ChiralPak AD column (250×10 mm), 10 mL/minutes. Main eluent: CO₂, co-eluents: 55% (0.1% dimethylethylamine in Methanol) to give the corresponding two diastereomers (diastereomer 1 and diastereomer 2) of the title compound.

The first eluting fraction was diastereomer 1 of the title compound (Example 13), which was obtained as a yellow solid. 1H NMR (400 MHz, CHLOROFORM-D) δ ppm 0.80-1.08 (m, 2 H), 1.19 (t, J=7.0 Hz, 3 H), 1.22-2.57 (m, 22 H), 2.97 (br. s., 2 H), 3.15-3.33 (m, 4 H), 3.45 (q, J=7.0 Hz, 2 H), 3.99 (br. s., 1 H), 4.11-4.33 (m, 2 H). MS (M+1): 379.2. Exact mass calculated for C22H38N2O3+H: 379.2955. Found: 379.2953.

The second eluting fraction was diastereomer 2 of the title compound (Example 14), which was obtained as a yellow solid (0.0369 g, 44.5%). 1H NMR (400 MHz, CHLOROFORM-D) δ ppm 1.20 (t, J=7.0 Hz, 3 H), 1.23-1.92 (m, 17 H), 1.95-2.34 (m, 6 H), 2.39-2.51 (m, 1 H), 2.91-3.10 (m, 2 H), 3.15-3.32 (m, 2 H), 3.36 (d, J=7.0 Hz, 2 H), 3.47 (q, J=7.0 Hz, 2 H), 3.83-4.01 (m, 1 H), 4.10-4.30 (m, 2 H). MS (M+1): 379.2. Exact mass calculated for C22H38N2O3+H: 379.2955. Found: 379.2952.

### EXAMPLE 15

### (4aS,8aS)-4-(1-(4-(isopropoxymethyl)cyclohexyl)piperidin-4-yl)hexahydro-2H-benzo[b][1,4]oxazin-3(4H)-one (mixture of diastereomers)

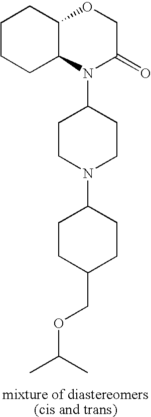

### Preparation of (4aS,8aS)-4-(1-(4-(isopropoxymethyl)cyclohexyl)piperidin-4-yl)hexahydro-2H-benzo[b][1,4]oxazin-3(4H)-one

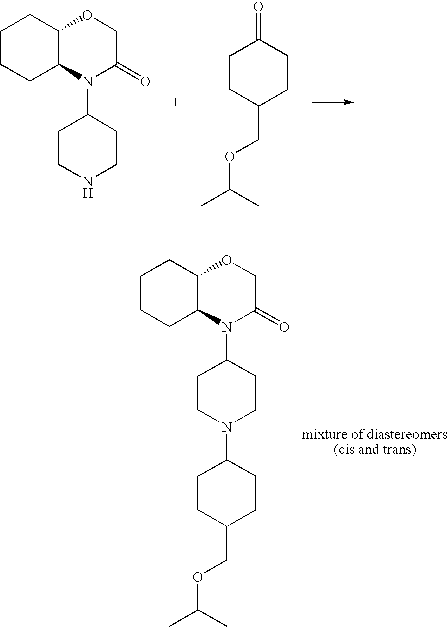

Following an analogous procedure to that described in Example 13 and Example 14, the title compound was made from (4aS,8aS)-4-(piperidin-4-yl)hexahydro-2H-benzo[b][1,4]oxazin-3(4H)-one (HCl salt, 0.2482 g, 0.90 mmol and 4-(isopropoxymethyl)cyclohexanone (0.169 g, 0.99 mmol). The crude product was purified by high pH preparative LC/MS (gradient 50-70% CH₃CN in H₂O) to provide the title compound as a mixture of diastereomers (0.236 g, 66.6%) (pale yellow solid). 1H NMR (400 MHz, CHLOROFORM-D) δ ppm 0.84-1.03 (m, 1 H), 1.09-1.18 (m, 6 H), 1.18-1.93 (m, 16 H), 1.96-2.37 (m, 6 H), 2.38-2.55 (m, 1 H), 2.87-3.10 (m, 2 H), 3.12-3.40 (m, 4 H), 3.44-3.64 (m,1 H), 3.81-4.07 (m,1 H), 4.10-4.34 (m, 2 H). MS (M+1): 393.2. Exact mass calculated for C23H40N2O3+H: 393.3112. Found: 393.3105.

### EXAMPLE 16 (DIASTEREOMER 1) AND EXAMPLE 17 (DIASTEREOMER 2)

### Diastereomers of 4aS,8aS)-4-(1-(4-propoxycyclohexyl)piperidin-4-yl)hexahydro-2H-benzo[b][1,4]oxazin-3(4H)-one

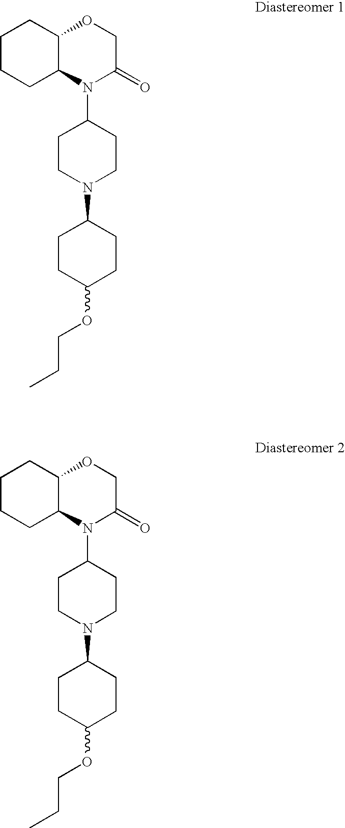

### Preparation of (4aS,8aS)-4-(1-(4-propoxycyclohexyl)piperid in-4-yl)hexahydro-2H-benzo[b][1,4]oxazin-3(4H)-one

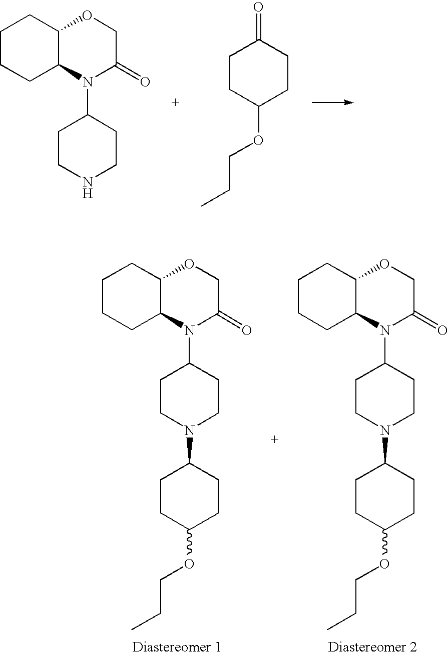

Following an analogous procedure to that described in Example 1 and Example 2, the title compound was made from (4aS,8aS)-4-(piperidin-4-yl)hexahydro-2H-benzo[b][1,4]oxazin-3(4H)-one (HCl salt, 0.1385 g, 0.50 mmol) and 4-propoxycyclohexanone (0.079 g, 0.50 mmol). The crude product was purified by high pH preparative LC/MS (gradient 45-65% CH₃CN in H₂O) to give the corresponding two diastereomers (diastereomer 1 and diastereomer 2) of the title compound.

The first eluting fraction was diastereomer 1 of the title compound (Example 16) (3.80 mg, 1.992%), which was obtained as a brown gum. 1H NMR (400 MHz, CHLOROFORM-D) δ ppm 0.72-0.85 (m, 1 H), 0.89 (t, J=7.2 Hz, 3 H), 1.03-3.06 (m, 25 H), 3.10-3.34 (m, 3 H), 3.38 (t, J=6.6 Hz, 2 H), 3.42-3.67 (m,1 H), 4.03-4.36 (m, 2 H), 4.69 (s,1 H). MS (M+1): 379.2. Exact mass calculated for C22H38N2O3+H: 379.2955. Found: 379.2954.

The second eluting fraction was diastereomer 2 of the title compound (Example 17) (8.80 mg, 4.61%), which was obtained as a brown gum. 1H NMR (400 MHz, CHLOROFORM-D) δ ppm 0.72-0.87 (m, 1 H), 0.91 (t, J=7.4 Hz, 3 H), 1.05-2.61 (m, 24 H), 2.98 (s, 2 H), 3.15-3.29 (m, 2 H), 3.31 (t, J=6.6 Hz, 2 H), 3.45 (s, 1 H), 3.99 (s, 1 H), 4.11-4.34 (m, J=16.4, 16.4, 16.4 Hz, 2 H). MS (M+1): 379.2. Exact mass calculated for C22H38N2O3+H: 379.2955. Found: 379.2960.

### EXAMPLE 18 (DIASTEREOMER 1) AND EXAMPLE 19 (DIASTEREOMER 2)

### Diastereomers of (4aS,8aS)-4-(1-(4-(isopropoxymethyl)cyclohexyl)piperidin-4-yl)hexahydro-2H-benzo[b][1,4]oxazin-3(4H)-one

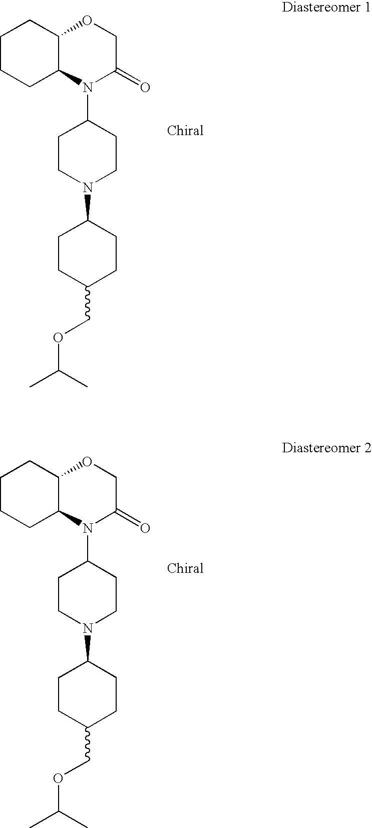

### Preparation of Diastereomer 1 and Diastereomer 2 of (4aS,8aS)-4-(1-(4-(isopropoxymethyl)cyclohexyl)piperidin-4-yl)hexahydro-2H-benzo[b][1,4]oxazin-3(4H)-one

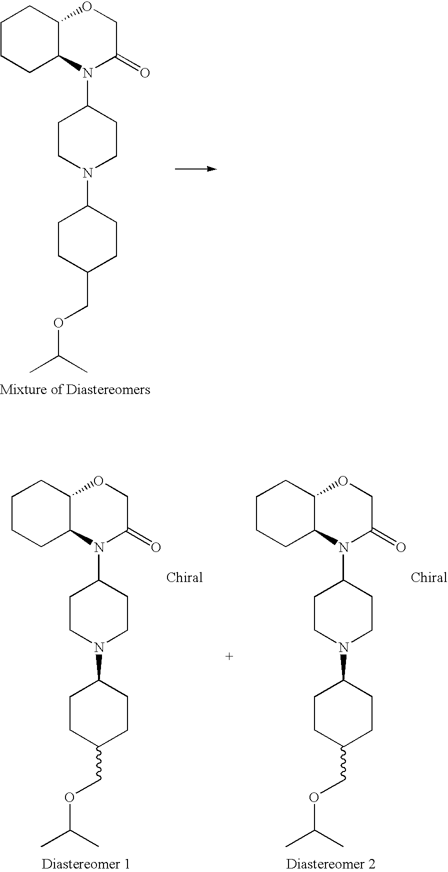

The mixture of diastereomers of (4aS,8aS)-4-(1-(4-(isopropoxymethyl)cyclohexyl)piperidin-4-yl)hexahydro-2H-benzo[b][1,4]oxazin-3(4H)-one (Example 15) (0.236 g, 0.60 mmol) was purified by SFC on a chiral stationary phase (conditions: ChiralCel AD column, 25% (iPrOH+0.1% dimethylethylamine):CO₂) to give Diastereomer 1 and Diastereomer 2 of the title compound. The first eluting diastereomer (Diastereomer 1) was further purified by high pH preparative LC/MS (gradient 50-70% CH₃CN in H₂O). (Example 18) (HCl salt, 0.082 g, 31.7%). 1H NMR (400 MHz, METHANOL-D4) δ ppm 1.16 (d, J=6.2 Hz, 6 H), 1.20-2.07 (m, 19 H), 2.31-2.49 (m, 1 H), 2.71-2.99 (m, 2 H), 3.03-4 H), 3.33-3.77 (m, 6 H), 4.14 (s, 2 H). Exact mass calculated for C23H40N2O3+H: 393.3112. Found: 393.3110.

The second eluting diastereomer (Diastereomer 2) was further purified by preparative LC/MS (gradient 50-70% CH₃CN in H₂O). (Example 19) (HCl salt, 0.030 g, 11.55%) 1H NMR (400 MHz, METHANOL-D4) δ ppm 1.13 (d, J=6.2 Hz, 6 H), 1.15-1.63 (m, 10 H), 1.76-2.19 (m, 9 H), 2.32-2.50 (m, 1 H), 2.73-2.99 (m, 2 H), 3.04 6 H), 3.44-3.60 (m, 3 H), 3.61-3.75 (m, J=12.0, 12.0, 3.7, 3.5 Hz, 1 H), 4.14 (s, 2 H). Exact mass calculated for C23H40N2O+H: 393.3112. Found: 393.3110.

### EXAMPLE 20 (DIASTEREOMER 1) AND EXAMPEL 21 (DIASTEREOMER 2)

### Diastereomers of (4aS,8aS)-4-(1-(4-(cyclopropylmethoxy)cyclohexyl)piperidin-4-yl)hexahydro-2H-benzo[b][1,4]oxazin-3(4H)-one

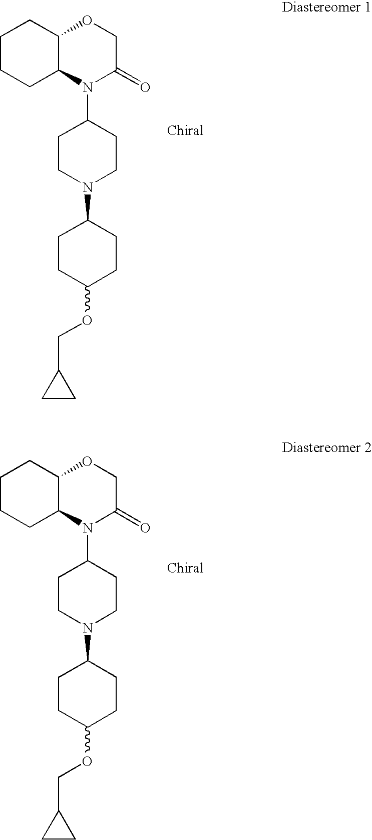

### Preparation of Diastereomer 1 and Diastereomer 2 of (4aS,8aS)-4-(1-(4-(cyclopropylmethoxy)cyclohexyl)piperidin-4-yl)hexahydro-2H-benzo[b][1,4]oxazin-3(4H)-one

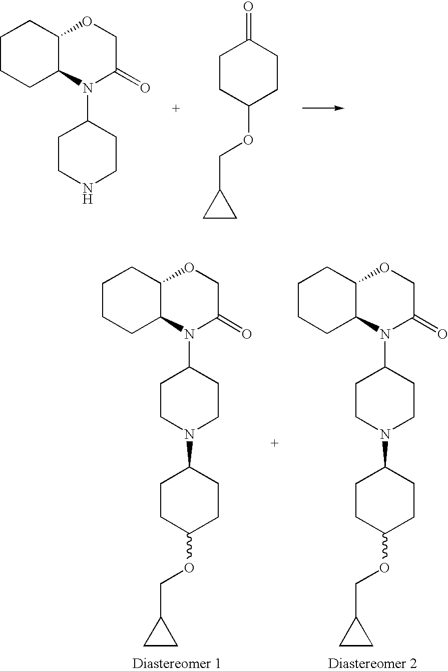

Following an analogous procedure to that described in Example 13 and Example 14, the title compound was made from (4aS,8aS)-4-(piperidin-4-yl)hexahydro-2H-benzo[b][1,4]oxazin-3(4H)-one (0.209 g, 0.76 mmol), and 4-(cyclopropylmethoxy)cyclohexanone (0.1281 g, 0.76 mmol). The crude product was purified by preparative LC/MS (gradient 45-65% CH₃CN in H₂O) to give Diastereomer 1 and Diastereomer 2 of the title compound.

The first eluting diastereomer (Diastereomer 1) (Example 20) (0.046 g, 15.47%) was obtained as a solid. 1H NMR (400 MHz, CHLOROFORM-D) δ ppm 0.13-0.22 (m, 2 H), 0.43-0.58 (m, 2 H), 0.98-1.10 (m, 1 H), 1.12-1.51 (m, 8 H), 1.62-2.37 (m, 15 H), 2.41-2.52 (m, 1 H), 2.87-3.02 (m, 2 H), 3.11-3.26 (m, 2 H), 3.28 (d, J=6.6 Hz, 2 H), 3.97 (tt, J=12.1, 3.8 Hz, 1 H), 4.13-4.31 (m, 2 H). Exact mass calculated for C23H38N2O3+H: 391.2955. Found: 391.2957.

The second eluting diastereomer (Diastereomer 2) (Example 21) (0.057 g, 19.23%) was obtained as a solid. 1H NMR (400 MHz, CHLOROFORM-D) δ ppm 0.12-0.26 (m, 2 H), 0.44-0.59 (m, 2 H), 0.97-1.10 (m, 1 H), 1.11-1.89 (m, 14 H), 1.89-2.39 (m, 8 H), 2.42-2.57 (m,1 H), 2.86-3.08 (m, 2 H), 3.13-3.38 (m, 4 H), 3.51 (qion, J=3.4 Hz, 1 H), 3.92-4.08 (m,1 H), 4.12-4.32 (m, 2 H). Exact mass calculated for C23H38N2O3+H: 391.2955. Found: 391.2950.

### EXAMPLE 22 (DIASTEREOMER 1) AND EXAMPLE 23 (DIASTEREOMER 2)

### Diastereomers of (4aS,8aS)-4-(1-(4-((cyclopropylmethoxy)methyl)cyclohexyl)piperidin-4-yl)hexahydro-2H-benzo[b][1,4]oxazin-3(4H)-one

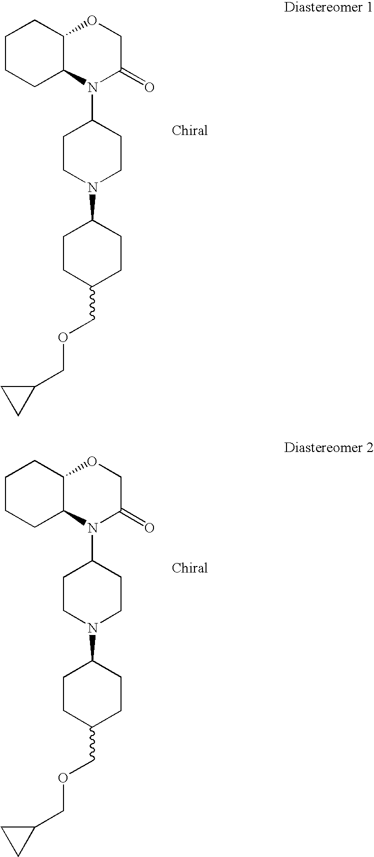

### Preparation of Diastereomer 1 and Diastereomer 2 of (4aS,8aS)-4-(1-(4-((cyclopropylmethoxy)methyl)cyclohexyl)piperidin-4-yl)hexahydro-2H-benzo[b][1,4]oxazin-3(4H)-one

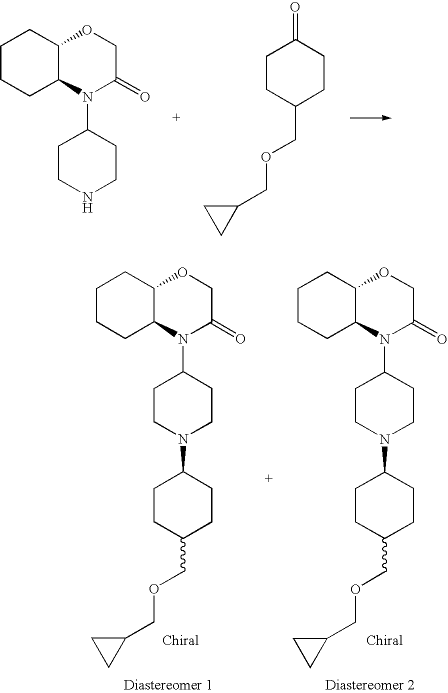

Following an analogous procedure to that described in Example 13 and Example 14, the title compound was made from (4aS,8aS)-4-(piperidin-4-yl)hexahydro-2H-benzo[b][1,4]oxazin-3(4H)-one (HCl salt) (0.114 g, 0.48 mmol), and 4-((cyclopropylmethoxy)methyl)cyclohexanone (0.0869 g, 0.48 mmol). The crude product was purified by preparative LC/MS (gradient 50-70% CH₃CN in H₂O) followed by SFC separation on a chiral stationary phase (ChiralPak AD column, 30% (iPrOH+0.1% DMEA):CO₂) to give Diastereomer 1 and Diastereomer 2 of the title compound.

The first eluting diastereomer (Diastereomer 1) (Example 22) (0.045 g, 23.48%).1H NMR (400 MHz, CHLOROFORM-D) δ ppm 0.10-0.30 (m, 2 H), 0.39-0.63 (m, 2 H), 0.97-2.34 (m, 24 H), 2.38-2.52 (m, 1 H), 2.91-3.10 (m, 2 H), 3.14-3.33 (m, 4 H), 3.38 (d, J=7.4 Hz, 2 H), 3.78-4.02 (m, 1 H), 4.08-4.34 (m, 2 H). Exact mass calculated for C24H40N2O3+H: 405.3112. Found: 405.3114.

The second eluting diastereomer (Diastereomer 2) (Example 23) (9.80 mg, 5.08%). 1H NMR (400 MHz, CHLOROFORM-D) δ ppm 0.16-0.22 (m, 2 H), 0.47-0.56 (m, 2 H), 0.83-2.37 (m, 24 H), 2.42-2.52 (m, 1 H), 2.88-3.04 (m, 2 H), 3.15-3.33 (m, 6 H), 3.99 (tt, J=12.3, 3.9 Hz, 1 H), 4.12-4.30 (m, 2 H). Exact mass calculated for C24H40N2O3+H: 405.3112. Found: 405.3108.

### EXAMPLE 24 (DIASTEREOMER 1) AND EXAMPLE 25 (DIASTEREOMER 2)

### Diastereomers of (4aS ,8aS)-4-(1-(4-((2-fluoroethoxy)methyl)cyclohexyl)piperidin-4-yl)hexahydro-2H-benzo[b][1,4]oxazin-3(4H)-one

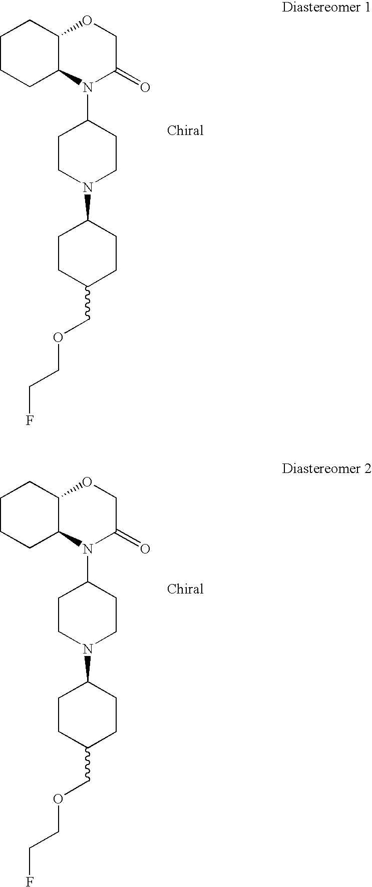

### Preparation of Diastereomer 1 and Diastereomer 2 of (4aS,8aS)-4-(1-(4-((2-fluoroethoxy)methyl)cyclohexyl)piperidin-4-yl)hexahydro-2H-benzo[b][1,4]oxazin-3(4H)-one

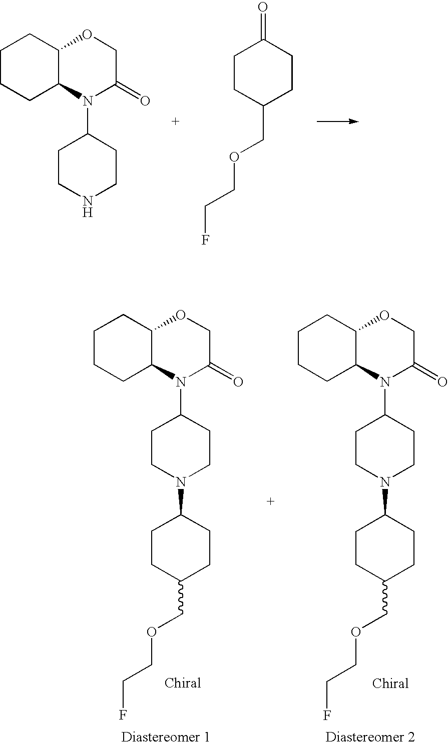

Following an analogous procedure to that described in Example 13 and Example 14, the title compound was made from (4aS,8aS)-4-(piperidin-4-yl)hexahydro-2H-benzo[b][1,4]oxazin-3(4H)-one (HCl salt) (0.111 g, 0.40 mmol) and 4-((2-fluoroethoxy)methyl)cyclohexanone (0.0703 g, 0.40 mmol). The crude product was purified by preparative LC/MS (gradient 40-60% CH₃CN in H₂O), followed by SFC on a chiral stationary phase (ChiralPak AD column, 55% (MeOH+0.1% DMEA):CO₂) to give the Diastereomer 1 and Diastereomer 2 of the title compound.

The first eluting diastereomer (Diastereomer 1) (Example 24) (0.018 g, 11.44%). 1H NMR (400 MHz, CHLOROFORM-D) δ ppm 0.87-1.05 (m, 2 H), 1.11-1.94 (m, 14 H), 1.97-2.37 (m, 7 H), 2.40-2.53 (m, 1 H), 2.86-3.04 (m, 2 H), 3.16-3.28 (m, 2 H), 3.29 (d, J=6.6 Hz, 2 H), 3.57-3.76 (m, 2 H), 3.97 (tt, J=12.3, 4.1 Hz, 1 H), 4.12-4.32 (m, 2 H), 4.44-4.65 (m, 2 H). Exact mass calculated for C22H37FN2O3+H: 397.2861. Found: 397.2860.

The second eluting diastereomer (Diastereomer 2) (Example 25) (0.038 g, 23.62%). 1H NMR (400 MHz, CHLOROFORM-D) δ ppm 1.10-2.32 (m, 23 H), 2.44 (d, J=12.5 Hz, 1 H), 2.91-3.11 (m, 2 H), 3.15-3.33 (m, 2 H), 3.44 (d, J=7.4 Hz, 2 H), 3.58-3.76 (m, 2 H), 3.82-3.96 (m, J=1 1.8, 7.7, 3.9, 3.9, 3.9 Hz, 1 H), 4.11-4.30 (m, 2 H), 4.45-4.66 (m, 2 H). Exact mass calculated for C22H37FN2O3+H: 397.2861. Found: 397.2858.

### EXAMPLE 26 (DIASTEREOMER 1) AND EXAMPLE 27 (DIASTEREOMER 2)

### Diastereomers of (4aS,8aS)-4-(1-(4-((2,2-difluoroethoxy)methyl)cyclohexyl)piperidin-4-yl)hexahydro-2H-benzo[b][1,4]oxazin-3(4H)-one

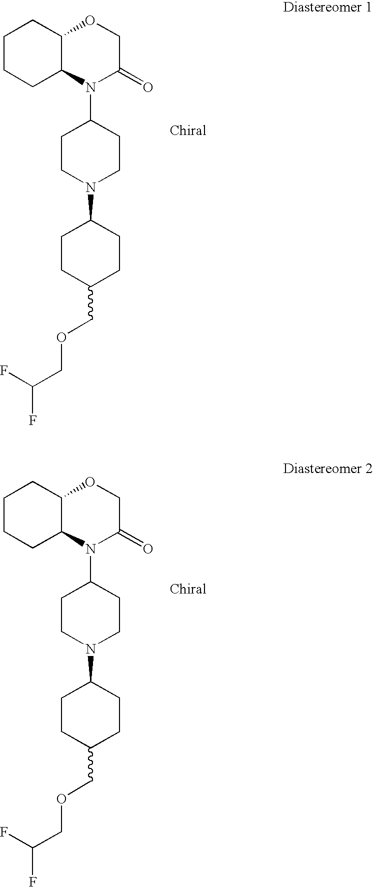

### Preparation of Diastereomer 1 and Diastereomer 2 of (4aS,8aS)-4-(1-(4-((2,2-difluoroethoxy)methyl)cyclohexyl)piperidin-4-yl)hexahydro-2H-benzo[b][1,4]oxazin-3(4H)-one

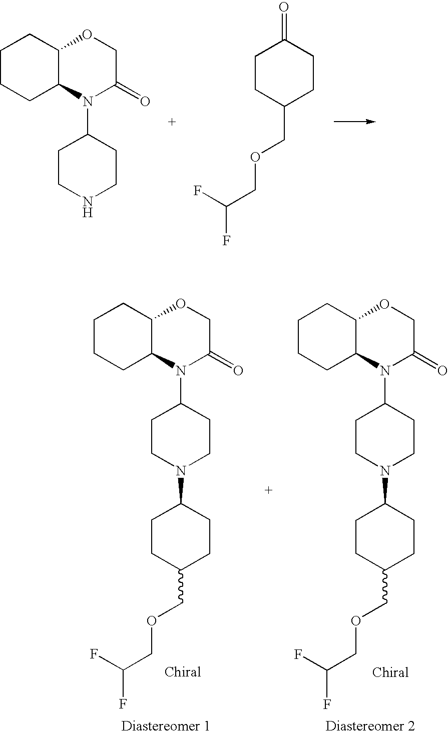

Following an analogous procedure to that described in Example 13 and Example 14, the title compound was made from (4aS,8aS)-4-(piperidin-4-yl)hexahydro-2H-benzo[b][1,4]oxazin-3(4H)-one (HCl salt) (0.218 g, 0.79 mmol), and 4-((2,2-difluoroethoxy)methyl)cyclohexanone. The crude product was purified by preparative LC/MS (gradient 50-70% CH₃CN in H₂O) followed by SFC on a chiral stationary phase (ChiralPak AD column, 55% (MeOH+0.1% DMEA):CO2) to give the Diastereomer 1 and Diastereomer 2 of the title compound.

The first eluting diastereomer (Diastereomer 1) is (Example 26). 1H NMR (400 MHz, CHLOROFORM-D) δ ppm 0.87-1.06 (m, 2 H), 1.11-1.94 (m, 15 H), 1.98-2.38 (m, 6 H), 2.47 (d, J=12.1 Hz, 1 H), 2.81-3.04 (m, 2 H), 3.14-3.31 (m, 2 H), 3.33 (d, J=6.6 Hz, 2 H), 3.63 (td, J=14.0, 4.1 Hz, 2 H), 3.98 (tt, J=12.3, 4.0 Hz, 1 H), 4.09-4.33 (m, 2 H), 5.85 (tt, J=55.6, 4.3, 4.1 Hz, 1 H). Exact mass calculated for C22H36F2N2O3+H 415.2767, found 415.2764.

The second eluting diastereomer (Diastereomer 2) (Example 27) (0.075 g, 22.73%) was obtained as a yellow gum. 1H NMR (400 MHz, CHLOROFORM-D) δ ppm 1.09-1.94 (m, 17 H), 1.97-2.33 (m, 6 H), 2.43 (dd, J=12.3, 2.5 Hz, 1 H), 2.92-3.11 (m, 2 H), 3.14-3.35 (m, 2 H), 3.47 (d, J=7.4 Hz, 2 H), 3.65 (td, J=14.1, 4.3 Hz, 2 H), 3.80-3.98 (m, 1 H), 4.10-4.32 (m, 2 H), 5.87 (tt, J=55.6, 4.1, 3.9 Hz, 1 H). Exact mass calculated for C22H36F2N2O3+H 415.2767, found 415.2769.

### EXAMPLE 28 (DIASTEREOMER 1) AND EXAMPLE 29 (DIASTEREOMER 2)

### Diastereomers of (4aS,8aS)-4-(1-(4-((cyclobutylmethoxy)methyl)cyclohexyl)piperidin-4-yl)hexahydro-2H-benzo[b][1,4]oxazin-3(4H)-one

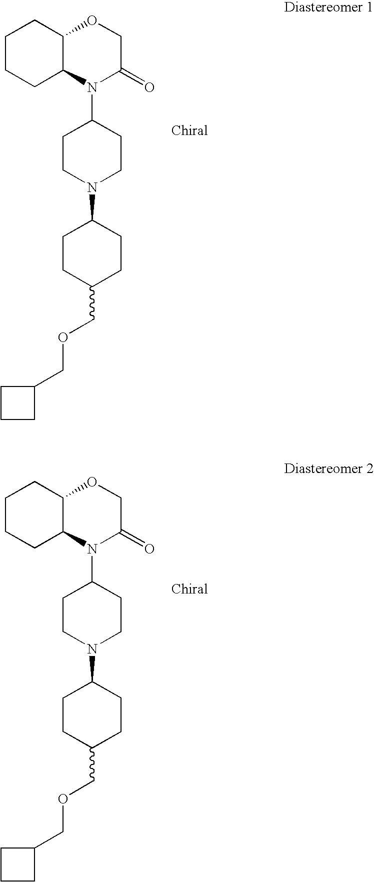

### Preparation of Diastereomer 1 and Diastereomer 2 of (4aS,8aS)-4-(1-(4-((cyclobutylmethoxy)methyl)cyclohexyl)piperidin-4-yl)hexahydro-2H-benzo[b][1,4]oxazin-3(4H)-one

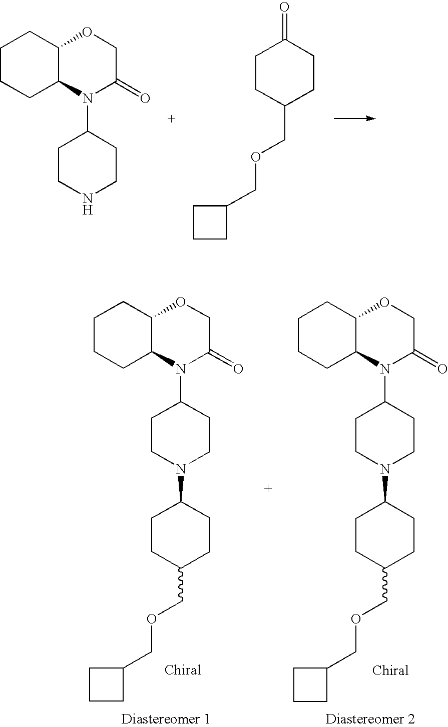

Following an analogous procedure to that described in Example 13 and Example 14, the title compound was made from (4aS,8aS)-4-(piperidin-4-yl)hexahydro-2H-benzo[b][1,4]oxazin-3(4H)-one (HCl salt) (0.134 g, 0.49 mmol), and 4-((cyclobutylmethoxy)methyl)cyclohexanone (0.0955 g, 0.49 mmol). The crude product was purified by preparative LC/MS (high pH, 60-80% acetonitrile in water), followed by SFC on a chiral stationary phase (ChiralPak AD column, 35% (iPrOH+0.1% DMEA):CO₂) to give the Diastereomer 1 and Diastereomer 2 of the title compound.

The first eluting diastereomer (Diastereomer 1) is (Example 28) (0.020 g, 9.92%). 1H NMR (400 MHz, CHLOROFORM-D) δ ppm 1.02-2.70 (m, 31 H), 2.91-3.29 (m, 4 H), 3.33 (d, J=7.4 Hz, 2 H), 3.35 (d, J=6.6 Hz, 2 H), 3.94 (br. s., 1 H), 4.09-4.28 (m, 2 H). Exact mass calculated for C25H42N2O+H 419.3268, found 419.3271.

The second eluting diastereomer (Diastereomer 2) is (Example 29) (0.013 g, 6.48%).1H NMR (400 MHz, CHLOROFORM-D) δ ppm 0.76-2.64 (m, 31 H), 3.00 (br. s., 2 H), 3.20 (d, J=6.6 Hz, 2 H), 3.23-3.33 (m, 2 H), 3.37 (d, J=7.0 Hz, 2 H), s.,1 H), 4.11-4.33 (m, 2 H). Exact mass calculated for C25H42N2O3+H 419.3268, found 419.3268.

### EXAMPLE 30 (DIASTEREOMER 1) AND EXAMPLE 31 (DIASTEREOMER 2)

### Diastereomers of (4aS,8aS)-4-(1-(4-(ethoxymethyl)-4-methylcyclohexyl)piperidin-4-yl)hexahydro-2H-benzo[b][1,4]oxazin-3(4H)-one

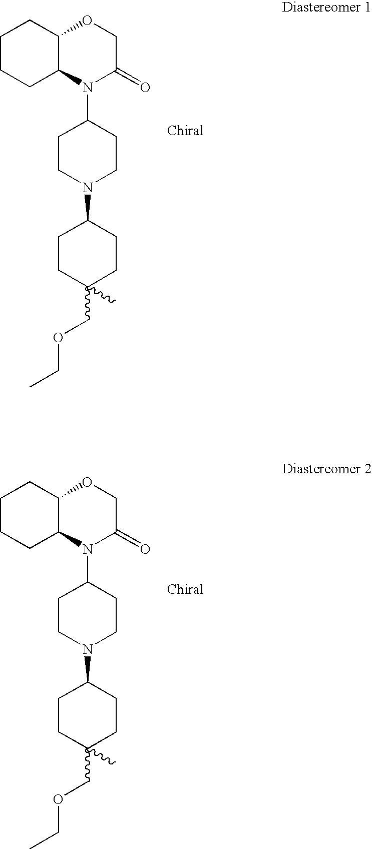

### Preparation of Diastereomer 1 and Diastereomer 2 of (4aS,8aS)-4-(l-(4-(ethoxymethyl)-4-methylcyclohexyl)piperid in-4-yl)hexahydro-2H-benzo[b][1,4]oxazin-3(4H)-one

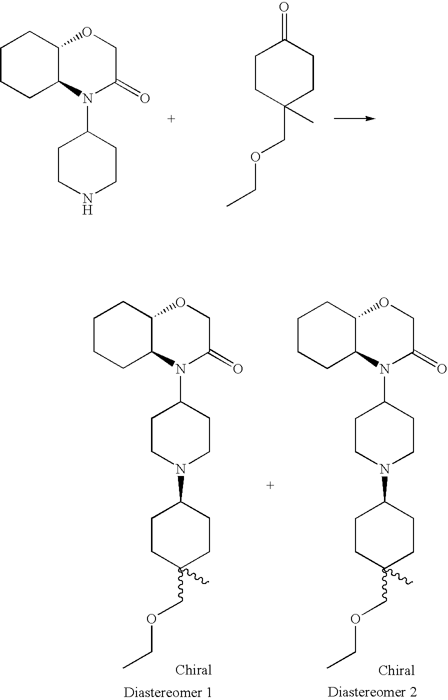

Following an analogous procedure to that described in Example 13 and Example 14, the title compound was made from (4aS,8aS)-4-(pipeid in-4-yl)hexahydro-2H-benzo[b][1,4]oxazin-3(4H)-one hydrochloride salt (0.273 g, 0.99 mmol), and 4-(ethoxymethyl)-4-methylcyclohexanone (0. 169 g, 0.99 mmol). The crude product was purified by preparative LClMS (high pH, 50-70% acetonitrile in water), followed by SFC on a chiral stationary phase (ChiralPak AD column, 40% (EtOH+0.1% DMEA):CO₂) to give the Diastereomer 1 and Diastereomer 2 of the title compound.

The first eluting diastereomer (Diastereomer 1) is (Example 30) (0.049 g, 12.65%). 1H NMR (400 MHz, CHLOROFORM-D) δ ppm 0.90 (s, 3 H), 1.02-1.92 (m, 19 H), 1.97-2.33 (m, 6 H), 2.47 (d, J=12.1 Hz, 1 H), 2.89-3.08 (m, 2 H), 3.14-3.34 (m, 4 H), 3.46 (q, J=7.0 Hz, 2 H), 3.87-4.05 (m, 1 H), 4.11-4.34 (m, 2 H). Exact mass calculated for C23H40N2O3+H 393.3112, found 393.3114.

The second eluting diastereomer (Diastereomer 2) is (Example 31) (0.027 g, 7.01%). 1H NMR (400 MHz, CHLOROFORM-D) δ ppm 0.91 (s, 3 H), 1.08-1.89 (m, 19 H), 1.97-2.37 (m, 6 H), 2.48 (d, J=12.1 Hz, 1 H), 2.90-3.04 (m, 2 H), 3.05 (s, 2 H), 3.15-3.35 (m, 2 H), 3.46 (q, J=7.0 Hz, 2 H), 3.99 (tt, J=12.3, 4.1 Hz, 1 H), 4.12-4.32 (m, 2 H). Exact mass calculated for C23H40N2O3+H 393.3112, found 393.3115.

### EXAMPLE 32

### (4aR,8aR)-4-(1-((1s,4S)-4-((cyclopropylmethoxy)methyl)cyclohexyl)piperidin-4-yl)hexahydro-2H-benzo[b][1,4]oxazin-3(4H)-one

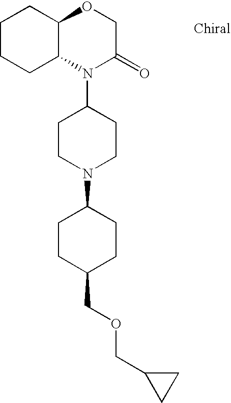

### Step A: Preparation of (4aR,8aR)-4-(1-((1s,4S)-4-((cyclopropylmethoxy)methyl)cyclohexyl)piperidin-4-yl)hexahydro-2H-benzo[b][1,4]oxazin-3(4H)-one

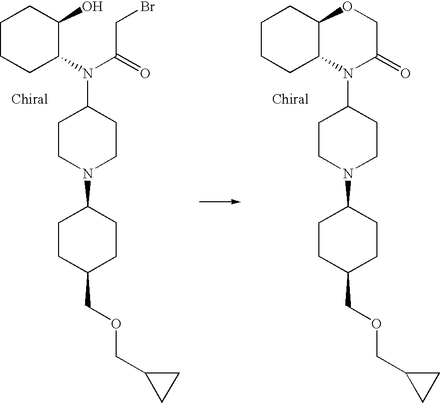

Potassium tert-butoxide (1M in THF, 2.74 ml, 2.74 mmol) was added to a solution of 2-bromo-N-(1-((1s,4S)-4-((cyclopropylmethoxy)methyl)cyclohexyl)piperidin-4-yl)-N-((1R,2R)-2-hydroxycyclohexyl)acetamide (665 mg, 1.37 mmol) in THF (11.0 ml) under nitrogen atmosphere at 0° C. The resulting mixture was stirred at 0° C. for one hour. Saturated sodium bicarbonate (10 mL) and dichloromethane (30 mL) were added and the phases were separated. The aqueous phase was extracted 3 times with dichloromethane. The combined organic extract was washed with brine, dried over sodium sulfate, filtered and concentrated under reduced pressure. The residue was purified by preparative HPLC (high pH, 50-70% acetonitrile in water) to give the title product (180 mg) as oil. 1H NMR (HCl salt) (400 MHz, CHLOROFORM-D) δ ppm 0.14-0.25 (m, 2 H), 0.48-0.58 (m, 2 H), 1.01-1.11 (m, 1 H), 1.13-1.60 (m, 10 H), 1.60-1.74 (m, 5 H), 1.75-1.92 (m, 3 H), 2.03 (d, J=10.94 Hz, 1 H), 2.07-2.30 (m, 4 H), 2.40-2.50 (m,1 H), 2.96-3.10 (m, 2 H), 3.16-3.33 (m, 4 H), 3.38 (d, J=7.42 Hz, 2 H), 3.84-3.97 (m,1 H), 4.13-4.21 (m,1 H), 4.21-4.30 (m,1 H). Exact mass calculated for C24H40N2O3 405.3112, found 405.3106.

### Step B: Preparation of 1-((1s,4s)-4-((cyclopropylmethoxy)methyl)cyclohexyl)-4,4-diethoxypiperidine

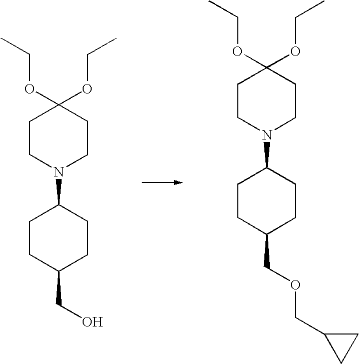

A suspension of sodium hydride (1.069 g, 26.74 mmol) in DMF (44.6 ml) was added to a solution of ((1s,4s)-4-(4,4-diethoxypiperidin-1-yl)cyclohexyl)methanol (4.24 g, 13.37 mmol) in DMF (22.28 ml) dropwise under a nitrogen atmosphere. The reaction was stirred at room temperature for 1 hour. Sodium iodide (2.004 g, 13.37 mmol) was added to the resulting mixture, followed by (bromomethyl)cyclopropane (5.00 g, 37.0 mmol). The reaction was stirred at room temperature for 5 hours. Additional amount of (bromomethyl)cyclopropane (4.30 g, 31.8 mmol) was added and the reaction was stirred at 50° C. overnight. The reaction was cooled to room temperature. Water (10 mL) was added slowly to the reaction and the mixture was concentrated under reduced pressure. The residue was dissolved in dichloromethane and saturated aqueous solution of sodium bicarbonate and the phases were separated. The aqueous phase was extracted twice with dichloromethane. The combined organic extract was washed with brine, dried over sodium sulfate, filtered and concentrated under reduced pressure. The residue was purified by flash chromatography on silica gel, (methanol/EtOAc (8-12%)) to give the title compound (2.380 g, 52.4%). 1H NMR (400 MHz, CHLOROFORM-D) δ ppm 0.15-0.26 (m, 2 H) 0.47-0.57 (m, 2 H) 1.01-1.11 (m,1 H) 1.18 (t, J=7.03 Hz, 6 H) 1.43-1.71 (m, 9 H) 1.81 (br. s., 3 H) 1.84-1.92 (m,1 H) 2.28 (br. s.,1 H) 2.54 (br. s., 4 H) 3.26 (d, J=7.03 Hz, 2 H) 3.38 (d, J=7.03 Hz, 2 H) 3.47 (q, 4 H); MS m/z 340.34 (ES+).

### Step C: Preparation of 1-((1s,4s)-4-((cyclopropylmethoxy)methyl)cyclohexyl)piperidin-4-one

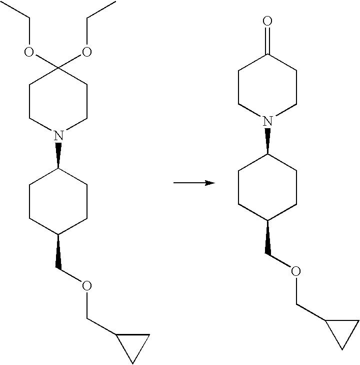

1-((1s,4s)-4-((cyclopropylmethoxy)methyl)cyclohexyl)-4,4-diethoxypiperidine (2.38 g, 7.01 mmol) was dissolved in THF (58.4 ml) and HCl (6N in water, 11.68 ml, 70.10 mmol) was added. The reaction was stirred for 1 hour at room temperature. The mixture was concentrated under reduced pressure. The residue (2.15 g) was purified by preparative HPLC (high pH, 30-50% acetonitrile in water) to give the title compound (0.500 g, 26.9%), which was used in the subsequent step without further purification. MS m/z 266.30 [M+H]+ (ES+).

### Step D: Preparation of (1R,2R)-2-(1-((1s,4S)-4-((cyclopropylmethoxy)methyl)cyclohexyl)piperidin-4-yla mino)cyclohexanol

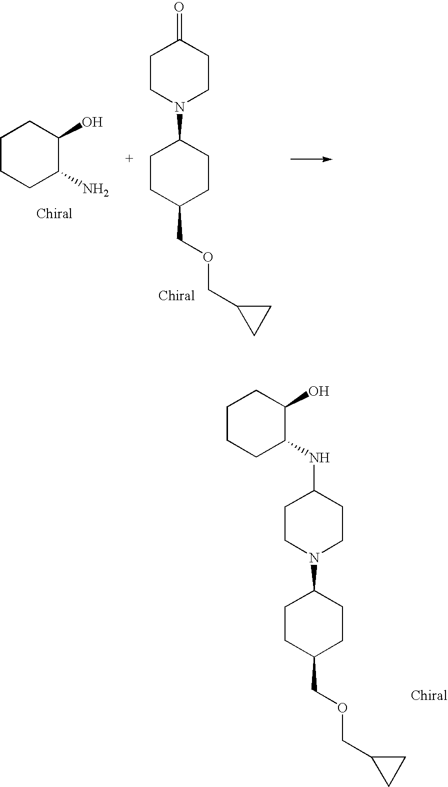

Acetic acid glacial (9.5 μl, 0.17 mmol) was added to a solution of (1R,2R)-2-aminocyclohexanol (191 mg, 1.66 mmol) and 1-((ls,4s)-4-((cyclopropylmethoxy)methyl)cyclohexyl)piperidin-4-one (500 mg, 1.66 mmol) in dichloromethane (13.100 ml) under a nitrogen atmosphere. The resulting mixture was stirred at room temperature for 4 hours. Sodium triacetoxyborohydride (527 mg, 2.48 mmol) was added and the reaction was stirred at room temperature overnight.

Solid NaHCO₃ (150 mg) was added to the reaction mixture. The mixture was stirred at room temperature for 10 minutes and concentrated under reduced pressure. The residue was purified by preparative LC/MS (high pH, 40-60% acetonitrile in water) to give the title product (500 mg, 83%). 1H NMR (400 MHz, CHLOROFORM-D) δ ppm 0.20 (q, J=4.82 Hz, 2 H), 0.47-0.58 (m, 2 H), 0.94 (br. s.,1 H), 1.06 (s, 1 H), 1.17-1.40 (m, 4 H), 1.40-1.63 (m, 7 H), 1.69 (br. s., 4 H), 1.83-2.21 (m, 9 H), 2.21-2.35 (m, 2 H), 2.58-2.69 (m, 1 H), 2.95 (d, 2 H), 3.11 (d, J=4.69 Hz, 1 H), 3.25 (d, J=6.64 Hz, 2 H), 3.38 (d, J=7.42 Hz, 2 H). MS m/z 365.26 [M+H]+ (ES+).

### Step E: Preparation of 2-bromo-N-(1-((1s,4S)-4-((cyclopropylmethoxy)methyl)cyclohexyl)piperidin-4-yl)-N-((1R,2R)-2-hydroxycyclohexyl)acetamide

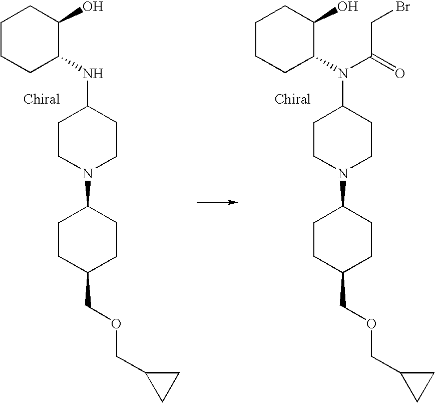

DIPEA (0.240 ml, 1.37 mmol) and 2-bromoacetyl chloride (227 mg, 1.37 mmol) were added to a solution of (1R,2R)-2-(1-((1s,4S)-4-((cyclopropylmethoxy)methyl)cyclohexyl)piperidin-4-ylamino)cyclohexanol (500 mg, 1.37 mmol) in dichloromethane (13.50 ml) at −45° C. under nitrogen atmosphere. The resulting mixture was stirred at −45° C. for 10 minutes and at room temperature for 1 hour. Ethyl acetate (25 mL) and water (5 mL) were added to the reaction mixture and the phases were separated. The organic phase was washed with brine, dried over sodium sulfate, filtered and concentrated under reduced pressure. The crude product was used in the subsequent step without purification. MS m/z 487.32 [M+H]+ (ES+).

### EXAMPLE 33 (ENANTIOMER 1) AND EXAMPLE 34 (ENANTIOMER 2)

### Enantiomers of (cis)-4-(1-((1s,4S)-4-(ethoxymethyl)cyclohexyl)piperidin-4-yl)hexahydro-2H-benzo[b][1,4]oxazin-3(4H)-one

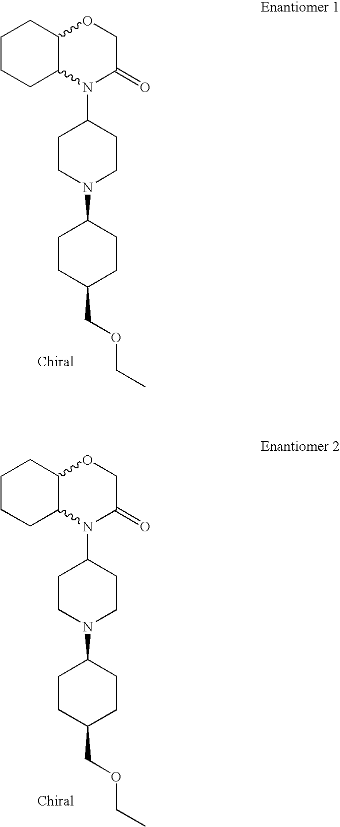

### Step A: Preparation of Enantiomer 1 and Enantiomer 2 of (cis)-4-(1-((1s,4S)-4-(ethoxymethyl)cyclohexyl)piperidin-4-yl)hexahyd ro-2H-benzo[b][1,4]oxazin-3(4H)-one

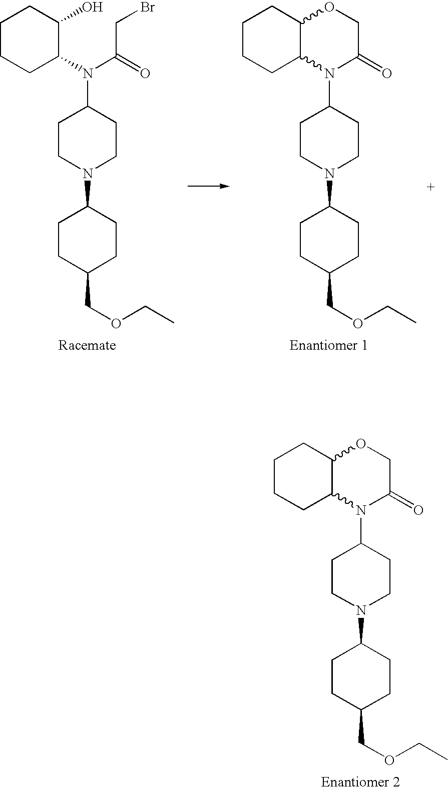

Following an analogous procedure to that described in the Step A of Example 32, the title compound was made from 2-bromo-N-(1-((1s,4S)-4-(ethoxymethyl)cyclohexyl)piperidin-4-yl)-N-((trans)-2-hydroxycyclohexyl)acetamide (1553 mg, 3.38 mmol). The crude product was purified by preparative HPLC (high pH, 50-70% acetonitrile in water) and followed by preparative LCMS (low pH, 30-50% acetonitrile in water) to give title product as a mixture of enantiomers (TFA salt, 300 mg). The enantiomers were separated by chiral preparative HPLC (ChiralPak AD column, 15:85 (Ethanol containing 0.1% diethylamine): heptane) to give Enantiomer 1 and Enantiomer 2 of the title compound.

The first eluting fraction is Enantiomer 1 (Example 33) (98 mg, 6.99%). Retention time: 15.0 minutes (ChiralPak AD column, 15:85 (Ethanol containing 0.1% diethylamine): heptane). 1H NMR (HCl salt, 400 MHz, CHLOROFORM-D) δ ppm 1.20 (t, J=7.03 Hz, 3 H), 1.38 (dd, J=12.30, 3.32 Hz, 1 H), 1.48 (t, J=7.23 Hz, 2 H), 1.51-1.64 (m, 3 H), 1.64-1.79 (m, 4 H), 1.79-1.87 (m, 1 H), 1.92-2.12 (m, 7 H), 2.69-2.96 (m, 5 H), 3.00-3.12 (m, 1 H), 3.36 (d, J=11.72 Hz, 1 H), 3.42 (d, J=7.03 Hz, 2 H), 3.48 (q, J=6.77 Hz, 2 H), 3.54 (br. s., 2 H), 3.77 (br. s.,1 H), 4.22 (d, J=17.19 Hz, 1 H), 4.34 (d, J=17.19 Hz, 1 H), 4.63 (t, J=12.30 Hz, 1 H), 11.82 (b. s., 1 H). Exact mass calculated for C22H38N2O3 379.2955 [M+H]+, found 379.2958.

The second eluting fraction is Enantiomer 2 (Example 34) 2 (110 mg, 7.84%). Retention time: 20.3 minutes (ChiralPak AD column, 15:85 (Ethanol containing 0.1% diethylamine): heptane). 1H NMR (400 MHz, CHLOROFORM-D) δ ppm 1.20 (t, 3 H), 1.36 (d, J=4.30 Hz, 1 H), 1.48 (t, J=7.23 Hz, 2 H), 1.51-1.79 (m, 7 H), 1.79-1.88 (m, 1 H), 1.91-2.12 (m, 7 H), 2.62-2.97 (m, 5 H), 2.99-3.13 (m, 1 H), 3.35 (d, J=10.55 Hz, 1 H), 3.42 (d, J=7.03 Hz, 2 H), 3.48 (q, J=7.03 Hz, 2 H), 3.55 (br. s., 2 H), 3.77 (br. s.,1 H), 4.22 (d, J=17.19 Hz, 1 H), 4.34 (d, J=17.19 Hz, 1 H), 4.63 (t, J=12.89 Hz, 1 H), 11.88 (br. s., 1 H). Exact mass calculated for C22H38N2O3 379.2955 [M+H]+, found 379.2952.

### Step B: Preparation of 4,4-diethoxy-1-((1s,4s)-4-(ethoxymethyl)cyclohexyl)piperidine

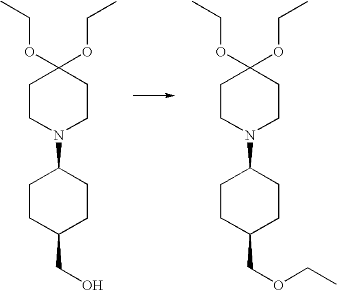

A mixture of ((1s,4s)-4-(4,4-diethoxypiperidin-1-yl)cyclohexyl)methanol (5.00 g, 17.52 mmol), iodoethane (5.60 ml, 70.07 mmol), and crushed potassium hydroxide (3.93 g, 70.07 mmol) in DMSO (38.2 ml) was stirred at room temperature for 5 days. Brine (90 mL) and diethyl ether (120 mL) were added to the reaction mixture. The layers separated, and the aqueous layer was extracted with diethyl ether (2×120 mL). The combined organic extracts was washed with brine (90 mL), dried over Na₂SO₄, and concentrated under reduced pressure to give the title compound, which was used in the subsequent step without further purification.1H NMR (400 MHz, CHLOROFORM-D) δ ppm 1.13-1.24 (m, 9 H), 1.41-1.71 (m, 8 H), 1.79 (t, J=5.47 Hz, 4 H), 1.81-1.88 (m, 1 H), 2.21-2.31 (m, 1 H), 2.52 (br. s., 4 H), 3.35 (d, J=7.42 Hz, 2 H), 3.42-3.51 (m, 6 H). MS m/z 314.35 [M+H]+ (ES+).

### Step C: Preparation of 1-((1s,4s)-4-(ethoxymethyl)cyclohexyl)piperidin-4-one

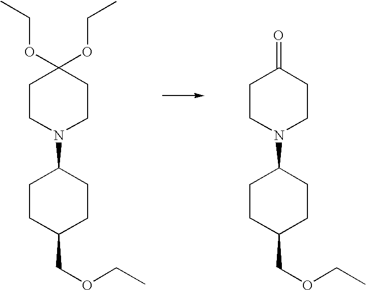

HCl (1 N in water, 20 mL, 658.24 mmol) was added to a solution of 4,4-diethoxy-1-((1s,4s)-4-(ethoxymethyl)cyclohexyl)piperidine (4.00 g, 12.76 mmol) in methanol (5 mL). The resulting mixture was stirred room temperature for 1 hour. The reaction mixture was concentrated under reduced pressure. The residue was dissolved in water and a solution of saturated sodium bicarbonate was added, followed by a solution of 1N NaOH. The aqueous phase was extracted 3 times with dichloromethane. The combined organic extract was dried over sodium sulfate, filtered and concentrated under reduced pressure. The crude product was used in the subsequent step without further purification. 1H NMR (HCl salt) (400 MHz, METHANOL-D4) δ ppm 1.13-1.23 (m, 3 H), 1.56-1.79 (m, 4 H), 1.84-2.03 (m, 7 H), 2.06-2.31 (m, 2 H), 3.03-3.29 (m, 3 H), 3.41-3.55 (m, 6 H). MS m/z 240.28 [M+H]+ (ES+).

### Step D: Preparation of (cis)-2-(1-((1s,4S)-4-(ethoxymethyl)cyclohexyl)piperidin-4-ylamino)cyclohexanol (Racemate)

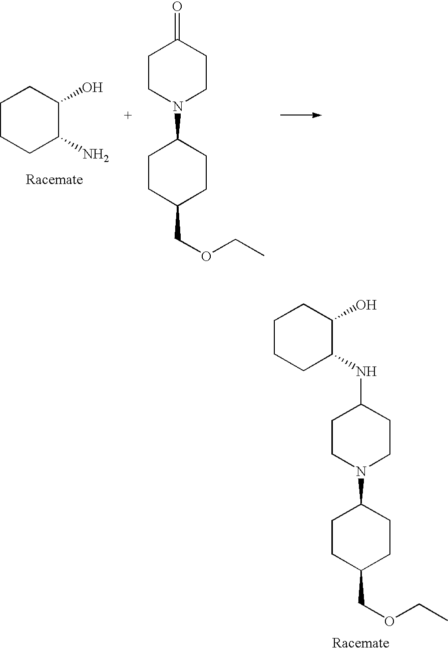

A mixture of (cis)-2-aminocyclohexanol (513 mg, 3.38 mmol) (Racemate), 1-((1s,4s)-4-(ethoxymethyl)cyclohexyl)piperidin-4-one (810 mg, 3.38 mmol) and DIPEA (0.473 ml, 2.71 mmol) in dichloromethane (26.500 ml) under a atmosphere of nitrogen was 5 stirred at room temperature overnight. Sodium triacetoxyborohydride (1076 mg, 5.08 mmol) was added to the resulting mixture and stirred room temperature for 5 days. A saturated solution of sodium bicarbonate (10 mL) and a solution of NaOH (1N, 5 mL) were added and the mixture was diluted with dichloromethane (25 mL). The aqueous phase was extracted 3 times with dichloromethane. The combined organic extract was washed with brine, dried over sodium sulfate, filtered and concentrated under reduced pressure. The crude product was used in the subsequent step without further purification. MS m/z 339.39 [M+H]+ (ES+).

### Step E: Preparation of 2-bromo-N-(1-((1s,4S)-4-(ethoxymethyl)cyclohexyl)piperidin-4-yl)-N-((trans)-2-hydroxycyclohexyl)acetamide

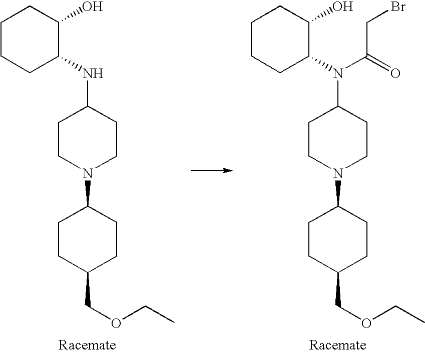

Following an analogous procedure to that described in the Step E of Example 32, the title compound was made from 2-(1-((1s,4S)-4-(ethoxymethyl)cyclohexyl)piperidin-4-ylamino)cyclohexanol. The crude product was used in the subsequent step without further purification.

### EXAMPLE 35 AND EXAMPLE 36

### (4aR,8aR)-6,6-difluoro-4-(1-(4-(isopropoxymethyl)cyclohexyl)piperidin-4-yl)hexahydro-2H-benzo[b][1,4]oxazin-3(4H)-one (Diastereomer 1, Example 35) and (4aR,8aR)-6,6-difluoro-4-(1-(4-(isopropoxymethyl)cyclohexyl)piperidin-4-yl)hexahydro-2H-benzo[b][1,4]oxazin-3(4H)-one (Diastereomer 2, Example 36)

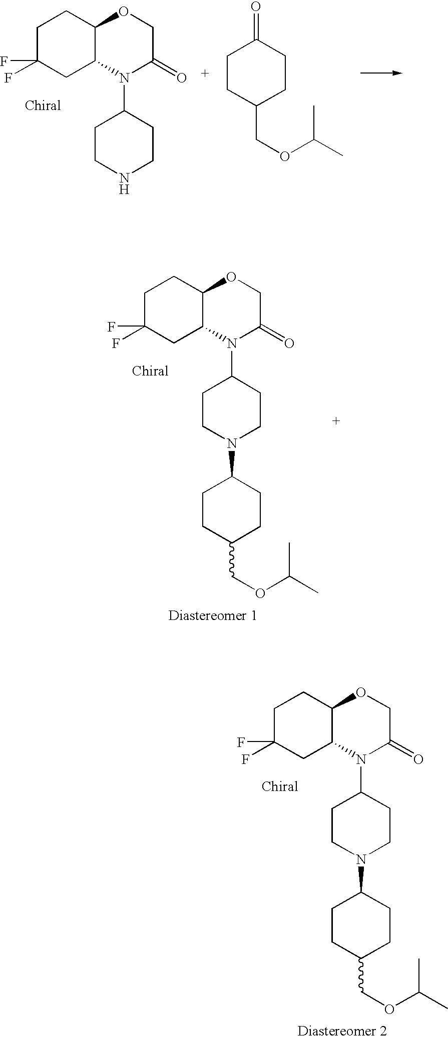

A mixture of (4aR,8aR)-6,6-difluoro-4-(piperidin-4-yl)hexahydro-2H-benzo[b][1,4]oxazin-3(4H)-one (112 mg, 0.41 mmol) and 4-(isopropoxymethyl)cyclohexanone (87 mg, 0.51 mmol) in CH₂Cl₂ (6 mL) under a nitrogen atmosphere was stirred at room temperature for 5 minutes. Sodium triacetoxyborohydride (108 mg, 0.51 mmol) was added, and the resulting mixture was stirred at room temperature overnight. Saturated NaHCO₃ (3 mL) was added to the reaction mixture, and the phases were separated. The aqueous phase was extracted with CH₂Cl₂ (3×10 mL). The combined organic extracts were washed with brine, dried over sodium sulfate, filtered and concentrated under reduced pressure. The residue was purified by preparative LCMS (high pH, 40-60% acetonitrile in water) to afford the title compound as a mixture of diastereomers (cis/trans mixture) (106 mg, 60%). The mixture of diastereomers was purified by SFC (ChiralCel OD-H, 20% MeOH with 0.1% DMEA, supercritical CO₂) to afford diastereomer 1 and diastereomer 2 of the title product.

Diastereomer 1 (Example 35) (54 mg). Retention time: 3.80 minutes. 1H NMR (400 MHz, ACETONITRILE-D3) δ ppm 1.09 (d, J=5.86 Hz, 6 H), 1.28-1.74 (m, 12 H), 1.77-2.09 (m, 4 H), 2.11-2.35 (m, 6 H), 2.62-2.79 (m, J=13.21, 6.53, 6.53, 3.08, 3.08 Hz, 1 H), 2.99 (d, J=10.94 Hz, 2 H), 3.25-3.40 (m, 3 H), 3.41-3.59 (m, 2H), 4.03-4.11 (m, 2 H). Exact mass calcuclated for C23H38F2N2O3 429.2923 [M+H]⁺, found 429.2927.

Diastereomer 2 (Example 36) (28 mg). Retention time 4.85 minutes. 1H NMR (400 MHz, ACETONITRILE-D3) δ ppm 0.84-1.00 (m, 2 H), 1.07 (d, J=5.86 Hz, 6 H), 1.15-1.31 (m, 2 H), 1.31-1.46 (m, J=15.06, 5.99, 5.99, 3.08, 3.08 Hz, 1 H), 1.47-1.69 (m, 3 H), 1.72-1.92 (m, 6 H), 1.95-2.06 (m, 2 H), 2.17-2.35 (m, 6 H), 2.64-2.77 (m, 1 H), 2.79-2.95 (m, 2 H), 3.17 (d, J=6.25 Hz, 2 H), 3.25-3.41 (m, 1 H), 3.41-3.57 (m, 2 H), 3.97-4.14 (m, 2 H). Exact mass calcuclated for C23H38F2N2O3 [M+H]⁺, found 429.2929.

### EXAMPLE 37 AND 38

### (4aS,8aS)-6,6-difluoro-4-(1-(4-(isopropoxymethyl)cyclohexyl)piperidin-4-yl)hexahydro-2H-benzo[b][1,4]oxazin-3(4H)-one (Diastereomer 1, Example 37) and (4aS,8aS)-6,6-difluoro-4-(1-(4-(isopropoxymethyl)cyclohexyl)piperidin-4-yl)hexahydro-2H-benzo[b][1,4]oxazin-3(4H)-one (Diastereomer 2, Example 38)

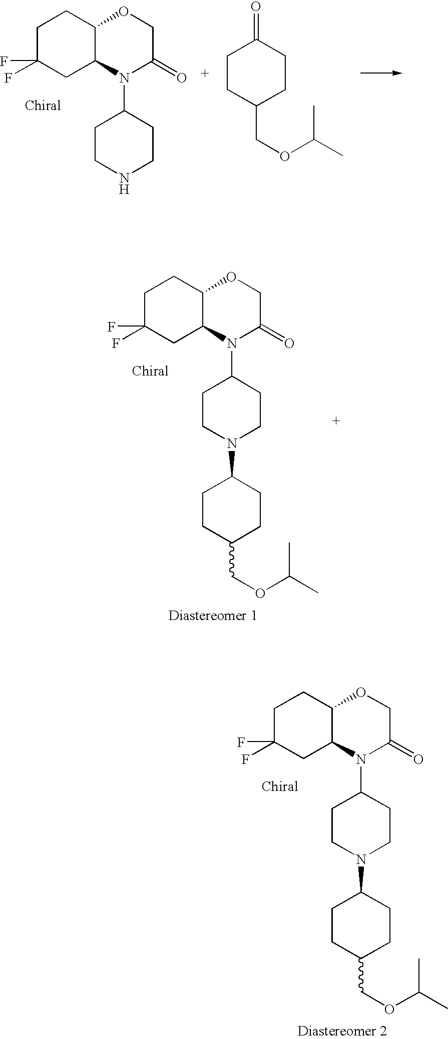

A mixture of (4aS,8aS)-6,6-difluoro-4-(piperidin-4-yl)hexahydro-2H-benzo[b][1,4]oxazin-3(4H)-one (130 mg, 0.47 mmol) and 4-(isopropoxymethyl)cyclohexanone (130 mg, 0.47 mmol) in CH₂Cl₂ (6 mL) under a nitrogen atmosphere was stirred at room temperature for 5 minutes. Sodium triacetoxyborohydride (126 mg, 0.59 mmol) was added, and the resulting mixture was stirred at room temperature overnight. Saturated NaHCO₃ (3 mL) was added to the reaction mixture, and the phases were separated. The aqueous phase was extracted with CH₂Cl₂ (3×10 mL). The combined organic extracts were washed with brine, dried over sodium sulfate, filtered and concentrated under reduced pressure. The residue was purified by preparative LCMS (high pH, 40-60% acetonitrile in water) to afford the title compound as a mixture of diastereomers (108 mg, 53%). The mixture of diastereomers was separated by SFC (ChiralPak AD-H column, 20% MeOH with 0.1% DMEA, supercritical CO₂) to afford the Diastereomer 1 and Diastereomer 2 of the title compound.

Diastereomer 1 (Example 37): (12.00 mg), Retention time: 6.63 minutes. 1H NMR (400 MHz, ACETONITRILE-D3) δ ppm 0.84-1.00 (m, 2 H), 1.07 (d, J=5.86 Hz, 6 H), 1.15-1.31 (m, 2 H), 1.31-1.46 (m, J=15.06, 5.99, 5.99, 3.08, 3.08 Hz, 1 H), 1.47-1.69 (m, 3 H), 1.72-1.92 (m, 6 H), 1.95-2.06 (m, 2 H), 2.17-2.35 (m, 6 H), 2.64-2.77 (m, 1 H), 2.79-2.95 (m, 2 H), 3.17 (d, J=6.25 Hz, 2 H), 3.25-3.41 (m, 1 H), 3.41-3.57 (m, 2 H), 3.97-4.14 (m, 2 H). MS m/z429.2 [M+H]⁺(ESI). Based on the NMR results, the substituants on the bottom hexyl ring of diastereomer 1 may have a trans configuration.

Diastereomer 2 (Example 38): (48.0 mg), Retention time 7.93 minutes. 1H NMR (400 MHz, ACETONITRILE-d₃) δ ppm 1.09 (d, J=5.86 Hz, 6 H), 1.28-1.74 (m, 12 H), 1.77-2.09 (m, 4 H), 2.11-2.35 (m, 6 H), 2.62-2.79 (m, J=13.21, 6.53, 6.53, 3.08, 3.08 Hz, 1 H), 2.99 (d, J=10.94 Hz, 2 H), 3.25-3.40 (m, 3 H), 3.41-3.59 (m, 2 H), 4.03-4.11 (m, 2 H). MS m/z 429.2 [M+H]⁺(ESI). Based on the NMR results, the substituants on the bottom hexyl ring of diastereomer 2 may have a cis configuration.

### EXAMPLE 39

### Diastereomer 2 of (4aS,8aS)-4-(1-(3-(ethoxymethyl)cyclobutyl)piperidin-4-yl)hexahydro-2H-benzo[b][1,4]oxazin-3(4H)-one

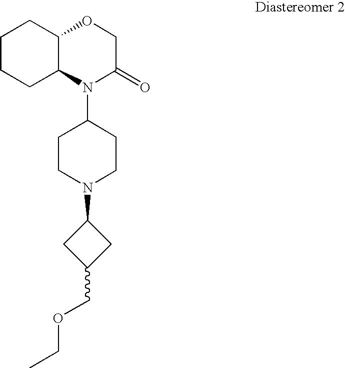

### Step A: Preparation of Diastereomer 2 of (4aS,8aS)-4-(1-(3-(ethoxymethyl)cyclobutyl)piperidin-4-yl)hexahydro-2H-benzo[b][1,4]oxazin-3(4H)-one

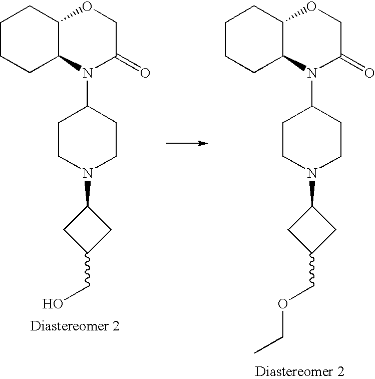

A mixture of Diastereomer 2 of (4aS,8aS)-4-(1-(3-(hydroxymethyl)cyclobutyl)piperidin-4-yl)hexahydro-2H-benzo[b][1,4]oxazin-3(4H)-one (48 mg, 0.15 mmol), potassium hydroxide (40.0 mg, 0.71 mmol) and iodoethane (0.06 mL, 0.75 mmol) in dry DMSO (5.00 mL) under a nitrogen atmosphere was stirred at room temperature for 14 hours. The mixture was lyophilized. The residue was purified by preparative HPLC (high pH, 30-50% acetonitrile in water) to afford the title product (8.8 mg, 17%) as a solid. 1H NMR (400 MHz, METHANOL-D4) δ ppm 1.16 (t, J=7.03 Hz, 3 H), 1.21-1.50 (m, 5 H), 1.55-1.70 (m, 4 H), 1.76-1.90 (m, 4 H), 1.94-2.05 (m, 1 H), 2.12-2.25 (m, 3 H), 2.31-2.48 (m, 3 H), 2.59-2.71 (m, 1 H), 2.89-3.00 (m, 2 H), 3.18-3.27 (m, 1 H), 3.18-3.27 (m, 1 H), 3.39 (d, J=5.47 Hz, 2 H), 3.48 (q, J=7.03 Hz, 1 H), 3.68 (tt, J=12.26, 3.95 Hz, 1 H), 4.12 (s, 2 H). Exact mass calculated for C20H35N2O3 351.26422 [M+H]+, found 351.25453.

### Step B: Preparation of ethyl 3-(4-((4aS,8aS)-3-oxo-2H-benzo[b][1,4]oxazin-4(3H,4aH,5H,6H,7H,8H,8aH)-yl)piperidin-1-yl)cyclobutanecarboxylate

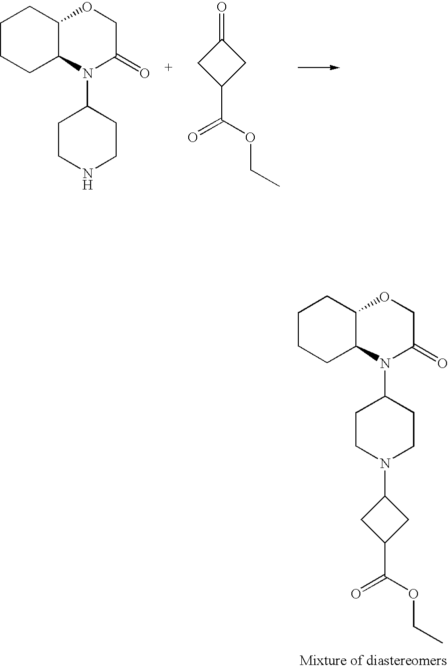

Sodium triacetoxyhydroborate (1.827 g, 8.19 mmol) was added to a mixture of (4aS,8aS)-4-(piperidin-4-yl)hexahydro-2H-benzo[b][1,4]oxazin-3(4H)-one (1.5 g, 5.46 mmol), ethyl 3-oxocyclobutanecarboxylate (0.854 g, 6.00 mmol) and triethylamine (0.913 mL, 6.55 mmol) in dichloromethane (55 mL). The resulting mixture was stirred at room temperature overnight. Saturated sodium bicarbonate (80 mL) was added to the mixture, and the phases were separated. The aqueous phase was extracted with dichloromethane (2×80 mL). The combined organic extracts were washed with brine, dried over sodium sulfate, filtered and concentrated under reduced pressure to afford the title product (2.180 g, 110%) as oil. The crude product was used in the subsequent step without further purification. MS m/z 365.4 [M+H]+ (ES+).

### Step C: Preparation of Diastereomer 2 of 3-(4-((4aS,8aS)-3-oxo-2H-benzo[b][1,4]oxazin-4(3H,4aH ,5H,6H,7H,8H,8aH)-yl)piperidin-1-yl)cyclobutanecarboxylic acid

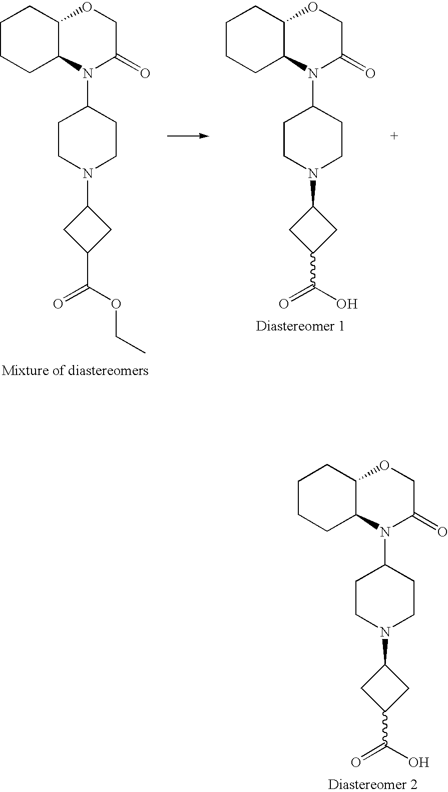

A solution of 2N sodium hydroxide (11.80 mL, 23.60 mmol) was added to a solution of ethyl 3-(4-((4aS,8aS)-3-oxo-2H-benzo[b][1,4]oxazin-4(3H,4aH,5H,6H,7H,8H,8aH)-yl)piperidin-1-yl)cyclobutanecarboxylate (1.83 g, 4.72 mmol) in methanol (55 mL).

The resulting mixture was stirred at 100° C. for 2 hours, cooled to room temperature and the volatiles were removed under reduced pressure. The remaining aqueous solution was acidified to pH 1 and then concentrated under reduced pressure. The residue was purified by preparative HPLC (high pH, 10-30% acetonitrile in water) to afford the Diastereomer 1 and Diastereomer 2 of the title product (0.746 g, 47.0%) as solids.

The first eluting diastereomer was the diastereomer 1 of the title product (not characterized). The second eluting diastereomer was the Diastereomer 2 of the title compound. 1H NMR (400 MHz, METHANOL-D4) δ ppm 1.41 (s, 4 H), 1.81 (br. s., 4 H), 1.95-2.01 (m, 1 H), 2.22-2.35 (m, 2 H), 2.35-2.53 (m, 3 H), 2.69 (br. s., 5 H), 3.30 (dt, J=3.42, 1.61 Hz, 5 H), 3.68-3.82 (m, 1 H), 4.13 (s, 2 H). MS m/z 337.3 [M+H]+ (ES+).

### Step D: Preparation of Diastereomer 2 of (4aS,8aS)-4-(1-(3-(hydroxymethyl)cyclobutyl)piperidin-4-yl)hexahydro-2H-benzo[b][1,4]oxazin-3(4H)-one

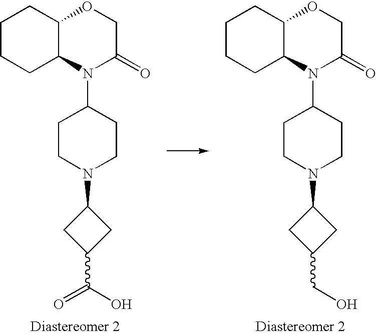

A solution of isopropyl carbonochloridate in toluene (1.886 ml, 1.89 mmol) was added to a mixture of the Diastereomer 2 of 3-(4-((4aS,8aS)-3-oxo-2H-benzo[b][1,4]oxazin-4(3H,4aH,5H,6H,7H,8H,8aH)-yl)piperidin-1-yl)cyclobutanecarboxylic acid (334 mg, 0.94 mmol) and triethylamine (0.394 ml, 2.83 mmol) in THF at 0C. The mixture was stirred at 0° C. for 60 minutes, and then a solution of sodium tetrahydroborate (143 mg, 3.77 mmol) in water (1.2 ml) was added. The reaction was stirred for 90 minutes at 0° C. and allowed to slowly warm to room temperature. Water (10 mL) was added and the mixture was extracted with CH₂Cl₂ (3×10 mL). The combined organic layers were dried over sodium sulfate, filtered and concentrated under reduced pressure. The residue was purified by preparative HPLC (high pH, 30% to 50% acetonitrile in water), to give the title product (144 mg, 47.4%) as a solid. 1H NMR (400 MHz, CHLOROFORM-D) δ ppm 1.13-1.51 (m, 4 H), 1.61-1.90 (m, 9 H), 1.98 -2.36 (m, 6 H), 2.44 (d, J=1 1.72 Hz, 1 H), 2.62 (quin, J=6.74 Hz, 1 H), 2.93-3.11 (m, 2 H), 3.17-3.33 (m, 2 H), 3.61 (d, J=4.69 Hz, 2 H), 4.08 (tt, J=12.50, 4.10 Hz, 1H), 4.14-4.30 (m, 2 H). MS m/z 323.32 [M+H]+ (ES+).

### EXAMPLE 40

### Diastereomer 2 of (4aS,8aS)-4-(1-(3-((cyclobutylmethoxy)methyl)cyclobutyl)piperidin-4-yl)hexahydro-2H-benzo[b][1,4]oxazin-3(4H)-one

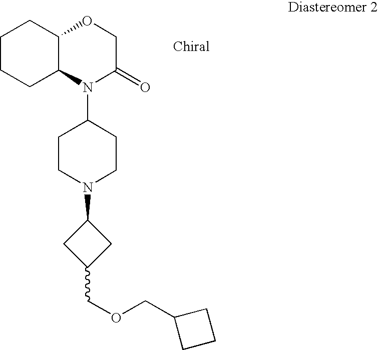

A solution of Diastereomer 2 of (4aS,8aS)-4-(1-(3-(hydroxymethyl)cyclobutyl)piperidin-4-yl)hexahydro-2H-benzo[b][1,4]oxazin-3(4H)-one (95 mg, 0.29 mmol) in DMF (3.0 mL) under a nitrogen atmosphere was stirred at 0° C. Sodium hydride (35.4 mg, 0.88 mmol) was then added and the resulting mixture was stirred at 0° C. for 20 minutes. (Bromomethyl)cyclobutane (226 mg, 1.47 mmol) was then added dropwise and the mixture was heated in the microwave at 160° C. for 10 minutes. The reaction mixture was concentrated and water (5 mL) and CH₂Cl₂ (5 mL) were added to the residue. The phases were separated and the aqueous phase was extracted with CH₂Cl₂ (2×5 mL). The combined organic extracts was washed with brine, dried over sodium sulfate, filtered and concentrated under reduced pressure. The residue was purified by preparative HPLC (high pH, 50%-70% acetonitrile in water) to give the title product (26.0 mg, 20.67%). 1H NMR (400 MHz, METHANOL-D4) δ ppm 0.83-0.93 (m, 1 H), 1.28 (s, 7 H), 1.69-2.10 (m, 11 H), 2.28-2.45 (m, 4 H), 2.68-2.92 (m, 4 H), 3.30 (dt, J=3.22, 1.71 Hz, 3 H), 3.40-3.44 (m, 3 H), 3.45-3.55 (m, 2 H), 3.64 (br. s.,1 H), 4.13 (s, 2 H). Exact mass calculated for C23H38N2O391.2955 [M+H]+, found 391.2950.

### EXAMPLE 41

### Diastereomer 2 of (4aS,8aS)-4-(1-(3-((cyclopropylmethoxy)methyl)cyclobutyl)piperidin-4-yl)hexahydro-2H-benzo[b][1,4]oxazin-3(4H)-one

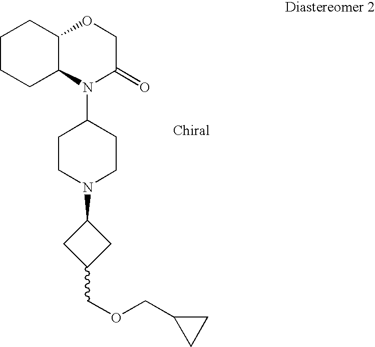

### Preparation of Diastereomer 2 of (4aS,8aS)-4-(1-(3-((cyclopropylmethoxy)methyl)cyclobutyl)piperidin-4-yl)hexahydro-2H-benzo[b][1,4]oxazin-3(4H)-one

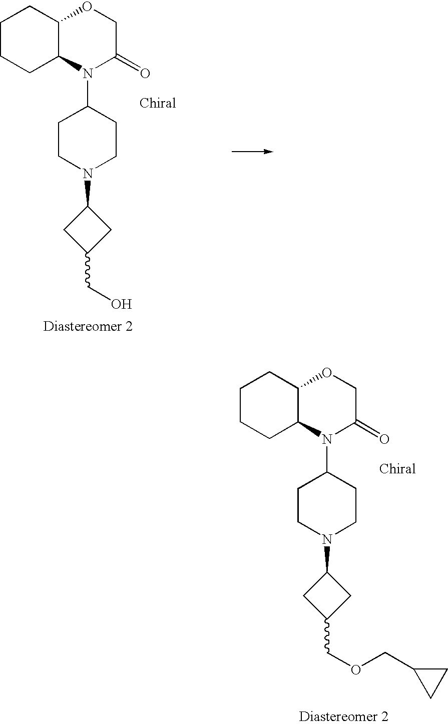

Sodium hydride (27.9 mg, 0.70 mmol) was added to a solution of Diastereomer 2 of (4aS,8aS)-4-(1-(3-(hydroxymethyl)cyclobutyl)piperidin-4-yl)hexahydro-2H-benzo[b][1,4]oxazin-3(4H)-one (45 mg, 0.14 mmol) in DMF (1.5 mL) under a nitrogen atmosphere at 0° C. The resulting mixture was stirred at 0° C. for 20 minutes. (Bromomethyl)cyclopropane (0.108 mL, 1.12 mmol) was then added dropwise and the mixture was stirred at 50° C. for 3 days. The solution was concentrated under reduced pressure and water (5 mL) and CH₂Cl₂ (5 mL) were added to the residue. The phases were separated and the aqueous phase was extracted with CH₂Cl₂ (2×5 mL). The combined organic extract was washed with brine, dried over sodium sulfate, filtered and concentrated under reduced pressure. The residue was purified by preparative HPLC (high pH, 40%-60% acetonitrile in water) to afford the title product (20.80 mg, 36.1%) as a solid. 1H NMR (400 MHz, METANOL-D4) δ ppm 0.18-0.23 (m, 2 H), 0.49-0.56 (m, 2 H), 1.00-1.11 (m, 1 H), 1.20-1.50 (m, 5 H), 1.79-1.86 (m, 2 H), 1.86-1.97 (m, 2 H), 1.96-2.08 (m, 3 H), 2.31-2.46 (m, 4 H), 2.70-2.93 (m, 4 H), 3.22-3.38 (m, 3 H), 3.43-3.56 (m, 5 H), 3.59-3.71 (m, 1 H), 4.13 (s, 2 H). Exact mass calculated for C22H36N2O3 376.2726 [M+H]+, found 377.2799.

### EXAMPLE 42

### (4aS,8aS)-4-(1-((1R,3S)-3-(ethoxymethyl)cyclopentyl)piperidin-4-yl)hexahydro-2H-benzo[b][1,4]oxazin-3(4H)-one

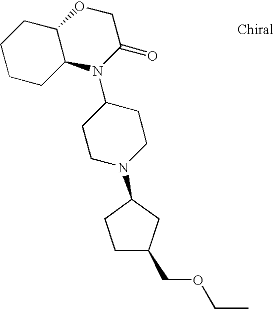

### Step A: Preparation of (4aS,8aS)-4-(1-((1R,3S)-3-(ethoxymethyl)cyclopentyl)piperidin-4-yl)hexahydro-2H-benzo[b][1,4]oxazin-3(4H)-one

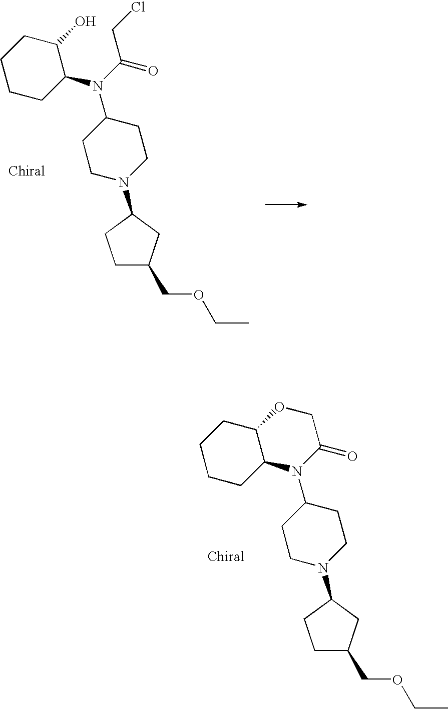

Sodium hydride (22.74 mg, 0.57 mmol) was added slowly to a solution of 2-chloro-N-(1-((1R,3S)-3-(ethoxymethyl)cyclopentyl)piperidin-4-yl)-N-((1S,2S)-2-hydroxycyclohexyl)acetamide (110 mg, 0.27 mmol) in tetrahydrofuran (10 mL). The resulting mixture was stirred at room temperature for 2 hours. The mixture was then cooled with an ice bath and quenched cautiously with a saturated solution of NH₄Cl (5 mL). EtOAc (25 mL) was added and the phases were separated. The organic layer was washed with water (2×50 mL) and brine (2×50 mL). The organic layer was dried over Na₂SO₄, filtered, and concentrated under reduced pressure. The crude product was purified by preparative LC/MS (high pH, 40-60% MeCN in water) to give the title product as a solid (HCl salt, 37.0 mg, 33.6%). 1H NMR (HCl salt) (400 MHz, CHLOROFORM-D) δ ppm 1.04 (q, J=10.94 Hz, 1 H) 1.14 (t, J=7.03 Hz, 3 H) 1.17-1.32 (m,1 H) 1.32-1.50 (m, 4 H) 1.54-1.88 (m, 6 H) 1.88-2.08 (m, 5 H) 2.08-2.28 (m, 3 H) 2.35-2.55 (m, 2 H) 3.04 (dd, J=14.45, 12.50 Hz, 2 H) 3.11-3.33 (m, 4 H) 3.42 (q, J=7.03 Hz, 2 H) 3.87-3.98 (m, 1 H) 4.06-4.25 (m, 2 H). Exact mass calculated for 365.27918 C21 H36N2O3, [M+H]+365.27987.

### Step B: Preparation of (1S,3R)-ethyl 3-(4,4-diethoxypiperidin-1-yl)cyclopentanecarboxylate

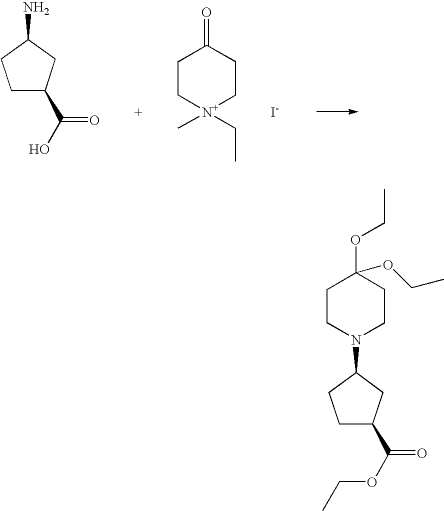

A mixture of (1S,3R)-3-aminocyclopentanecarboxylic acid (1.3 g, 10.07 mmol) and potassium carbonate (3.48 g, 25.16 mmol) in ethanol (25 mL) was heated at reflux. Asolution of 1-ethyl-1-methyl-4-oxopiperidinium iodide (4.06 g, 15.10 mmol) in water (10 mL) was added to the mixture dropwise over 10 minutes. The reaction mixture was acidified with 2N HCl until the solution reaches pH=1 and concentrated under reduced pressure. The residue was taken in ethanol (100 mL) and the solid materials were filtered off. The filtrate was concentrated under reduced pressure and the residue was taken in ethanol (100 mL). Concentrated H₂SO₄ (1 ml) was added to the mixture and heated at reflux overnight. The reaction mixture was added slowly to a saturated solution of sodium bicarbonate (200 mL). Volatiles were removed under reduced pressure and the aqueous layer was extracted with ethyl acetate (2×100 mL). Combined organic extract was dried over Na₂SO₄, filtered and concentrated under reduced pressure. The residue was purified by preparative LC/MS (high pH, 50-70% MeCN) to give the title product (0.6g, 19%). MS m/z 314.27 [M+H]+ (ES+).

### Step C: Preparation of ((1S,3R)-3-(4,4-diethoxypiperidin-1-yl)cyclopentyl)methanol

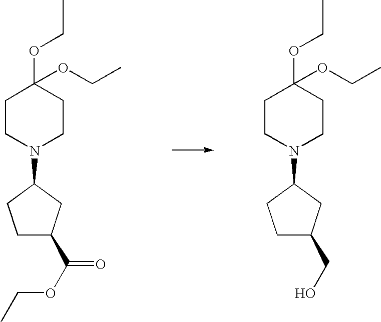

A solution of lithium aluminium hydride (2M in THF, 1.196 ml, 2.39 mmol) dropwise over 5 minutes to a solution of (1S,3R)-ethyl 3-(4,4-diethoxypiperidin-1-yl)cyclopentanecarboxylate (0.6 g, 1.91 mmol) in diethyl ether (50 mL). The mixture was stirred at room temperature for overnight. Water (0.1 mL), 15% NaOH (0.1 mL) and water (0.3 mL) were added slowly to the reaction mixture successively. Na₂SO₄ was added to the mixture, and was filtered. The solids were washed well with Et₂O, and the filtrate was concentrated under reduced pressure to give the title compound (0.51 g, 98%), which was used for the subsequent step without further purification. MS m/z 272.31 [M+H]+ (ES+).

### Step D: Preparation of 4,4-diethoxy-1-((1R,3S)-3-(ethoxymethyl)cyclopentyl)piperidine

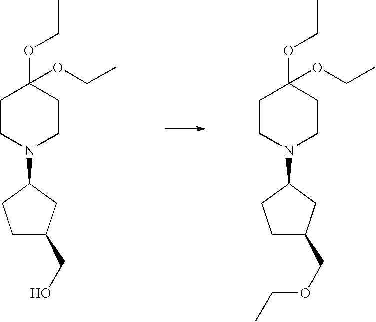

Iodoethane (0.607 mL, 7.52 mmol) was added to a mixture of ((1S,3R)-3-(4,4-diethoxypiperidin-1-yl)cyclopentyl)methanol (0.51 g, 1.88 mmol) and crushed potassium hydroxide (0.422 g, 7.52 mmol) in dimethylsulfoxide (5 mL). The resulting mixture was stirred at room temperature overnight. Brine (15 mL) and diethyl ether (20 mL) were added to the reaction mixture. The layers separated, and the aqueous layer was extracted with additional diethyl ether (2×20 mL). The combined organic extract was washed with brine (15 mL), dried over MgSO₄, and concentrated under reduced pressure to give the title compound (0.5g, 89%), which was used in the subsequent step without further purification. MS m/z 300.35 [M+H]+ (ES+).

### Step E: Preparation of 1-((1R,3S)-3-(ethoxymethyl)cyclopentyl)piperidin-4-one

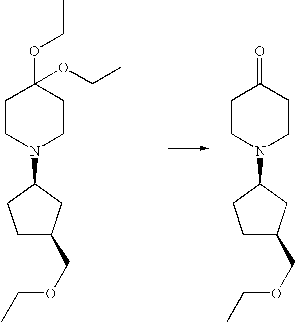

Hydrochloric acid (2N, 15 mL, 30.00 mmol) was added to a solution of 4,4-diethoxy-1-((1R,3S)-3-(ethoxymethyl)cyclopentyl)piperidine (0.5 g, 1.67 mmol) in methanol (15 mL). The resulting mixture was heated at 80° C. for 3 hours. Volatiles were removed under reduced pressure. Saturated solution of sodium bicarbonate was added slowly to the remaining solution until pH >8 and extracted with dichloromethane (2×30 mL). Combined organic extracts was dried over MgSO₄, filtered and concentrated under reduced pressure to give the title compound. The crude product (0.220 g, 58%) was used in the subsequent step without further purification. MS m/z 226.06 [M+H]+ (ES+).

### Step F: Preparation of (1S,2S)-2-(1-((1R,3S)-3-(ethoxymethyl)cyclopentyl)piperidin-4-ylamino)cyclohexanol

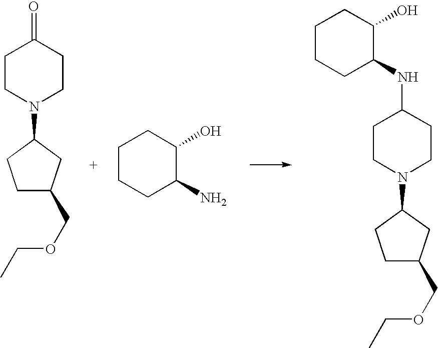

Sodium triacetoxyborohydride (0.310 g, 1.46 mmol) was added to a solution of (1S,2S)-2-aminocyclohexanol (0.112 g, 0.98 mmol) and 1-((1R,3S)-3-(ethoxymethyl)cyclopentyl)piperidin-4-one (0.22 g, 0.98 mmol) in dichloromethane (5 mL), and the resulting mixture was stirred at room temperature overnight. Dichloromethane (10 mL) was added to the reaction mixture and washed with saturated aqueous solution of sodium bicarbonate. The combined organic extract was dried over magnesium sulfate, filtered and concentrated under reduced pressure. The residue was purified by preparative LC/MS (High pH, 40-60% acetonitrile in water) to give the title compound (0.150 g, 47.3%). MS m/z 325.23 [M+H]+ (ES+).

### Step G: Preparation of 2-chloro-N-(1-((1R,3S)-3-(ethoxymethyl)cyclopentyl)piperidin-4-yl)-N-((1S,2S)-2-hydroxycyclohexyl)acetamide

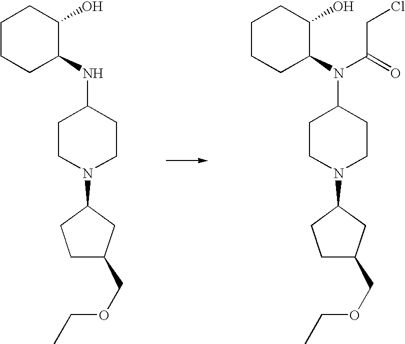

2-chloroacetyl chloride (0.057 g, 0.51 mmol) was added to a solution of (1S,2S)-2-(1-((1R,3S)-3-(ethoxymethyl)cyclopentyl)piperidin-4-ylamino)cyclohexanol (0.15 g, 0.46 mmol) and triethylamine (0.129 mL, 0.92 mmol) in dichloromethane (15 mL), and the resulting mixture was stirred at room temperature for 3 hours. Brine (10 mL) was added to the reaction mixture, and the phases were separated. The organic extract was dried over MgSO₄, filtered and concentrated under reduced pressure. The crude product (0.130 g, 70.1%) was used for the subsequent step without further purification. MS m/z 401.21 [M+H]+ (ES+).

### EXAMPLE 43

### (4aS,8aS)-4-(1-((1S,3R)-3-(ethoxymethyl)cyclopentyl)piperidin-4-yl)hexahydro-2H-benzo[b][1,4]oxazin-3(4H)-one

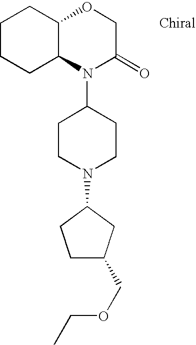

Following an analogous procedure to that described in Example 42 (Step A to Step G), the title compound was made, starting from (1R,3S)-3-aminocyclopentanecarboxylic acid. 1H NMR (400 MHz, CHLOROFORM-D) δ ppm 0.96-1.10 (m, 1 H) 1.13 (t, J=7.03 Hz, 3 H) 1.16-1.31 (m, 2 H) 1.31-1.48 (m, 4 H) 1.51-1.87 (m, 7 H) 1.88-2.05 (m, 3 H) 2.05-2.27 (m, 3 H) 2.31-2.54 (m, 2 H) 2.93-3.10 (m, 2 H) 3.10-3.32 (m, 4 H) 3.41 (q, J=6.77 Hz, 2 H) 3.89 (tt, J=12.30, 3.91 Hz, 1 H) 4.03-4.26 (m, 2 H). Exact mass calculated for C21 H36N2O3 364.27259, found: 365.27987.

### EXAMPLE 44 (DIASTEREOMER 3) AND EXAMPLE 45 (DIASTEREOMER 4)

### Diastereomer 3 and Diastereomer 4 of (4aS,8aS)-4-(1-(3-(ethoxymethyl)cyclopentyl)piperidin-4-yl)hexahydro-2H-benzo[b][1,4]oxazin-3(4H)-one

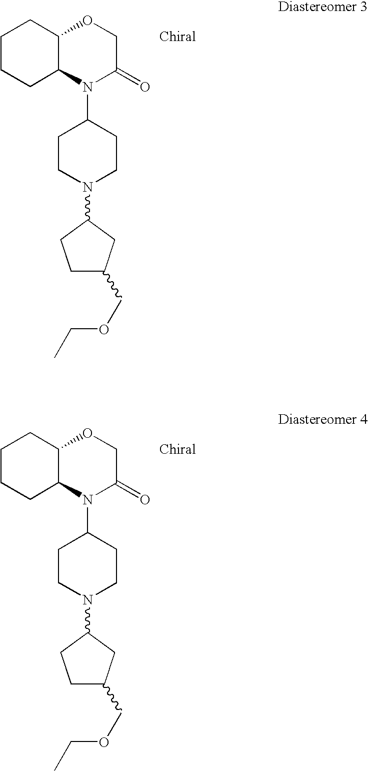

### Step A: Preparation of (4aS,8aS)-4-(1-(3-(ethoxymethyl)cyclopentyl)piperidin-4-yl)hexahydro-2H-benzo[b][1,4]oxazin-3(4H)-one (mixture of diastereomers)

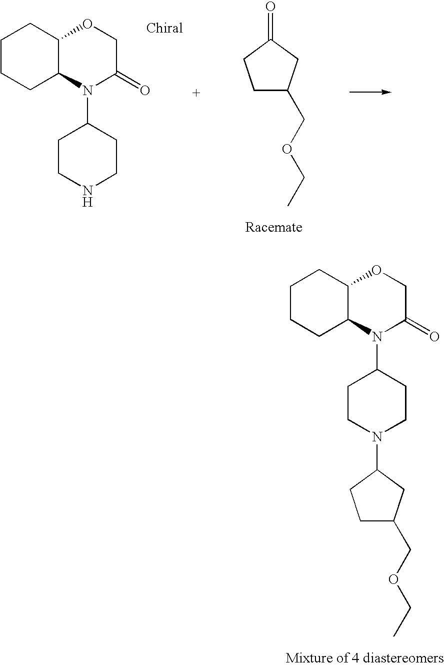

Following an analogous procedure to that described in Example 13 and Example 14, the title compound was made from (4aS,8aS)-4-(piperidin-4-yl)hexahydro-2H-benzo[b][1,4]oxazin-3(4H)-one (HCl salt) (0.319 g, 1.16 mmol), and 3-(ethoxymethyl)cyclopentanone (0.1815 g, 1.28 mmol). The crude product was purified by preparative LC/MS (gradient 30-50% CH₃CN in H₂O) to provide a mixture of diastereomers of the title product (0.305 g, 72.1%).

### Step B: Preparation of Diastereomer 3 and Diastereomer 4 of (4aS,8aS)-4-(1-(3-(ethoxymethyl)cyclopentyl)piperidin-4-yl)hexahydro-2H-benzo[b][1,4]oxazin-3(4H)-one

The diastereomers of (4aS,8aS)-4-(1-(3-(ethoxymethyl)cyclopentyl)piperidin-4-yl)hexahydro-2H-benzo[b][1,4]oxazin-3(4H)-one (0.288 g, 0.79 mmol) were separated by chiral phase HPLC (ChiralPak AD column, 20% (EtOH +0.1% diethylamine):80% heptane) followed by a another chiral phase HPLC (ChiralPak AD column, 10% (EtOH+0.1% diethylamine):10% (MeOH+0.1% diethylamine):80% heptane if necessary).

The first eluting isomer (0.040 g, 13.82%), under the first HPLC conditions (ChiralPak AD column, 20% (EtOH+0.1% diethylamine):80% heptane) is Diastereomer 1 of the title compound and is identical to Example 43.

The second eluting isomer (6.60 mg, 2.292%), under the first HPLC conditions (ChiralPak AD column, 20% (EtOH+0.1% diethylamine):80% heptane) is Diastereomer 2 of the title compound and is identical to Example 42.

The third eluting isomer (4.70 mg, 1.632%), under the first HPLC conditions (ChiralPak AD column, 20% (EtOH +0.1% diethylamine):80% heptane) is Diastereomer 3 of the title compound (Example 44). 1H NMR (400 MHz, CHLOROFORM-D) δ ppm 1.12-1.51 (m, 9 H, including a triplet at 1.19),1.58-2.38 (m, 14 H), 2.41-2.63 (m, 2 H), 3.00-3.35 (m, 6 H), 3.47 (q, J=7.0 Hz, 2 H), 3.93-4.09 (m, 1 H), 4.13-4.30 (m, 2 H). Exact mass calculated for C21 H36N2O3+H: 365.2799. Found: 365.2801.

The fourth eluting isomer (0.012 g, 4.27%), under the first HPLC conditions (ChiralPak AD column, 20% (EtOH+0.1% diethylamine):80% heptane) is Diastereomer 4 of the title compound (Example 45). 1H NMR (400 MHz, CHLOROFORM-D) δ ppm 1.07-1.54 (m, 9 H, including a triplet at 1.19),1.57-2.39 (m, 14 H), 2.41-2.65 (m, 2 H), 2.98-3.35 (m, 6 H), 3.46 (q, J=7.0 Hz, 2 H), 4.09 (m, 1 H), 4.13-4.31 (m, 2 H).

### EXAMPLE 46

### (4aR,8aR)-4-(1-((1R,3S)-3-(ethoxymethyl)cyclopentyl)piperidin-4-yl)hexahydro-2H-benzo[b][1,4]oxazin-3(4H)-one

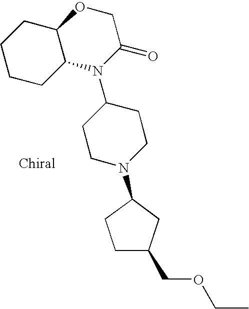

### Step A: Preparation of (4aR,8aR)-4-(1-((1R,3S)-3-(ethoxymethyl)cyclopentyl)piperidin-4-yl)hexahydro-2H-benzo[b][1,4]oxazin-3(4H)-one

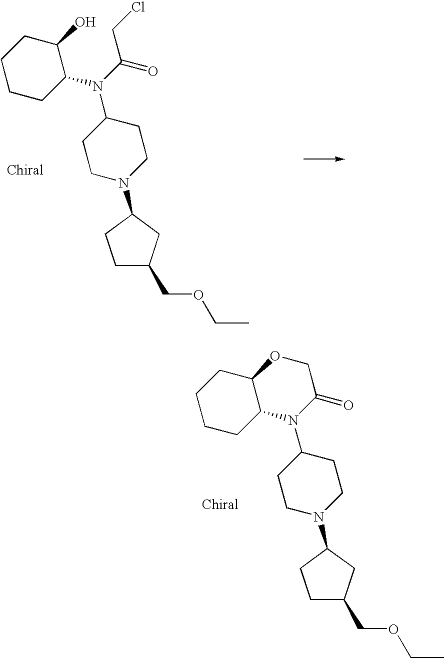

Following an analogous procedure to that described in Step A of Example 42, the title compound was made from 2-chloro-N-(1-((1R,3S)-3-(ethoxymethyl)cyclopentyl)piperidin-4-yl)-N-((1R,2R)-2-hydroxycyclohexyl)acetamide (0.125 g, 0.31 mmol). The crude product was purified by preparative LC/MS (high pH, 40-60% MeCN) to give the title product (0.060 g, 48.0%).1H NMR (400 MHz, CHLOROFORM-D) δ ppm 0.95-1.08 (m, 1 H) 1.08-1.14 (m, 3 H) 1.14-1.48 (m, 5 H) 1.52-1.85 (m, 6 H) 1.86-2.05 (m, 5 H) 2.04-2.28 (m, 3 H) 2.31-2.52 (m, 2 H) 3.02 (dd, J=19.92, 10.94 Hz, 2 H) 3.09-3.31 (m, 4 H) 3.40 (q, J=6.38 Hz, 2 H) 3.79-3.95 (m, 1 H) 4.03-4.23 (m, 2 H). MS m/z 365.2 [M+H]+ (ES+).

### Step B: Preparation of (1R,2R)-2-(1-((1R,3S)-3-(ethoxymethyl)cyclopentyl)piperidin-4-ylamino)cyclohexanol

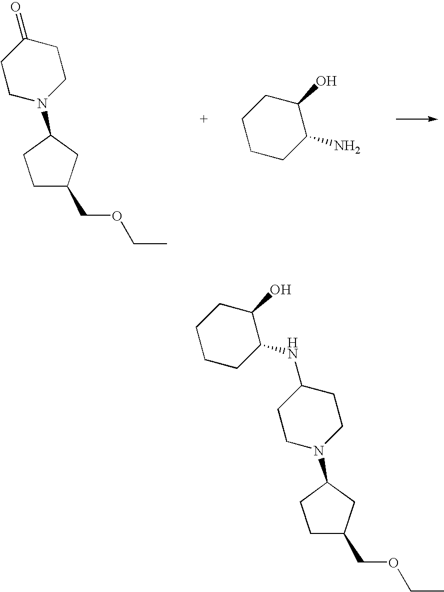

Following an analogous procedure to that described in Step F of Example 42, the title compound was made from (1R,2R)-2-aminocyclohexanol (0.082 g, 0.71 mmol) and 1-((1R,3S)-3-(ethoxymethyl)cyclopentyl)piperidin-4-one (0.16 g, 0.71 mmol). The crude product (0.11 g, 47%) was used in the subsequent step without further purification. MS m/z 325.4 [M+H]+ (ES+).

### Step C: Preparation of 2-chloro-N-(1-((1R,3S)-3-(ethoxymethyl)cyclopentyl)piperidin-4-yl)-N-((1R,2R)-2-hydroxycyclohexyl)acetamide

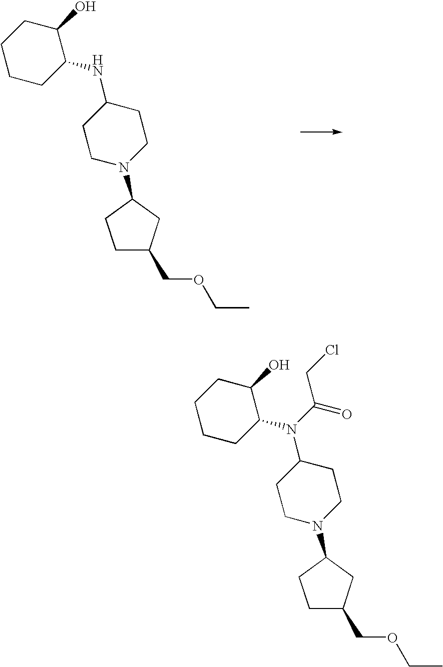

Following an analogous procedure to that described in Step G of Example 42, the title compound was made from (1R,2R)-2-(1-((1R,3S)-3-(ethoxymethyl)cyclopentyl)piperidin-4-ylamino)cyclohexanol. The crude product was (0.125g, 92%) was used for the subsequent step without further purification. MS m/z 401.36 [M+H]+(ES+).

### EXAMPLE 47

### (4aS,7aR)-4-(1-((1s,4R)-4-((cyclopropylmethoxy)methyl)cyclohexyl)piperidin-4-yl)hexahydrocyclopenta[b][1,4]oxazin-3(2H)-one

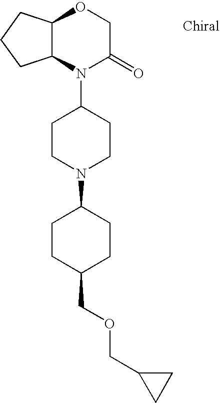

### Step A: Preparation of (4aS,7aR)-4-(1-((1s,4R)-4-((cyclopropylmethoxy)methyl)cyclohexyl)piperidin-4-yl)hexahydrocyclopenta[b][1,4]oxazin-3(2H)-one

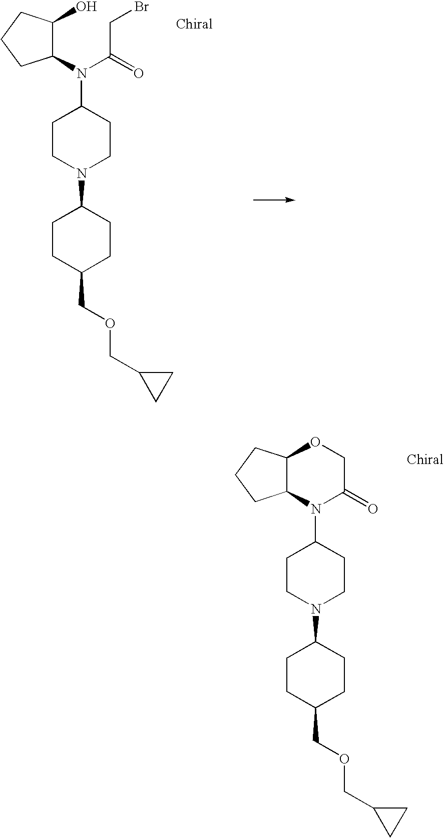

Following an analogous procedure to that described in Step A of Example 32, the title compound was made from 2-bromo-N-(1-((1s,4R)-4-((cyclopropylmethoxy)methyl)cyclohexyl)piperidin-4-yl)-N-((1S,2R)-2-hydroxycyclopentyl)acetamide (60 mg, 0.13 mmol). The crude product was purified by preparative LC/MS (high pH, 40-60% acetonitrile in water) to give the title product.

The oxalate salt was made by addition of an oxalic acid solution in water. 1H NMR (400 MHz, CHLOROFORM-D) δ ppm 0.16-0.25 (m, 2 H) 0.45-0.60 (m, 2 H) 0.99-1.13 (m, 1 H) 1.41-1.92 (m, 18 H) 1.91-2.06 (m, 1 H) 2.06-2.32 (m, 3 H) 2.98-3.10 (m, 2 H) 3.26 (d, J=7.03 Hz, 2 H) 3.39 (d, J=7.42 Hz, 2 H) 3.41-3.48 (m, 1 H) 3.99 (t, J=3.91 Hz, 1 H) 4.09-4.15 (m, J=16.41 Hz, 1 H) 4.19-4.27 (m, J=16.80 HJz, 1 H) 4.28-4.38 (m, 1 H); MS m/z 391.2 [M+H]+(ES+). Exact mass calculated for C23H38N2O3 391.29552 [M+H]+, found 391.29515.

### Step B: Preparation of (1R,2S)-2-(1-((1s,4R)-4-((cyclopropylmethoxy)methyl)cyclohexyl)piperidin-4-yla mino)cyclopentanol

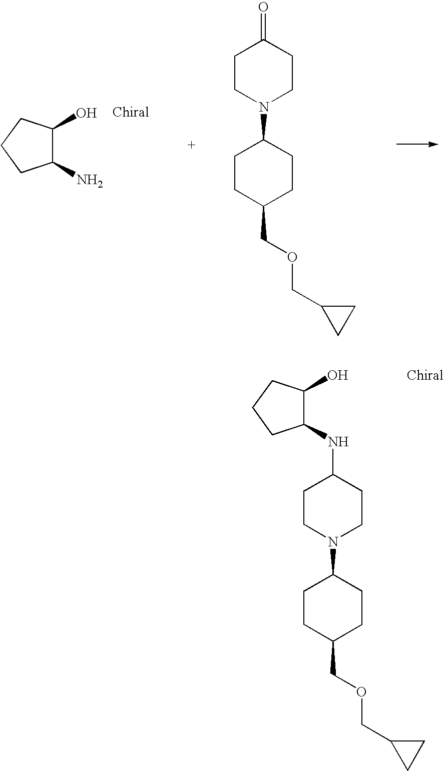

Triethylamine (0.156 ml, 1.12 mmol) was added to a solution of (1R,2S)-2-aminocyclopentanol (63.0 mg, 0.62 mmol) and 1-((1s,4s)-4-((cyclopropylmethoxy)methyl)cyclohexyl)piperidin-4-one (188 mg, 0.62 mmol) in dichloromethane (3.996 ml) under a nitrogen atmosphere and stirred at room temperature for 3 hours. Sodium triacetoxyborohydride (198 mg, 0.93 mmol) was added and the reaction was stirred at room temperature overnight. Solid NaHCO₃ (15 mg) was added to the reaction mixture. The mixture was stirred at room temperature for 10 minutes and concentrated under reduced pressure. The residue was purified by LC/MS (high pH, 40-60% acetonitrile in water) to give the title product (20 mg, 9%). 1H NMR (400 MHz, CHLOROFORM-D) δ ppm 0.17-0.23 (m, 2 H) 0.49-0.57 (m, 2 H) 1.01-1.11 (m, 1 H) 1.29-1.98 (m, 21 H) 2.04-2.14 (m, 2 H) 2.23 (br. s.,1 H) 2.39-2.49 (m,1 H) 2.95 (d, J=9.77 Hz, 2 H) 3.02-3.09 (m, 1 H) 3.26 (d, J=7.03 Hz, 2 H) 3.38 (d, J=7.42 Hz, 2 H) 3.84-3.89 (m,1 H); MS m/z 351.27 [M+H]+ (ES+).

### Step C: Preparation of 2-bromo-N-(1-((1s,4R)-4-((cyclopropylmethoxy)methyl)cyclohexyl)piperidin-4-yl)-N-((1S,2R)-2-hydroxycyclopentyl)acetamide

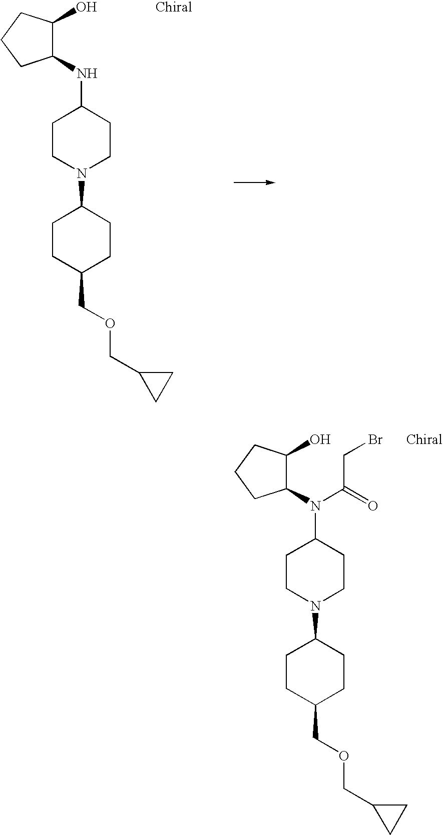

Following an analogous procedure to that described in Step E of Example 32, the title compound was made from (1R,2S)-2-(1-((1s,4R)-4-((cyclopropylmethoxy)methyl)cyclohexyl)piperidin-4-ylamino)cyclopentanol. The crude product was used in the subsequent step without further purification. MS m/z 473.33 [M+H]+ (ES+).

### EXAMPLE 48 (ENANTIOMER 1) AND EXAMPLE 49 (ENANTIOMER 2)

### Enantiomer 1 and Enantiomer 2 of 4-(1-((1s,4R)-4-((cyclopropylmethoxy)methyl)cyclohexyl)piperidin-4-yl)hexahydrocyclopenta[b][1,4]oxazin-3(2H)-one

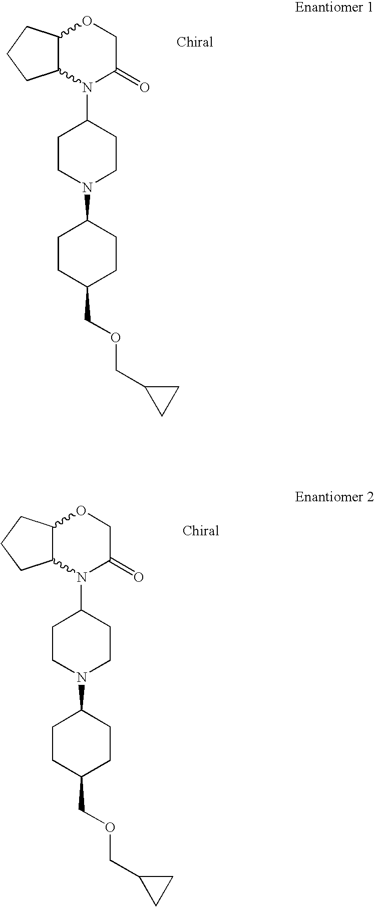

### Step A: Preparation of Enantiomer 1 and Enantiomer 2 of 4-(1-((1s,4R)-4-((cyclopropylmethoxy)methyl)cyclohexyl)piperidin-4-yl)hexahydrocyclopenta[b][1,4]oxazin-3(2H)-one

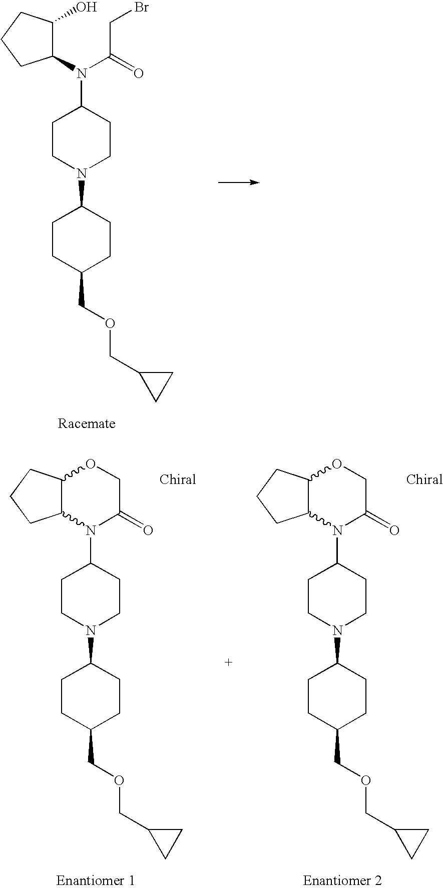

Following an analogous procedure to that described in Step A of Example 32, the title compound was made from 2-bromo-N-(1-((1s,4R)-4-((cyclopropylmethoxy)methyl)cyclohexyl)piperidin-4-yl)-N-(trans-2-hydroxycyclopentyl)acetamide (702 mg, 1.49 mmol). The crude product was purified by preparative LC/MS (high pH, 40-60% acetonitrile in water) to give a mixture of enantiomers of the title compound (42 mg, 7.22%).

Enantiomer 1 and Enantiomer 2 of the title compound were separated by chiral SFC (ChiralPak OD-H column, 25:75 (Isopropanol containing 0.1% dimethylethylamine): supercritical CO₂).

First eluting fraction is Enantiomer 1 of the title compound (Example 48) (15.0 mg, 2.4%), Retention time: 5.02 minutes (ChiralPak OD-H column, 25:75 (Isopropanol containing 0.1% dimethylethylamine): supercritical CO₂). 1H NMR (400 MHz, CHLOROFORM-D) δ ppm 0.17-0.23 (m, 2 H), 0.50-0.57 (m, 2 H), 1.01-1.12 (m, 1 H), 1.40-1.62 (m, 8 H), 1.62-1.72 (m, 4 H), 1.73-1.90 (m, 4 H), 1.96 (quint d J=11.91, 4.30 Hz, 2 H), 2.17 (td, J=1 1.62, 2.15 Hz, 2 H), 2.21-2.31 (m, 2 H), 2.93-3.07 (m, J=24.22, 10.94, 2.73 Hz, 2 H), 3.26 (d, J=6.64 Hz, 2 H), 3.37 (d, J=7.42 Hz, 2 H), 3.39-3.46 (m,1 H), 3.73 (ddd, 1 H), 4.23-4.31 (m,1 H), 4.33 (d, J=4.69 Hz, 2 H). Exact mass calculated for C23H38N2O391.29552 [M+H]+, found 391.29544.

Second eluting fraction is Enantiomer 2 of the title compound (Example 49). Retention time: 5.44 minutes (ChiralPak OD-H column, 25:75 (Isopropanol containing 0.1% dimethylethylamine): supercritical C0₂). 1H NMR (400 MHz, CHLOROFORM-D) δ ppm 0.15-0.24 (m, 2 H), 0.47-0.58 (m, 2 H), 0.99-1.12 (m,1 H), 1.40-1.62 (m, 8 H), 1.62-1.72 (m, 4 H), 1.72-1.90 (m, 4 H), 1.90-2.05 (m, 2 H), 2.17 (td, J=11.52, 2.34 Hz, 2 H), 2.22-2.31 (m, 2 H), 2.93-3.08 (m, 2 H), 3.26 (d, J=7.03 Hz, 2 H), 3.37 (d, J=7.42 Hz, 2 H), 3.39-3.46 (m, 1 H), 3.73 (ddd, J=10.94, 9.18, 7.23 Hz, 1 H), 4.23-4.31 (m, 1 H), 4.33 (d, J=5.08 Hz, 2 H). Exact mass calculated for C23H38N2O3 391.29552 [M+H]+, found 391.29579.

### Step B: Preparation of 2-(1-((1s,4R)-4-((cyclopropylmethoxy)methyl)cyclohexyl)piperidin-4-ylamino)cyclopentanol

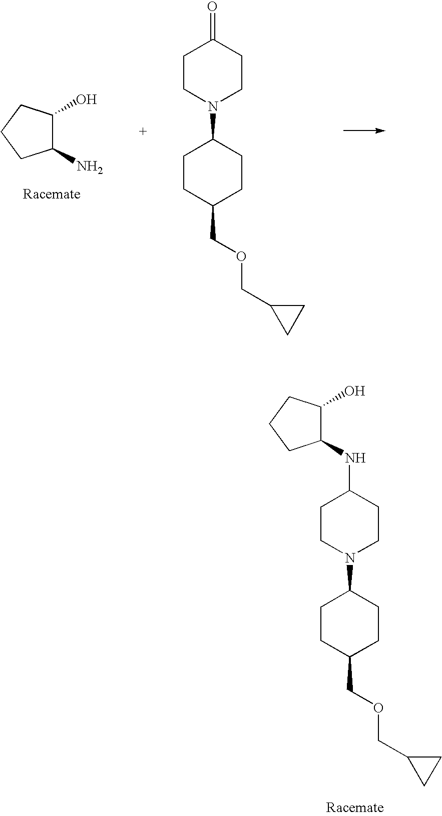

Following an analogous procedure to that described in Step B of Example 47, the title compound was made from trans-2-aminocyclopentanol (369 mg, 2.68 mmol) and 1-((1s,4s)-4-((cyclopropylmethoxy)methyl)cyclohexyl)piperidin-4-one (810 mg, 2.68 mmol). The crude product was purified by preparative LC/MS (high pH, 40-60% acetonitrile in water) to give the title (521 mg, 55.4%). MS m/z 351.37 [M+H]+ (ES+).

### Step C: Preparation of 2-bromo-N-(1-((1s,4R)-4-((cyclopropylmethoxy)methyl)cyclohexyl)piperidin-4-yl)-N-(2-hydroxycyclopentyl)acetamide

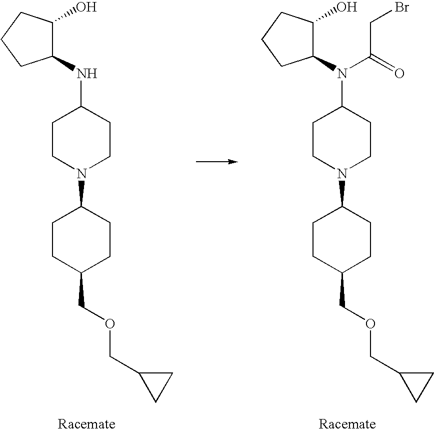

Following an analogous procedure to that described in Step E of Example 32, the title compound was made from trans-2-(1-((1s,4R)-4-((cyclopropylmethoxy)methyl)cyclohexyl)piperidin-4-ylamino)cyclopentanol (521 mg, 1.49 mmol). The crude product was used in the subsequent step without further purification. MS m/z 473.31 [M+H]+ (ES+).

### EXAMPLE 50 (DIASTEREOMER 1) AND EXAMPLE 51 (DIASTEREOMER 2)

### Diastereomer 1 and diastereomer 2 of (4aR,8aS)-1-(1-(4-((2,2-difluoroethoxy)methyl)cyclohexyl)piperidin-4-yl)octahydroquinazolin-2(1 H)-one

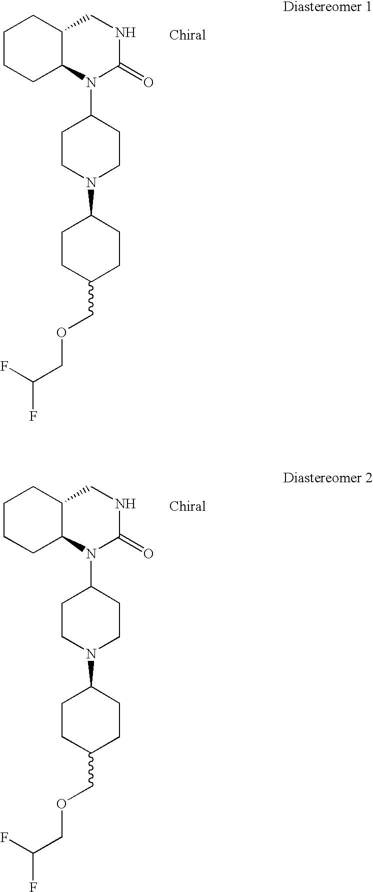

### Step A: Preparation of Diastereomer 1 and diastereomer 2 of (4aR,8aS)-1-(1-(4-((2,2-difluoroethoxy)methyl)cyclohexyl)piperidin-4-yl)octahydroquinazolin-2(1 H)-one

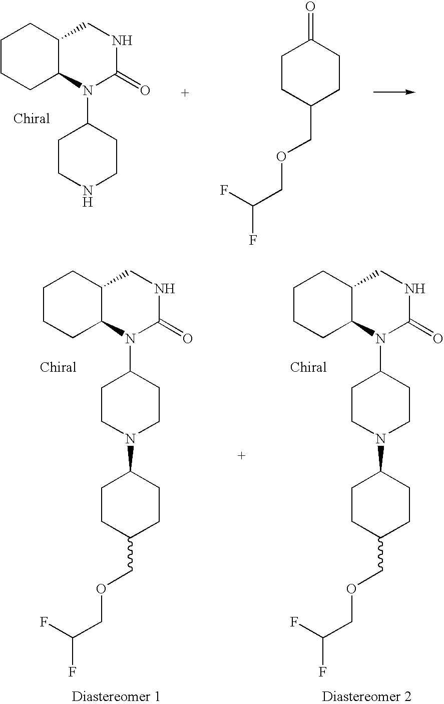

Following an analogous procedure to that described in Example 13 and Example 14, the title compound was made from (4aR,8aS)-1-(piperidin-4-yl)octahydroquinazolin-2(1 H)-one (HCl salt) (0.2517 g, 0.92 mmol) and 4-((2,2-difluoroethoxy)methyl)cyclohexanone (0.194 g, 1.01 mmol). The crude product was purified by preparative LC/MS (high pH, 50-70% acetonitrile in water) followed by SFC on a chiral stationary phase (ChiralPak AD column, 55% (EtOH+0.1% DMEA):CO₂) to give Diastereomer 1 and Diastereomer 2 of the title compound.

The first eluting isomer (0.049 g, 12.97%) is Diastereomer 1 of the title compound (Example 50). Retention time: 2.67 minutes (ChiralPak AD-H column, 55% EtOH with 0.1% DMEA, supercritical C0₂). 1H NMR (400 MHz, CHLOROFORM-D) δ ppm 0.96-1.38 (m, 5 H), 1.40-1.95 (m, 12 H), 2.02-2.49 (m, 7 H), 2.83-3.14 (m, 6 H), 3.47 (d, J=7.4 Hz, 2 H), 3.58-3.79 (m, 3 H), 4.65 (d, J=4.7 Hz, 1 H), 5.86 (tt, J=55.6, 4.3, 4.1 Hz, 1 H). Exact mass calculated for C22H37F2N3O2 414.2927 [M+H]+, found 414.2933.

The second eluting isomer (0.022 g, 5.68%) is Diastereomer 2 of the title compound (Example 51). Retention time: 3.39 minutes (ChiralPak AD-H column, 55% EtOH with 0.1% DMEA, supercritical C0₂). 1H NMR (400 MHz, CHLOROFORM-D) δ ppm 0.87-1.39 (m, 9 H), 1.43-1.96 (m, 10 H), 2.10-2.36 (m, 5 H), 2.38-2.51 (m,1 H) -3.06 (m, 5 H), 3.33 (d, J=6.2 Hz, 2 H), 3.63 (td, J=14.1, 4.3 Hz, 2 H), 3.73-3.88 (m, 1 H), 4.61 (d, J=4.7 Hz, 1 H), 5.86 (tt, J=55.5, 4.1 Hz, 1 H). Exact mass calculated for C22H37F2N3O2 414.2927 [M+H]+, found 414.2929.

1. A compound of formula I, a pharmaceutically acceptable salt thereof, diastereomer, enantiomer, or mixture thereof: 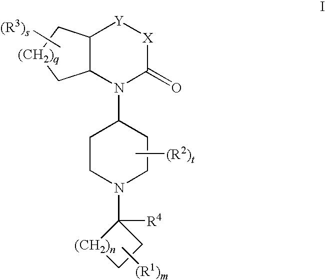
wherein
each R¹ is independently selected from fluoro, C₃₋₇cycloalkyl, C₁₋₇alkyl, C₂₋₆alkenyl, C₂₋₆alkynyl, C₁₋₇alkoxy, C₃₋₇cycloalkoxy-C₁₋₆alkyl, C₁₋₆alkoxy-C₁₋₆alkyl, C₂₋₆alkenyloxy-C₁₋₆alkyl, C₂₋₆alkynyloxy, C₂₋₆alkynyloxy-C₁₋₆alkyl, C₁₋₆alkylamino, di-C₁₋₆alkylamino, C₃₋₇heterocycloalkyloxy, C₃₋₇heterocycloalkyl, C₆₋₁₀aryl-C₁₋₃alkoxy, C₆₋₁₀aryl-C₁₋₃alkyl, C₃₋₉heteroaryl-C₁₋₃alkoxy, C₃₋₉heteroaryl-C₁₋₃alkyl, C₃₋₇heterocycloalkyl-C₁₋₃alkoxy, C₃₋₇heterocycloalkyl-C₁₋₃alkyl, C₃₋₇cycloalkyloxy, C₃₋₇cycloalkyl-C₁₋₃alkyl, C₃₋₇cycloalkyl-C₁₋₃alkoxy and C₃₋₇cycloalkyl-C₁₋₃alkoxy-C₁₋₃alkyl, wherein said C₃₋₇cycloalkyl, C₁₋₇alkyl, C₂₋₆alkenyl, C₂₋₆alkynyl, C₁₋₇alkoxy, C₃₋₇cycloalkoxy-C₁₋₆alkyl, C₁₋₆alkoxy-C₁₋₆alkyl, C₂₋₆alkenyloxy, C₂₋₆alkenyloxy-C₁₋₆alkyl, C₂₋₆alkynyloxy, C₂₋₆alkynyloxy-C₁₋₆alkyl, C₁₋₆alkylamino, di-C₁₋₆alkylamino, C₃₋₇heterocycloalkyloxy, C₃₋₇heterocycloalkyl, C₆₋₁₀aryl-C₁₋₃alkoxy, C₆₋₁₀aryl-C₁₋₃alkyl, C₃₋₉heteroaryl-C₁₋₃alkoxy, C₃₋₉heteroaryl-C₁₋₃alkyl, C₃₋₇heterocycloalkyl-C₁₋₃alkoxy, C₃₋₇heterocycloalkyl-C₁₋₃alkyl, C₃₋₇cycloalkyloxy, C₃₋₇cycloalkyl-C₁₋₃alkyl, C₃₋₇cycloalkyl-C₁₋₃alkoxy and C₃₋₇cycloalkyl-C₁₋₃alkoxy-C₁₋₃alkyl are optionally substituted with one or more group selected from phenyl, C₃₋₆cycloalkyl, C₂₋₅heterocycloalkyl, C₃₋₅heteroaryl, —CN, —SR, —OR, —O(CH₂)ₚ—OR, R, —C(═O)—R, —CO₂R, —SO₂R, —SO₂NRR′, halogen, —NO₂, —NRR′, —(CH₂)ₚNRR′, and —C(═O)—NRR;
each R² is independently selected from halogen, C₁₋₆alkyl, C₃₋₇cycloalkyl, halogenated C₁₋₆alkyl, C₁₋₆alkoxy, and halogenated C₁₋₆alkoxy;
each R³ is independently selected from halogen, C₁₋₆alkyl, C₃₋₇cycloalkyl, halogenated C₁₋₆alkyl, CN, C₁₋₆alkoxy, and halogenated C₁₋₆alkoxy; or two R³ together form a C₁₋₆alkylene, C₁₋₆alkylenoxy, or halogenated C₁₋₆alkylene;
R⁴ is hydrogen, C₁₋₆ alkyl, or C₁₋₆ haloalkyl;
q is 1, 2, 3 or 4;
p is 2, 3 or 4; s is 0, 1, 2, 3, or 4; t is 0, 1, 2, 3, or 4; n is 0, 1, 2, 3 or 4; m is 0, 1, 2, 3 or 4;
Y is —CR⁵R⁶—, —O—, or —S—;
X is —CR⁵R⁶—, —NR⁷—, —O—, or —S—;
each R⁵, R⁶ and R⁷ are independently selected from hydrogen, C₁₋₆alkyl, C₂₋₆alkenyl and halogenated C₁₋₆alkyl; and
each R and R′ are independently C₁₋₆alkyl, C₂₋₆alkenyl or halogenated C₁₋₆alkyl, with a proviso that at least one of X and Y is —CR⁵R⁶—, with a further proviso that the compound is not (4aS,8aS)-4-(1-(4-(ethoxymethyl)-1-methylcyclohexyl)piperidin-4-yl)hexahydro-2H-benzo[b][1,4]oxazin-3(4H)-one.

2. A compound as claimed in claim 1, wherein X is —CH₂— or —NH—.

3. A compound as claimed in claim 1, Y is CH₂ or O.

4. A compound as claimed in claim 1, wherein R′ is selected from C₁₋₆alkoxy, C₁₋₆alkoxy-C₁₋₆alkyl, halogenated C₁₋₆alkoxy-C₁₋₆alkyl, C₁₋₆alkyl, C₃₋₆alkenyloxy, C₃₋₆alkynyloxy, C₃₋₆cycloalkyl, C₃₋₆cycloalkyl-C₁₋₃alkoxy, halogenated C₁₋₆alkyl, halogenated C₃₋₆cycloalkyl-C₁₋₃alkoxy, or halogenated C₃₋₆cycloalkyl.

5. A compound as claimed in claim 1, wherein R⁴ is hydrogen.

6. A compound as claimed in claim 1, wherein each R² is independently selected from methyl, fluoromethyl, difluoromethyl, trifluoromethyl, ethyl, C₁₋₃alkoxy and fluoro.

7. A compound as claimed in claim 1, wherein each R³ is independently selected from methyl, fluoromethyl, difluoromethyl, trifluoromethyl, ethyl, C₁₋₃alkoxy and fluoro.

8-14. (canceled)

15. A compound selected from
(4aR,8aS)-1-(1-(4-(propoxymethyl)cyclohexyl)piperidin-4-yl)octahydroquinazolin-2(H)-one;
(4aR,8aS)-1-(1-(4-(isopropoxymethyl)cyclohexyl)piperidin-4-yl)octahydroquinazolin-2(1H)-one;
(4aR,8aS)-1-(1-(4-propoxycyclohexyl)piperidin-4-yl)octahydroquinazolin-2(1 H)-one;
(4aR,8aS)-1-(1-(4-isopropoxycyclohexyl)piperidin-4-yl)octahydroquinazolin-2(1 H)-one;
(4aR,8aS)-1-(1-(4-(ethoxymethyl)cyclohexyl)piperidin-4-yl)octahydroquinazolin-2(1 H)-one;
(4aR,8aS)-1-(1-(4-(prop-2-ynyloxy)cyclohexyl)piperidin-4-yl)octahydroquinazolin-2(1H)-one;
(4aR,8aS)-1-(1-cyclopentylpiperidin-4-yl)octahydroquinazolin-2(1 H)-one;
(4aR,8aS)-1-(1-(4-ethylcyclohexyl)piperidin-4-yl)octahydroquinazolin-2(1H)-one;
(4aR,8aS)-1-(1-cyclohexylpiperidin-4-yl)octahydroquinazolin-2(1 H)-one;
(4aS ,8aS)-4-(1-(4-(ethoxymethyl)cyclohexyl)piperidin-4-yl)hexahydro-2H-benzo[b][1,4]oxazin-3(4H)-one;
(4aS,8aS)-4-(1-(4-(isopropoxymethyl)cyclohexyl)piperidin-4-yl)hexahydro-2H-benzo[b][1,4]oxazin-3(4H)-one;
(4aS,8aS)-4-(1-(4-propoxycyclohexyl)piperidin-4-yl)hexahydro-2H-benzo[b][1,4]oxazin-3(4H)-one;
(4aS ,8aS)-4-(1-(4-(isopropoxymethyl)cyclohexyl)piperidin-4-yl)hexahydro-2H-benzo[b][1,4]oxazin-3(4H)-one;
(4aS,8aS)-4-(1-(4-(cyclopropylmethoxy)cyclohexyl)piperidin-4-yl)hexahydro-2H-benzo[b][1,4]oxazin-3(4H)-one;
(4aS ,8aS)-4-(1-(4-((cyclopropylmethoxy)methyl)cyclohexyl)piperidin-4-yl)hexahydro-2H-benzo[b][1,4]oxazin-3(4H)-one;
(4aS ,8aS)-4-(1-(4-((2-fluoroethoxy)methyl)cyclohexyl)piperidin-4-yl)hexahydro-2H-benzo[b][1,4]oxazin-3(4H)-one;
(4aS ,8aS)-4-(1-(4-((2,2-difluoroethoxy)methyl)cyclohexyl)piperidin-4-yl)hexahydro-2H-benzo[b][1,4]oxazin-3(4H)-one;
(4aS,8aS)-4-(1-(4-((cyclobutylmethoxy)methyl)cyclohexyl)piperidin-4-yl)hexahydro-2H-benzo[b][1,4]oxazin-3(4H)-one;
(4aS ,8aS)-4-(1-(4-(ethoxymethyl)-4-methylcyclohexyl)piperidin-4-yl)hexahydro-2H-benzo[b][1,4]oxazin-3(4H)-one;
(4aR,8aR)-4-(1-((1s,4S)-4-((cyclopropylmethoxy)methyl)cyclohexyl)piperidin-4-yl)hexahydro-2H-benzo[b][1,4]oxazin-3(4H)-one;
(cis)-4-(1-((1s,4S)-4-(ethoxymethyl)cyclohexyl)piperidin-4-yl)hexahydro-2H-benzo[b][1,4]oxazin-3(4H)-one;
(4aR,8aR)-6,6-difluoro-4-(1-(4-(isopropoxymethyl)cyclohexyl)piperidin-4-yl)hexahydro-2H-benzo[b][1,4]oxazin-3(4H)-one;
(4aS,8aS)-6,6-difluoro-4-(1-(4-(isopropoxymethyl)cyclohexyl)piperidin-4-yl)hexahydro-2H-benzo[b][1,4]oxazin-3(4H)-one;
(4aS ,8aS)-4-(1-(3-(ethoxymethyl)cyclobutyl)piperidin-4-yl)hexahydro-2H-benzo[b][1,4]oxazin-3(4H)-one;
(4aS,8aS)-4-(1-(3-((cyclobutylmethoxy)methyl)cyclobutyl)piperidin-4-yl)hexahydro-2H-benzo[b][1,4]oxazin-3(4H)-one;
(4aS ,8aS)-4-(1-(3-((cyclopropylmethoxy)methyl)cyclobutyl)piperidin-4-yl)hexahydro-2H-benzo[b][1,4]oxazin-3(4H)-one;
(4aS,8aS)-4-(1-((1R,3S)-3-(ethoxymethyl)cyclopentyl)piperidin-4-yl)hexahydro-2H-benzo[b][1,4]oxazin-3(4H)-one;
(4aS ,8aS)-4-(1-((S ,3R)-3-(ethoxymethyl)cyclopentyl)piperidin-4-yl)hexahydro-2H-benzo[b][1,4]oxazin-3(4H)-one;
(4aS ,8aS)-4-(1-(3-(ethoxymethyl)cyclopentyl)piperidin-4-yl)hexahydro-2H-benzo[b][1,4]oxazin-3(4H)-one;
(4aR,8aR)-4-(1-((1R,3S)-3-(ethoxymethyl)cyclopentyl)piperidin-4-yl)hexahydro-2H-benzo[b][1,4]oxazin-3(4H)-one;
(4aS,7aR)-4-(1-((1s,4R)-4-((cyclopropylmethoxy)methyl)cyclohexyl)piperidin-4-yl)hexahydrocyclopenta[b][1,4]oxazin-3(2H)-one;
4-(1-((1s,4R)-4-((cyclopropylmethoxy)methyl)cyclohexyl)piperidin-4-yl)hexahydrocyclopenta[b][1,4]oxazin-3(2H)-one;
(4aR,8aS)-1-(1-(4-((2,2-difluoroethoxy)methyl)cyclohexyl)piperidin-4-yl)octahydroquinazolin-2(1 H)-one;
enantiomers thereof, diastereomers thereof, pharmaceutically acceptable salts thereof, and mixtures thereof.

16. (4aS,8aS)-6,6-difluoro-4-(1-(4-(isopropoxymethyl)cyclohexyl)piperidin-4-yl)hexahydro-2H-benzo[b][1,4]oxazin-3(4H)-one, a diastereomer thereof, a pharmaceutically acceptable salt thereof, or a mixture thereof

17. (4aS,8aS)-6,6-difluoro-4-(1-((1R,4S)-4-(isopropoxymethyl)cyclohexyl)piperidin-4-yl)hexahydro-2H-benzo[b][1,4]oxazin-3(4H)-one, a pharmaceutically acceptable salt thereof, or a mixture thereof.

18. (4aS,8aS)-6,6-difluoro-4-(1-((14S,4S)-4-(isopropoxymethyl)cyclohexyl)piperidin-4-yl)hexahydro-2H-benzo[b][1,4]oxazin-3(4H)-one, a pharmaceutically acceptable salt thereof, or a mixture thereof.

19. Diastereomer 2 of (4aS,8aS)-6,6-difluoro-4-(l -(4-(isopropoxymethyl)cyclohexyl)piperidin-4-yl)hexahydro-2H-benzo[b][1,4]oxazin-3(4H)-one as prepared in Example 38, a pharmaceutically acceptable salt thereof, or a mixture thereof.

20. Diastereomer 1 of (4aS,8aS)-6,6-difluoro-4-(1-(4-(isopropoxymethyl)cyclohexyl)piperidin-4-yl)hexahydro-2H-benzo[b][1,4]oxazin-3(4H)-one as prepared in Example 38, a pharmaceutically acceptable salt thereof, or a mixture thereof.

21-24. (canceled)

25. A pharmaceutical composition comprising a compound according to claim 1 and a pharmaceutically acceptable carrier.

26. A method for the therapy of pain in a warm-blooded animal, comprising the step of administering to said animal in need of such therapy a therapeutically effective amount of a compound according to claim 1.

27-30. (canceled)

31. A process for preparing a compound of Formula I, comprising: 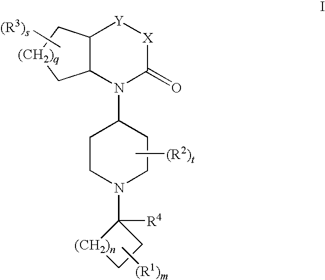
reacting a compound of Formula II with a compound of 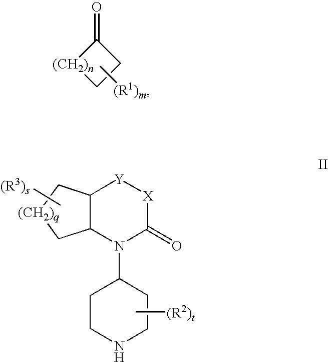
wherein
each R′ is independently selected from fluoro, C₃₋₇cycloalkyl, C₁₋₇alkyl, C₂₋₆alkenyl, C₂₋₆alkynyl, C₁₋₇alkoxy, C₃₋₇cycloalkoxy-C₁₋₆alkyl, C₁₋₆alkoxy-C₁₋₆alkyl, C₂₋₆alkenyloxy, C₂₋₆alkenyloxy-C₁₋₆alkyl, C₂₋₆alkynyloxy, C₂₋₆alkynyloxy-C₁₋₆alkyl, C₁₋₆alkylamino, di-C₁₋₆alkylamino, C₃₋₇heterocycloalkyloxy, C₃₋₇heterocycloalkyl, C₆₋₁₀aryl-C₁₋₃alkoxy, C₆₋₁₀aryl-C₁₋₃alkyl, C₃₋₉heteroaryl-C₁₋₃alkoxy, C₃₋₉heteroaryl-C₁₋₃alkyl, C₃₋₇heterocycloalkyl-C₁₋₃alkoxy, C₃₋₇heterocycloalkyl-C₁₋₃alkyl, C₃₋₇cycloalkyloxy, C₃₋₇cycloalkyl-C₁₋₃alkyl, C₃₋₇cycloalkyl-C₁₋₃alkoxy and C₃₋₇cycloalkyl-C₁₋₃alkoxy-C₁₋₃alkyl, wherein said C₃₋₇cycloalkyl, C₁₋₇alkyl, C₂₋₆alkenyl, C₂₋₆alkynyl, C₁₋₇alkoxy, C₃₋₇cycloalkoxy-C₁₋₆alkyl, C₁₋₆alkoxy-C₁₋₆alkyl, C₂₋₆alkenyloxy, C₂₋₆alkenyloxy-C₁₋₆alkyl, C₂₋₆alkynyloxy, C₂₋₆alkynyloxy-C₁₋₆alkyl, C₁₋₆alkylamino, di-C_{C}₁₋₆alkylamino, C₃₋₇heterocycloalkyloxy, C₃₋₇heterocycloalkyl, C₆₋₁₀aryl-C₁₋₃alkoxy, C₆₋₁₀aryl-C₁₋₃alkyl, C₃₋₉heteroaryl-C₁₋₃alkoxy, C₃₋₉heteroaryl-C₁₋₃alkyl, C₃₋₇heterocycloalkyl-C₁₋₃alkoxy, C₃₋₇heterocycloalkyl-C₁₋₃alkyl, C₃₋₇cycloalkyloxy, C₃₋₇cycloalkyl-C₁₋₃alkyl, C₃₋₇cycloalkyl-C₁₋₃alkoxy and C₃₋₇cycloalkyl-C₁₋₃alkoxy-C₁₋₃alkyl are optionally substituted with one or more group selected from phenyl, C₃₋₆cycloalkyl, C₂₋₅heterocycloalkyl, C₃₋₅heteroaryl, —CN, —SR, —OR, —O(CH₂)ₚ—OR, R, —C(═O)—R, —CO₂R, —SO₂R, —SO₂NRR′, halogen, —NO₂, —NRR′, —(CH₂)ₚNRR′, and —C(═O)—NRR′;
each R² is independently selected from halogen, C₁₋₆alkyl, C₃₋₇cycloalkyl, halogenated C₁₋₆alkyl, C₁₋₆alkoxy, and halogenated C₁₋₆alkoxy;
each R³ is independently selected from halogen, C₁₋₆alkyl, C₃₋₇cycloalkyl, halogenated C₁₋₆alkyl, CN, C₁₋₆alkoxy, and halogenated C₁₋₆alkoxy; or two R³ together form a C₁₋₆alkylene, C₁₋₆alkylenoxy, or halogenated C₁₋₆alkylene;
R⁴ is hydrogen, C₁₋₆ alkyl, or C₁₋₆ haloalkyl;
q is 1, 2, 3 or 4;
p is 2, 3 or 4; s is 0, 1, 2, 3, or 4; t is 0, 1, 2, 3, or 4; n is 0,1, 2, 3 or 4; m is 0, 1, 2, 3 or 4;
Y is —CR⁵R⁶—, —O—, or —S—;
X is —CR⁵R⁶—, —NR⁷—, —O—, or —S—;
each R⁵, R⁶ and R⁷ are independently selected from hydrogen, C₁₋₆alkyl, C₂₋₆alkenyl and halogenated C₁₋₆alkyl; and
each R and R′ are independently C₁₋₆alkyl, C₂₋₆alkenyl or halogenated C₁₋₆alkyl, with a proviso that at least one of X and Y is —CR⁵R⁶—.

32. A pharmaceutical composition comprising a compound according to claim 15 and a pharmaceutically acceptable carrier.

33. A method for the therapy of pain in a warm-blooded animal, comprising the step of administering to said animal in need of such therapy a therapeutically effective amount of a compound according to claim 15.

34. A pharmaceutical composition comprising a compound according to claim 16 and a pharmaceutically acceptable carrier.

35. A method for the therapy of pain in a warm-blooded animal, comprising the step of administering to said animal in need of such therapy a therapeutically effective amount of a compound according to claim 16.
